(19) United States
(12) Patent Application Publication
(10) Pub. No.: US 2021/0198644 A1
(43) Pub. Date: Jul. 1, 2021

(54) RECOMBINANT LYSINS

(71) Applicant: The Rockefeller University, New York, NY (US)

(72) Inventors: Vincent FISCHETTI, West Hempstead, NY (US); Assaf RAZ, New York, NY (US); Martin ANDERSSON, Lund (SE)

(21) Appl. No.: 17/057,680

(22) PCT Filed: May 23, 2019
(86) PCT No.: PCT/US2019/033843

§ 371 Date: Nov. 22, 2020

Related U.S. Application Data
(60) Provisional application No. 62/675,210, filed on May 23, 2018.

(51) Int. Cl. C12N 9/36

(52) U.S. Cl. CPC C12N 9/2462; A61K 38/00

(57) ABSTRACT

Provided are methods, compositions and articles of manufacture useful for the prophylactic and therapeutic amelioration and treatment of gram-negative bacteria, including Klebsiella, Enterobacter, and Pseudomonas, and related conditions. The compositions and methods utilize Klebsiellapneumonia, Enterobacter, and Pseudomonas derived bacteriophage lysins, and variants thereof, including truncations thereof.

### CROSS REFERENCE TO RELATED APPLICATION

This application claims priority to U.S. provisional application no. 62/675,210, filed May 23, 2018, the disclosure of which is incorporated herein by reference.

### FIELD

The disclosure relates to bacteriophage lysins that directly kill bacterial cells through hypotonic lysis. These bacteriophage lysins are useful to identify and treat infections caused by pathogenic bacteria.

### BACKGROUND

The global increase of antimicrobial resistance (AMR) is a major public health crisis currently causing about 700,000 annual deaths. Resistance mechanisms against all clinically available antibiotics have been identified, but the emergence of carbapenem-resistant Gram-negative pathogens is particularly worrisome. Generally, carbapenems are saved as a last resort against infections resisting other treatments and there are very few, if any, antibiotics left to use when they fail. As a consequence, the World Health Organization has declared carbapenem-resistant strains of Pseudomonas aeruginosa (CRPA), Acinetobacter baumannii (CRAB) and Enterobacteriaceae (CRE) to be especially urgent threats against global health, and that research on drug development against them is a critical priority.

One of the most clinically important CRE species is Klebsiella pneumoniae, a rod-shaped, encapsulated bacterium naturally present in the environment. It also is a common colonizer of human mucosal tissues. K pneumoniae is known to be a very heterogeneous species with significant genetic variations between strains. This stems from an intrinsic competence to exchange genetic material, which continuously produces strains with new phenotypes. The notable interspecies variation means that different strains utilize different virulence factors during infection and, importantly, cause different types of infections. Today, clinical K. pneumoniae strains can be categorized into two different groups: classical or hyper-virulent K. pneumoniae (cKP or hvKP). Generally, infections by cKP strains are hospital-acquired while hvKP infections are community-acquired.

Classical K. pneumoniae strains are defined by their inability to cause serious infections in immunocompetent individuals. Such strains often colonize human upper respiratory and gastrointestinal tracts but can spread to other tissues and cause severe pneumonias, urine tract infections (UTIs) and bacteremia if the immune system is compromised. For example, cancer patients, chronic alcoholics, diabetics and neonates are all susceptible to cKP infections, which cause ˜12% of all nosocomial pneumonias and 2-6% of UTIs. The pneumonias are often caused by the inhalation of K. pneumoniae colonizing the patient's own oropharyngeal tract or medical ventilator, while nosocomial UTIs can be transmitted by catheters. Bacteremia can either be caused by primary infections of wounds or arise as complications of pneumonias or UTIs. K. pneumoniae is the second most frequent Gram-negative cause of bacteremia, and the mortality rate has been reported to be 27.4-37%. The high mortality can partly be explained by the generally poor health of patients infected by cKP. Another major factor is the widespread resistance to antibiotics, as cKP strains are the primary producers of Klebsiella pneumoniae carbapenemases (KPCs), a group of highly effective β-lactamases. Unlike cKP, hvKPs are capable of infecting otherwise healthy individuals. Such strains are not only able to cause pneumonias, UTIs and bacteremia but also more invasive infections such as meningitis, necrotizing fasciitis, endophthalmitis and abscesses of the kidneys, lungs and liver. A phenotypical difference between cKP and hvKP strains appears to be the structure of their extracellular polysaccharide capsule.

Enterobacter aerogenes and Enterobacter cloacae are a common cause of hospital acquired, multi-drug resistant infection. The genus Enterobacter encompasses organisms that are Gram-negative, rod-shaped, facultative anaerobe, non-spore forming bacteria belonging to the family of Enterobacteriaceae. This genus is genetically related to Klebsiella but separated by their motility (pili are derived from a genomic locus that was acquired from Serratia) as well as the presence of ornithine carboxylase. E. aerogenes is often isolated as clinical specimens from respiratory, urinary, blood, and the GI-tract. ESBL-producing E. aerogenes was associated with several major European outbreaks in the 90s and the 2000s, and antibiotic resistant clones spread rapidly among healthcare facilities. In the 2000s pan-resistant strains of E. aerogenes have emerged, following the acquisition of resistance to last resort antibiotics such as carbapenems and colistin. E. aerogenes represents a substantial burden on the healthcare system. For example, in France it is the fifth most common Enterobacteriaceae, and the seventh most-common Gram-negative rod responsible for hospital-acquired infections.

E. cloacae is an environmental organism commonly found in terrestrial and aquatic environments such as water, sewage, soil, and food. It is also a commensal of the human as well as animals' gut. Like E. aerogenes it is a common source of hospital-acquired infection including bacteremia, endocarditis, septic arthritis, SSTI, lower respiratory tract and urinary tract infections, osteomyelitis, and intra-abdominal infections. It often contaminates medical devices and is common on hospital fomites. It is intrinsically resistant to many beta-lactam antibiotics due to the constitutive production of AmpC beta-lactamase. Plasmid derived expression of AmpC confers resistance to third generation cephalosporins that is transferable among strains. In addition, ESBL producing strains resistant to fourth-generation cephalosporins are a growing concern.

Despite the increasing prevalence of both multidrug resistant and hyper-virulent K. pneumoniae strains, most antibiotics currently in development are targeted against Gram-positive bacteria. The few pipelined drugs against Gram-negatives are mostly modifications of existing drug classes, and resistance mechanisms to these have already been identified. Hence, there is a great need for new classes of drugs targeting Gram-negative bacteria.

In addition to the above-described bacteria, there is also a clear unmet need for the treatment of infections by a variety of Pseudomonas bacteria. For example, multi-drug resistant (MDR) P. aeruginosa colonization and infections in topical and mucosal environments. P. aeruginosa is the second most commonly isolated organisms from patients with ventilator-associated pneumonia (VAP), an infection that has a mortality rate as high as 30% P. aeruginosa topical infections include acute otitis externa (swimmers ear), an infection of the outer ear canal that affects 4 in 1000 people per year, in which 50% of cases are due to P. aeruginosa, and ulcerative keratitis, a bacterial infection causing an inflammatory response of the cornea, often associated with injury or trauma to the cornea or the use of extended-wear soft contact lenses. In burn wound patients, the compromised state of the skin barrier leads to a high risk of infections with P aeruginosa. There is accordingly a clear need for improved compositions and methods for treating these types of bacterial infections. The present disclosure is pertinent to this need.

### SUMMARY

The present disclosure provides pharmaceutical compositions for killing Gram-negative bacteria, including but not necessarily limited to Klebsiella and/or Enterobacter bacteria and/or Pseudomonas bacteria, the pharmaceutical composition comprising at least one isolated lysin polypeptide of Table 1, wherein the isolated lysin polypeptide is an isolated polypeptide comprising one amino acid sequence of Table 1, or variants thereof having at least 80% identity to the least one polypeptide of Table 1, and effective to kill the Gram-negative bacteria. According to another embodiment, the disclosure provides an article of manufacture comprising a vessel containing the lysins and/or their derivatives, and instructions for use of the composition in treatment of a patient exposed to or exhibiting symptoms consistent with exposure to Gram-negative bacteria. In embodiments, the disclosure provides a) identifying an individual suspected of having been exposed to Gram-negative bacteria; and b) administering an effective amount of a pharmaceutical composition as described herein to the individual. According to another embodiment, the disclosure provides a recombinant DNA molecule comprising a DNA sequence or degenerate variant thereof, which encodes a lysin polypeptide from Table 1, or a fragment or variant thereof, DNA sequences that hybridize to any of the foregoing DNA sequences under standard hybridization conditions; and DNA sequences that code on expression for an amino acid sequence encoded by any of the foregoing DNA sequences. According to another embodiment, the disclosure provides a unicellular host transformed with a recombinant DNA molecule that encodes at least one of the lysins or at least one derivative thereof. According to another embodiment, the disclosure provides a method of killing Gram-negative bacteria comprising contacting the bacteria with an effective amount of the one or more of the lysins or derivatives thereof so that some or all of the bacteria are killed. According to another embodiment, the disclosure provides a method for reducing a population of Gram-negative bacteria comprising the step of contacting the bacteria with the one or more of the lysins or derivatives thereof such that at least a portion of the Gram-negative bacteria are killed. According to another embodiment, the disclosure provides a method for treating a Gram-negative bacterial infection in a human or other mammal comprising the step of administering to the human or other mammal having bacterial infection an effective amount one or more of the lysins or derivatives thereof, whereby the number of Gram-negative bacteria in the human or other mammal is reduced and the infection is controlled. According to another embodiment, the disclosure provides a method for treating a human subject exposed to or at risk for exposure to pathogenic Gram-negative bacteria comprising the step of administering to the human subject the composition of claim 1 comprising an amount of the one or more of the lysins or derivatives thereof that is effective to kill the Gram-negative bacteria.

### BRIEF DESCRIPTION OF THE DRAWINGS

FIG. 1 shows the 12 candidate lysins that were screened for abilities to kill Klebsiella species 1_1_55 in HEPES buffer at pH 7.4 (A) and 10% human serum (B). The graphs show the logarithmic changes of 1_1_55 CFU/ml, when incubated with lysins for 1 hr at 37° C. In HEPES, 25 μg/m1 of PlyKp10, PlyKp13 and PlyKp17 decreased the CFU/ml to below the limit of detection (<67 CFU/ml), and all but PlyKp61 and PlyKp68 reduced CFU/ml to some extent (n=2) (A). The standard deviations are shown as error bars. In serum, no lysin appeared to retain its killing activity despite using the maximum possible volume of purified lysins (n=1) (B).

FIG. 2 shows results from incubation of PlyKp17 with three different clinical K. pneumoniae strains, of which one is antibiotic sensitive (PCI 602), one is ESBL-producing (K6) and one is carbapenem-resistant (BIDMC-11). Klebsiella species 1_1_55 was used as a positive control, and all strains were incubated with PlyKp17 for 1 hr at 37° C. in HEPES buffer at pH 7.4. All strains were sensitive to PlyKp17-mediated killing, with a significant reduction observed at 5 μg/ml, and the CFU/ml pushed below the limit of detection (67 CFU/ml, a 5-log reduction) at 25 μg/ml (n=3). Standard deviations are shown as error bars.

FIG. 3 shows the results of incubation of PlyKp17 with M. luteus in 0%, 10%, (A) 25% and 50% (B) human serum and the OD₆₀₀ was measured once every 30 s for 1 hr. PlyKp17 in 10% serum reduced M. luteus OD₆₀₀ quicker than with only serum or lysin alone, indicating an additive effect of lysin and serum components (A). Serum concentrations above 10% reduced the OD₆₀₀ of M. luteus quickly even in the absence of PlyKp17, making it difficult to estimate the activity of the lysin (B).

FIG. 4 shows the results of Klebsiella pneumoniae (PCI 602) incubated with 100 μg/ml PlyKp17-R118 for 1 hr at 37° C. in HEPES supplemented with 0-50% human serum. The CFU/ml was reduced beyond the limit of detection (67 CFU/ml) at 0-1% serum, but no substantial reduction was observed at concentrations above that.

FIG. 5 shows Klebsiella pneumoniae incubated with different concentrations of phage lysin PlyKp104 for 1 h at 37 in 30 mM HEPES pH 7.4. Viable bacteria were quantified by serial dilution and plating. Legend is in μg/ml.

FIG. 6 shows E. coli incubated with different concentrations of phage lysin PlyKp104 for 1 h at 37 in 30 mM HEPES pH 7.4. Viable bacteria were quantified by serial dilution and plating.

FIG. 7 shows E. aerogenes cells were incubated with different concentrations of phage lysin PlyKp104 for 1 h at 37 in 30 mM HEPES pH 7.4. Viable bacteria were quantified by serial dilution and plating.

FIG. 8A shows Acinetobacter baumannii cells incubated with different concentrations of phage lysin PlyKp104 for 1 h at 37 in 30 mM HEPES pH 7.4. Viable bacteria were quantified by serial dilution and plating. Legend is in μg/ml.

FIG. 8B shows Citrobacter freundii cells incubated with different concentrations of phage lysin PlyKp104 for 1 h at 37 in 30 mM HEPES pH 7.4. Viable bacteria were quantified by serial dilution and plating.

FIG. 8C shows Pseudomonas aeruginosa cells incubated with different concentrations of phage lysin PlyKp105 for 1 h at 37 in 30 mM HEPES pH 7.4. Viable bacteria were quantified by serial dilution and plating.

FIG. 9 shows effect of pH on PlyKp104 lysin activity under using sub-MBC lysin concentration to demonstrate difference in activity. Lysins were incubated with Pseudomonas aeruginosa cells at different pH conditions for 1 h at 37° C. Viable bacteria were quantified by serial dilution and plating.

FIG. 10 shows effect of salt on PlyKp104 lysin activity. Lysins were incubated with Pseudomonas aeruginosa cells at different salt concentrations for 1 h at 37° C.

Viable bacteria were quantified by serial dilution and plating.

FIG. 11 shows results of a screen for peptidoglycan hydrolysis activity in Ply307 homologues in Enterobacter aerogenes.

FIG. 12 shows killing assay of Enterobacter aerogenes by purified PlyEa09 lysin. Killing of Enterobacter aerogenes by PlyEa9 following 1 h incubation in 30 mM HEPES buffer at 37° C. Viable bacteria were quantified by serial dilution and plating.

FIG. 13A shows bactericidal activity of lysins against P. aeruginosa PA01. Purified lysins were diluted to various concentrations and incubated with log-phase P. aeruginosa PA01 or Klebsiella sp.HM_44 for 1 h at 37° C. in 30 mM HEPES pH 7.4. CFU/mL values were established by serial dilution and plating. Experiments were conducted in duplicate, error bars represent standard deviation.

FIG. 13B shows bactericidal activity of lysins against Klebsiella sp.HM_44. Purified lysins were diluted to various concentrations and incubated with log-phase Klebsiella sp.HM_44 for 1 h at 37° C. in 30 mM HEPES pH 7.4. CFU/mL values were established by serial dilution and plating. Experiments were conducted in duplicate, error bars represent standard deviation.

FIGS. 13C-D show activity of lysins, PlyPa101, PlyPa103 and PlyKp104 on various bacteria. Various clinical of P. aeruginosa (FIG. 13C), and gram-positive and gram-negative isolates (FIG. 13D) were tested for their sensitivity to the three lysins. All bacteria were incubated with 100 μg/ml of each lysin in 30 mM HEPES buffer pH 7.4 for 1 h at 37° C. Viable bacteria were enumerated by serial dilution and plating. Experiments were done in duplicate, error bars represent standard deviation. FIG. 13C) The Pseudomonas lysins PlyPa103 and Klebsiella lysin PlyKp104, showed the best activity (˜5-log kill) against all the Pseudomonas isolates. D) PlyPa103 and PlyKp104 also had the broadest activity against a variety of gram-negative pathogens including Acinetobacter baumannii, E. coli, Shigella sonnei, Citrobacter freundii, and Proteus mirabilis and no activity against the gram-positive pathogens tested.

FIGS. 13E-F show the effect of pH on the activity of PlyPa101, PlyPa103 and PlyKp104. Log-phase P. aeruginosa PA01 cells were incubated for 1 h at 37° C. with 100 μg/ml lysin in 25 mM of the following buffers: pH 5.0—acetate buffer; pH 6.0—MES buffer; pH 7.0 and 8.0—HEPES buffer; pH 9.0—CHES buffer; pH 10.0—CAPS buffer. Surviving bacterial CFU/ml are presented. Experiments were performed in triplicate, error bars represent standard deviation. FIG. 13E) Results show that PlyKp104 is active against Klebsiella cells in a wide range of pH, from pH 6.0 to 10. FIG. 13F) PlyPa101, PlyPa103 and PlyKp104 was active against Pseudomonas aeruginosa cells from pH 5.0 to 9.0.

FIGS. 13G-H show the effect of NaCl and urea on the activity of PlyPa101, PlyPa103 and PlyKp104. Log-phase P. aeruginosa PA01 cells were incubated with 100 μg/ml PlyPa101, PlyPa103 or PlyKp104 for 1 h at 37° C. in 30 mM HEPES pH 7.4 and various concentrations of NaCl (FIG. 13G) or urea (FIG. 13H). Surviving bacterial CFU/ml are presented; experiments were performed in triplicate. Error bars represent standard deviation. As can be seen salt from 50 mM to 500 mM has little effect on the activity of all three lysins.

FIG. 13I shows that PlyPa101, PlyPa103 and PlyKp104 are active in Survanta. Log-phase P. aeruginosa PA01 cells were incubated for 1 h at 37° C. with 100 μg/ml of PlyPa101, PlyPa103, PlyKp104, or buffer control, in the presence of the indicated concentration of Survanta. Viable bacterial CFU were determined by serial dilution and plating. Experiments were done in triplicate, error bars represent standard deviation. All three lysins are not affected by the presence of the mixed lung surfactant, Survanta at 7.5%.

FIG. 13J shows activity of PlyPa101, PlyPa103 and PlyKp104 in the presence of human serum. P. aeruginosa PA01 cells were incubated for 1 h at 37° C. with 100 μg/ml of the lysins in the presence of the indicated concentration of Serum. Viable bacterial CFU are presented. Experiments were done in triplicate, error bars represent standard deviation. The Klebsiella enzyme PlyKp104, exhibited the best activity in the presence of human serum (up to 6%) with PlyPa103 still active at 3%. PlyPa101 was highly susceptible to the inhibitory activity of serum.

FIG. 14 shows bactericidal activity of lysins against P. aeruginosa PA01. Purified lysins were diluted to various concentrations and incubated with log-phase P. aeruginosa PA01 for 1 h at 37° C. in 30 mM HEPES pH 7.4. CFU/ml values were established by serial dilution and plating. (A) Initial lysins. (B) Additional homologues of PlyPa02. Experiments were conducted in duplicate, error bars represent standard deviation.

FIG. 15 shows activity of the lysins against log-phase and stationary P. aeruginosa. P. aeruginosa were grown overnight (Stat), diluted 1:100 and grown to log-phase (Log). Bacteria were washed and incubated with lysins at the indicated concentrations in 30 mM HEPES buffer pH 7.4 for 1 h at 37° C. Viable bacteria were quantified by serial dilution and plating. Experiments were done in duplicate, error bars represent standard deviation.

FIG. 16 shows activity of lysins against various bacteria. Various isolates of P. aeruginosa (A), Klebsiella and Enterobacter (B), and other Gram-negative and Gram-positive bacteria (C), were incubated with 100 μg/ml lysins in 30 mM HEPES buffer pH 7.4 for 1 h at 37° C. Viable bacteria were enumerated by serial dilution and plating. Experiments were done in duplicate, error bars represent standard deviation. For each bar set on the X-axis, the order of the five bars in each set is: PlyPa01, PlyPa03, PlyPa91, PlyPa96, and Control. The control is represented by the unshaded bar in each set.

FIG. 17 shows a time kill curve—Log-phase P. aeruginosa PA01 cells were incubated for varying lengths of time at 37° C. with 100 μg/ml lysin in 30 mM HEPES buffer. Surviving bacteria were enumerated by serial dilution and plating, experiments were done in triplicates; error bars represent standard deviation.

FIG. 18 shows the effect of pH on the activity of PlyPa03 and PlyPa91. Log-phase P. aeruginosa PA01 cells were incubated for 1 h at 37° C. with 100 μg/ml lysin in 25 mM of the following buffers: pH 5.0—acetate buffer; pH 6.0—MES buffer; pH 7.0and 8.0—HEPES buffer; pH 9.0—CHES buffer; pH 10.0—CAPS buffer. Surviving bacterial CFU/ml are presented. Experiments were performed in triplicate, error bars represent standard deviation.

FIG. 19 shows the effect of NaCl and urea on the activity of PlyPa03 and PlyPa91. Log-phase P. aeruginosa PA01 cells were incubated with 100 μg/ml PlyPa03, or PlyPa91 for 1 h at 37° C. in 30 mM HEPES pH 7.4 and various concentrations of NaCl (A) or urea (B). Surviving bacterial CFU/ml are presented; experiments were performed in triplicate. Error bars represent standard deviation.

FIG. 20 shows the elimination of P. aeruginosa biofilm by PlyPa03 and PlyPa91. P. aeruginosa PA01 biofilm was established using the MBEC Biofilm Inoculator 96-well plate system. Biofilms were grown for 24 h on the 96-peg lid, washed twice, and treated with different concentrations of PlyPa03, PlyPa91, of buffer control for 2 h at 37° C. The pegs were washed, and surviving bacteria were recovered by sonication in 200 μl/well PBS. Quantification of surviving bacteria was done by serial dilution and plating. Experiments were done in triplicate, error bars represent standard deviation.

FIG. 21 shows the activity of PlyPa03 and PlyPa91 in the presence of human serum and Survanta. Log-phase P. aeruginosa PA01 cells were incubated for 1 h at 37° C. with 100 μg/ml of PlyPa03, PlyPa91, or buffer control, in the presence of the indicated concentration of Serum (A) or Survanta (B). Viable bacterial CFU were determined by serial dilution and plating. Experiments were done in triplicate, error bars represent standard deviation.

FIG. 22. PlyPa03 and PlyPa91 are not cytotoxic to human cells. (A) Human red blood cells from healthy donors were suspended in PBS and incubated with PlyPa03 or PlyPa91 at concentrations ranging from 1 to 200 μg/ml, for 4 h at 37° C. PBS was used as negative control and 1% triton X-100 was used as positive control. Hemoglobin release was evaluated by measuring absorbance at 405 nm following removal of intact cells. (B) HL-60 neutrophils were incubated with HBSS containing various concentrations of PlyPa03 or PlyPa91, in a 96-well plate for 4 h at 37° C. 5% CO2. Tetrazolium substrate was added for 4 hours, and stop solution was added overnight. Absorbance was measured at OD570 nm to evaluate conversion of tetrazolium into a formazan by live cells. 1% Triton X-100 served as positive control and PBS as negative control. Assays were carried out in triplicates, error bars represent standard deviation.

FIG. 23 shows PlyPa03 protects mice in a skin infection model. A skin area on the backs of CD1 female mice was shaven and tape-stripped, and then infected with 10 pi log-phase P. aeruginosa at 5×10⁶ CFU/ml. After 20 hours, the mice were treated with PlyPa03, PlyPa91, or buffer control, and were euthanized 3 hours later. The infected skin was immediately excised and homogenized in PBS, and the resulting liquid was serially diluted and plated for CFU quantification. Geometric mean of the values is presented. Panels A and B represent two separate experiments.

FIG. 24 shows PlyPa91 protects mice in a lung infection model. Lungs of female C57BL/6 mice were infected by intranasal application of 2×50 μl of 10⁸ CFU/ml log-phase P. aeruginosa PA01 by intranasal instillation. At three and six hours post infection mice were treated with 50 μl of 1.8 mg/ml PlyPa91 or PBS by two intranasal instillations (nasal delivery) or by one intranasal and one intratracheal instillation (nasal & lung delivery);

PBS controls from the two treatment regiments were combined in a single group. 10-day survival was analyzed using Kaplan-Meier survival curves with standard errors, 95% confidence intervals, and significance levels (log rank/Mantel-Cox test). Results presented were combined from three separate experiments.

FIG. 25 shows the evaluation of lysin peptidoglycan hydrolase activity using the plate overlay method. E. coli strains containing lysin genes in pAR553 were grown on a plate containing 0.2% arabinose to induce lysin expression. Cells were permeabilized with chloroform vapor and overlayed with soft agar containing autoclaved P. aeruginosa cells. Enzymatic activity was evaluated by the appearance of clearing zones.

FIG. 26 shows the evaluation of lysin peptidoglycan hydrolase activity in crude lysate. Induced crude lysates of E. coli strains harboring the lysin genes in pAR553 were spotted in different amounts on a plate containing soft agar with autoclaved P. aeruginosa. Enzymatic activity was evaluated by the appearance of clearing zones.

FIG. 27 shows the purification of PlyPa02. A PlyPa02 fused to a 3C-cleavable hexahistidine tag was purified from an induced E. coli lysate by a single step metal affinity chromatography: L—Induced lysate; fractions 1-5—load; fractions 6-15—wash steps; fractions 16-18—collected elution; fractions 23-29—column regeneration. Coomassie stain of a 15% SDS-PAGE containing select fractions.

FIG. 28 shows the cleavage of PlyPa02 with various doses of 3C protease. Reaction mixtures with a total volume of 20 μl were prepared by combining 10 μg of PlyPa02, 2 μl of 4-fold serially diluted 3C protease and the following buffer composition: 150 mM NaCl; 50 mM tris; 10 mM EDTA; and 1 mM DTT, pH 7.6. Reactions were incubated at 4° C. for 16 h, samples were loaded on 15% SDS-PAGE, and the gel stained with Coomassie blue.

FIG. 29 shows the activity of lysins against P. aeruginosa strains at 250 μg/ml. P. aeruginosa strains PA01, AR463, and AR463 were incubated with 250 μg/ml of the lysins in 30 mM HEPES buffer pH 7.4 for 1 h at 37° C. Viable bacteria were enumerated by serial dilution and plating. Experiments were done in duplicate, error bars represent standard deviation.

FIG. 30 shows the effect of pH on the activity of (A) PlyPa03 and (B) PlyPa91. Log-phase P. aeruginosa PA01 cells were incubated for 1 h at 37° C. with various lysin concentrations in 25 mM of the following buffers: pH 6.0—MES buffer; pH 7.0 and 8.0—HEPES buffer; pH 9.0—CHES buffer. Surviving bacterial CFU/ml are presented; experiments were performed in triplicates. Error bars represent standard deviation.

FIG. 31 shows the effect of EDTA on lysin activity. Log-phase P. aeruginosa PA01 cells were incubated for 1 h at 37° C. with serially diluted PlyPa03 or PlyPa91 in the presence or absence of 0.5 mM EDTA. Viable bacterial CFU are presented. Experiments were done in triplicates.

### DETAILED DESCRIPTION

Where a value of ranges is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges which may independently be included in the smaller ranges is also encompassed within the disclosure, subject to any specifically excluded limit in the stated range. Where the stated range includes one or both of the limits, ranges excluding either both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, exemplary methods and materials are now described. All publications mentioned herein are incorporated herein by reference to disclose and describe the methods and/or materials in connection with which the publications are cited.

As used herein and in the appended claims, the singular forms “a”, “and” and “the” include plural references unless the context clearly dictates otherwise. All technical and scientific terms used herein have the same meaning.

Antibiotic resistant infections are becoming increasingly problematic, and some pathogens are now resistant to all available drugs. In particular, multidrug-resistant Gram-negatives such as carbapenem-resistant Klebsiella pneumoniae can cause high-mortality infections due to the lack of effective treatments. The disclosure provides bacteriophage lysins effective against multidrug-resistant K. pneumonia, as well as other Gram-negative bacteria that are described further below.

The disclosure takes advantage of bacteriophage lysins, as described further below. In this regard, it is known in the art that bacteriophages infect their host bacteria to produce more virus particles. At the end of the reproductive cycle they are faced with a problem; how to release the phage progeny trapped within the bacterium. They solve this problem by producing an enzyme termed “lysin” that degrades the cell wall of the infected bacteria to release the phage progeny. The lytic system contains a holin and at least one peptidoglycan hydrolase, or lysin, capable of degrading the bacterial cell wall. Lysins can be endo-β-N-acetylglucosaminidases or N-acetyl-muramidases (lysozymes), which act on the sugar moiety, endopeptidases which act on the peptide backbone or cross bridge, or more commonly, an N-acetylmuramoyl-L-alanine amidase (or amidase), which hydrolyzes the amide bond connecting the sugar and peptide moieties. Typically, the holin is expressed in the late stages of phage infection forming a pore in the inner cell membrane, thus granting the lysin access to its substrate, the peptidoglycan, eventually resulting in lysis and the release of progeny phage. Significantly, exogenously added lysin can lyse the cell wall of healthy, uninfected cells, producing a phenomenon known as “lysis from without”. This strategy has proven effective for several different Gram-positive bacteria. However, prior to the present disclosure, gram-negative bacteria have generally so far proven highly resistant to the addition of exogenously added lysins due to their protective outer membrane, unless the lysin is added together with membrane destabilizing factors. However, a small fraction of lysins display a low innate ability to kill Gram-negative bacteria, an ability that is highly improved in the presence of membrane destabilizing factors. This innate ability has been thought to be due to the presence of highly charged N- or C-terminal peptides fused to the catalytic domain of the lysin, thus helping the lysins to penetrate the outer membrane and reach their peptidoglycan substrate. Artilysins, engineered lysins with added peptides for improved antibacterial activity, have been reported. In contrast, the present disclosure describes native antibacterial proteins present in Gram-negative phages, and provides derivatives of such native proteins.

### GLOSSARY

The terms “Klebsiella pneumoniae lysin(s)”, as used throughout the present application and claims refer to proteinaceous material including single or multiple proteins, and extends to those proteins having the amino acid sequence data described herein and presented in Table 1, and the profile of activities set forth herein and in the Claims. Accordingly, proteins displaying substantially equivalent or altered activity are likewise contemplated. These modifications may be deliberate, for example, such as modifications obtained through site-directed mutagenesis, or may be accidental, such as those obtained through mutations in hosts that are producers of the complex or its named subunits. Also, the term “Klebsiella lysins” is intended to include within its scope proteins specifically recited herein as well as all substantially homologous analogs, fragments or truncations, and allelic variations. The terms Klebsiella pneumoniae lysin and Klebsiella lysin are adapted with the same meaning, except as modified to refer to the particular organism for which the bacteriophage are specific, and from which the particular lysin is obtained and/or are derived.

### Polypeptides and Lytic Enzymes

A “lytic enzyme” includes any bacterial cell wall lytic enzyme that kills one or more bacteria under suitable conditions and during a relevant time period. Examples of lytic enzymes include, without limitation, various amidase, glucosaminidase, muramidase, endopeptidase cell wall lytic enzymes.

A “Klebsiella enzyme” includes a lytic enzyme that is capable of killing at least one or more Klebsiella bacteria under suitable conditions and during a relevant time period. Other types of bacteria may also be killed by a Klebsiella enzyme.

A “Pseudomonas enzyme” includes a lytic enzyme that is capable of killing at least one or more Pseudomonas bacteria under suitable conditions and during a relevant time period. Other types of bacteria may also be killed by a Pseudomonas enzyme.

A “bacteriophage lytic enzyme” refers to a lytic enzyme extracted, isolated from a bacteriophage or a synthesized lytic enzyme with a similar protein structure that maintains a lytic enzyme functionality.

A lytic enzyme is capable of specifically cleaving bonds that are present in the peptidoglycan of bacterial cells. Since bacteria are under high pressure any cleavage of the bonds in the peptidoglycan will disrupt the bacterial cell wall. It is also currently postulated that the bacterial cell wall peptidoglycan is highly conserved among most bacteria, and cleavage of only a few bonds may disrupt the bacterial cell wall. The bacteriophage lytic enzyme may be an amidase, although other types of enzymes are possible. Examples of lytic enzymes that cleave these bonds are muramidases, glucosaminidases, endopeptidases, or N-acetyl-muramoyl-L-alanine amidases (or amidase for short). Fischetti et al (1974) reported that the C1 streptococcal phage lysin enzyme was an amidase. Garcia et al (1987, 1990) reported that the Cpl lysin from a S. pneumoniae from a Cp-1 phage was a lysozyme. Caldentey and Bamford (1992) reported that a lytic enzyme from the phi 6 Pseudomonas phage was an endopeptidase, splitting the peptide bridge formed by melo-diaminopimilic acid and D-alanine. The E. coli T1 and T6 phage lytic enzymes are amidases as is the lytic enzyme from Listeria phage (ply) (Loessner et al, 1996). There are also many other lytic enzymes known in the art that are capable of cleaving a bacterial cell wall.

A “lytic enzyme genetically coded for by a bacteriophage” includes a polypeptide capable of killing a host bacteria, for instance by having at least some cell wall lytic activity against the host bacteria. The polypeptide may have a sequence that encompasses native sequence lytic enzyme and variants thereof. The polypeptide may be isolated from a variety of sources, such as from a bacteriophage (“phage”), or prepared by recombinant or synthetic methods, such as those described by Garcia et al and also as provided herein. Generally speaking, a lytic enzyme may be between 20,000 and 45,000 daltons in molecular weight and comprise a single polypeptide chain; however, this can vary depending on the enzyme chain.

A “native sequence phage associated lytic enzyme” includes a polypeptide having the same amino acid sequence as an enzyme derived from a bacteria. Such native sequence enzyme can be isolated or can be produced by recombinant or synthetic means.

The term “native sequence enzyme” encompasses naturally occurring forms (for example, alternatively spliced or altered forms) and naturally-occurring variants of the enzyme. In one embodiment of the disclosure, the native sequence enzyme is a mature or full-length polypeptide that is genetically coded for by a gene from a bacteriophage specific for Klebsiella pneumonia. In another embodiment, the native sequence enzyme is a mature or full-length polypeptide that is genetically coded for by a gene from a bacteriophage specific for Pseudomonas aeruginosa.

“A variant sequence lytic enzyme” includes a lytic enzyme characterized by a polypeptide sequence that is different from that of a lytic enzyme, but retains functional activity. The lytic enzyme can, in some embodiments, be genetically coded for by a bacteriophage specific for Klebsiella pneumoniae having a particular amino acid sequence identity with the lytic enzyme sequence(s) hereof, as in Table 1, and in other tables of this disclosure. For example, in some embodiments, a functionally active lytic enzyme can kill Klebsiella pneumoniae bacteria, and other susceptible bacteria as provided herein, including as shown in Table 1 and other tables herein, by disrupting the cellular wall of the bacteria. An active lytic enzyme may have a 60, 65, 70, 75, 80, 85, 90, 95, 97, 98, 99 or 99.5% amino acid sequence identity with the lytic enzyme sequence(s) hereof, as provided in Table 1, and in other tables and the description of this disclosure that include amino acid sequences, or other amino acid identifying information. Such phage associated lytic enzyme variants include, for instance, lytic enzyme polypeptides wherein one or more amino acid residues are added, or deleted at the N or C terminus of the sequence of the lytic enzyme sequence(s) hereof, as provided in Table 1, and other tables as will be apparent from this disclosure. In a particular aspect, a phage associated lytic enzyme will have at least about 80% or 85% amino acid sequence identity with native phage associated lytic enzyme sequences, particularly at least about 90% (e.g. 90%) amino acid sequence identity. Most particularly a phage associated lytic enzyme variant will have at least about 95% (e.g. 95%) amino acid sequence identity with the native phage associated the lytic enzyme sequence(s) hereof, as provided in Table 1.

“Percent amino acid sequence identity” with respect to the phage associated lytic enzyme sequences identified is defined herein as the percentage of amino acid residues in amino acid candidate sequence that are identical with the amino acid residues in the phage associated lytic enzyme sequence, after aligning the sequences in the same reading frame and introducing gaps, if necessary, to achieve the maximum percent sequence identity, and not considering any conservative substitutions as part of the sequence identity.

“Percent nucleic acid sequence identity” with respect to the phage associated lytic enzyme sequences identified herein is defined as the percentage of nucleotides in a sequence that are identical with the nucleotides in the phage associated lytic enzyme sequence, after aligning the sequences and introducing gaps, if necessary, to achieve the maximum percent sequence identity.

“Polypeptide” includes a polymer molecule comprised of multiple amino acids joined in a linear manner. A polypeptide can, in some embodiments, correspond to molecules encoded by a polynucleotide sequence which is naturally occurring. The polypeptide may include conservative substitutions where the naturally occurring amino acid is replaced by one having similar properties, where such conservative substitutions do not alter the function of the polypeptide (see, for example, Lewin “Genes V” Oxford University Press Chapter 1, pp. 9-13 1994).

The term “altered lytic enzyme” includes shuffled and/or chimeric lytic enzymes, or enzymes that have been made recombinantly to include one or more amino acids, or fewer amino acids, such that the altered lytic enzyme is different from the lytic enzyme as produced by unmodified bacteria as a component of a phage.

A lytic enzyme or polypeptide of the disclosure may be produced in the bacterial organism after being infected with a particular bacteriophage. This naturally produced lysin is used to release the phage progeny by lysing the phage-infected bacterial cell. The lytic enzyme(s) or polypeptide(s) may be truncated, chimeric, shuffled or “natural,” and may be in combination. An “altered” lytic enzyme can be produced in a number of ways. In one embodiment, a gene for the altered lytic enzyme from the phage genome is put into a transfer or movable vector, such as a plasmid, and the plasmid is cloned into an expression vector or expression system. The expression vector for producing a lysin polypeptide or enzyme of the disclosure may be suitable for E. coli, Bacillus, or a number of other suitable bacteria. The vector system may also be a cell free expression system. All of these methods of expressing a gene or set of genes are known in the art.

A “chimeric protein” or “fusion protein” comprises all or a biologically active part of a polypeptide of the disclosure operably linked to a heterologous polypeptide.

A “heterologous” region of a DNA construct or protein or peptide construct is an identifiable segment of DNA within a larger DNA molecule or peptide or protein within a larger molecule that is not found in association with the larger molecule in nature.

The term “operably linked” means that the polypeptide of the disclosure and the heterologous polypeptide are fused in-frame. The heterologous polypeptide can be fused to the N-terminus or C-terminus of the polypeptide of the disclosure. Chimeric proteins are produced enzymatically by chemical synthesis, or by recombinant DNA technology. One example of a useful fusion protein is a GST fusion protein in which the polypeptide of the disclosure is fused to the C-terminus of a GST sequence. Such a protein can facilitate the purification of a recombinant polypeptide of the disclosure.

In another embodiment, the chimeric protein or peptide contains a heterologous signal sequence at its N-terminus. For example, the native signal sequence of a polypeptide of the disclosure can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992, incorporated herein by reference). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

The fusion protein can combine a lysin polypeptide with a protein or polypeptide of having a different capability, or providing an additional capability or added character to the lysin polypeptide. The fusion protein may be an immunoglobulin fusion protein in which all or part of a polypeptide of the disclosure is fused to sequences derived from a member of the immunoglobulin protein family. The fusion gene can be synthesized by conventional techniques, including automated DNA synthesizers.

As used herein, shuffled proteins or peptides, gene products, or peptides for more than one related phage protein or protein peptide fragments have been randomly cleaved and reassembled into a more active or specific protein. Shuffled oligonucleotides, peptides or peptide fragment molecules are selected or screened to identify a molecule having a desired functional property.

Modified or altered form of the protein or peptides and peptide fragments, as disclosed herein, includes protein or peptides and peptide fragments that are chemically synthesized or prepared by recombinant DNA techniques, or both. Certain preparations of the proteins described herein have less than about 30%, 20%, 10%, 5% (by dry weight) of chemical precursors or compounds other than the polypeptide of interest.

A signal sequence of a polypeptide can facilitate transmembrane movement of the protein and peptides and peptide fragments of the disclosure to and from mucous membranes, as well as by facilitating secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the disclosure can pertain to the described polypeptides having a signal sequence, as well as to the signal sequence itself and to the polypeptide in the absence of the signal sequence (i.e., the cleavage products). A nucleic acid sequence encoding a signal sequence of the disclosure can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art-recognized methods. Alternatively, the signal sequence can be linked to a protein of interest using a sequence which facilitates purification, such as with a GST domain.

The present disclosure also pertains to other variants of the polypeptides described herein. Variants can be generated by mutagenesis, i.e., discrete point mutation or truncation. Variants can retain substantially the same, or a subset, of the biological activities of the naturally occurring form of the protein.

Treatment of a subject with a variant having a subset of the biological activities of the naturally occurring form of the protein can have fewer side effects in a subject relative to treatment with the naturally occurring form of the protein.

The smallest polypeptide (and associated nucleic acid that encodes the polypeptide) that can be expected to function in methods of this disclosure, may be 8, 9, 10, 11, 12, 13, 15, 20, 25, 30, 35, 40, 45, 50, 55, 60, 65, 75, 85, or 100 amino acids long. Smaller sequences as short as 8, 9, 10, 11, 12 or 15 amino acids long are also include. Thus, the smallest portion of the protein(s) or lysin polypeptides provided herein, including as in Table 1, and other tables of this disclosure, includes polypeptides as small as 5, 6, 7, 8, 9, 10, 12, 14 or 16 amino acids long.

Biologically active portions of a protein or peptide fragment of the embodiments, as described herein, include polypeptides comprising amino acid sequences sufficiently identical to or derived from the amino acid sequence of the phage protein of the disclosure, which include fewer amino acids than the full length protein of the phage protein and exhibit at least one activity of the corresponding full-length protein. Typically, biologically active portions comprise a domain or motif with at least one activity of the corresponding protein. A biologically active portion of a protein or protein fragment of the disclosure can be a polypeptide which is, for example, 10, 25, 50, 100 less or more amino acids in length. Moreover, other biologically active portions, in which other regions of the protein are deleted, or added can be prepared by recombinant techniques and evaluated for one or more of the functional activities of the native form of a polypeptide of the embodiments.

Most lysins have domains which function differently to achieve the final lytic event. Charged domains (negatively or positively) may be found at one or both ends of a central catalytic domain that is responsible for cleaving a bond in the peptidoglycan. Each domain may also be separated from the whole molecule to be used independently to disrupt the bacterial cell wall. Each domain may also be modified by other catalytic or charged domains to improve their activity. Each domain may be fused at either end to antimicrobial peptides of mammalian origin to improve the activity of either molecule for bacterial killing. Homologous proteins and nucleic acids can be prepared that share functionality with such small proteins and/or nucleic acids (or protein and/or nucleic acid regions of larger molecules) as will be appreciated by a skilled artisan. Such small molecules and short regions of larger molecules that may be homologous specifically are intended as embodiments. In embodiments, the homology of such valuable regions is at least 50%, 65%, 75%, 80%, 85%, and in certain embodiments, at least 90%, 95%, 97%, 98%, or at least 99% compared to the lysin polypeptides provided herein, including as set out in Table 1, and all amino acid sequences otherwise described herein. These percent homology values do not include alterations due to conservative amino acid substitutions.

Amino acid sequences of the present disclosure should be considered to include sequences containing conservative changes which do not significantly alter the activity or binding characteristics of the resulting protein.

Thus, one of skill in the art, based on a review of the sequence of the lysin polypeptides provided herein and on their knowledge and the public information available for other lysin polypeptides, can make amino acid changes or substitutions in the lysin polypeptide sequence. Amino acid changes can be made to replace or substitute one or more, one or a few, one or several, one to five, one to ten, or such other number of amino acids in the sequence of the lysin(s) provided herein to generate mutants or variants thereof. Such mutants or variants thereof may be predicted for function or tested for function or capability for killing bacteria, and/or for having comparable activity to the lysin(s) provided herein.

The term “specific” may be used to refer to the situation in which one member of a specific binding pair will not show significant binding to molecules other than its specific binding partner(s). The term “comprise” generally used in the sense of include, that is to say permitting the presence of one or more features or components.

The term “consisting essentially of” refers to a product, particularly a peptide sequence, of a defined number of residues which is not covalently attached to a larger product. In the case of the polypeptides of this disclosure, those of skill in the art will appreciate that minor modifications to the N- or C-terminal of the peptide may however be contemplated, such as the chemical modification of the terminal to add a protecting group or the like, e.g. the amidation of the C-terminus.

The term “Isolated” refers to the state in which the lysin polypeptide(s) of the disclosure, or nucleic acid encoding such polypeptides will be, in accordance with the present disclosure. Polypeptides and nucleic acid will be free or substantially free of material with which they are naturally associated such as other polypeptides or nucleic acids with which they are found in their natural environment, or the environment in which they are prepared (e.g. cell culture) when such preparation is by recombinant DNA technology practiced in vitro or in vivo.

### Nucleic Acids

Nucleic acids capable of encoding all of the polypeptide(s) of the disclosure are provided herein and constitute an aspect of the disclosure.

A wide variety of unicellular host cells are useful in expressing the DNA sequences of this disclosure. These hosts may include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

### Compositions

Therapeutic or pharmaceutical compositions comprising the lytic enzyme(s)/polypeptide(s) of the disclosure are provided in accordance with the disclosure, as well as related methods of use and methods of manufacture. Therapeutic or pharmaceutical compositions may comprise one or more lytic polypeptide(s), and optionally include natural, truncated, chimeric or shuffled lytic enzymes, optionally combined with other components such as a carrier, vehicle, polypeptide, polynucleotide, holin protein(s), one or more antibiotics or suitable excipients, carriers or vehicles. The disclosure provides therapeutic compositions or pharmaceutical compositions of the lysins of the disclosure, including those described in Table 1 and throughout this disclosure, for use in the killing, alleviation, decolonization, prophylaxis or treatment of Gram-positive or gram-negative bacteria, including bacterial infections or related conditions. The disclosure provides therapeutic compositions or pharmaceutical compositions of the lysins of the disclosure, including those of Table 1 and throughout this specification, for use in treating, reducing or controlling contamination and/or infections by Gram-positive or Gram-negative bacteria, including in contamination or infection. Compositions are thereby contemplated and provided for therapeutic applications and local or systemic administration. Compositions comprising the polypeptides described herein, including truncations or variants thereof, are provided herein for use in the killing, alleviation, decolonization, prophylaxis or treatment of gram-positive or Gram-negative bacteria, including bacterial infections or related conditions, particularly Klebsiella pneumonia, Pseudomonas aeruginosa and Staphylococcus aureus. The enzyme(s) or polypeptide(s) included in the therapeutic compositions may be one or more or any combination of unaltered phage associated lytic enzyme(s), truncated lytic polypeptides, variant lytic polypeptide(s), and chimeric and/or shuffled lytic enzymes. Additionally, different lytic polypeptide(s) genetically coded for by different phage for treatment of the same bacteria may be used. These lytic enzymes may also be any combination of unaltered lytic enzymes or polypeptides, truncated lytic polypeptide(s), variant lytic polypeptide(s), and chimeric and shuffled lytic enzymes or domains thereof. The lytic enzyme(s)/polypeptide(s) in a therapeutic or pharmaceutical composition for gram-negative bacteria may be used alone or in combination with antibiotics or, if there are other invasive bacterial organisms to be treated, in combination with other phage associated lytic enzymes specific for other bacteria being targeted. Any polypeptide described herein made used in connection with a holin protein.

The pharmaceutical composition can contain a complementary agent, including one or more antimicrobial agent and/or one or more conventional antibiotics. In order to accelerate treatment of the infection, the therapeutic agent may further include at least one complementary agent which can also potentiate the bactericidal activity of the lytic enzyme.

Also provided are compositions containing nucleic acid molecules that, either alone or in combination with other nucleic acid molecules, are capable of expressing an effective amount of a lytic polypeptide(s) or a peptide fragment of a lytic polypeptide(s) in vivo. Cell cultures containing these nucleic acid molecules, polynucleotides, and vectors carrying and expressing these molecules in vitro or in vivo, are also provided.

Therapeutic or pharmaceutical compositions may comprise lytic polypeptide(s) combined with a variety of carriers to treat the illnesses caused by the susceptible gram-positive bacteria. The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; glycine; amino acids such as glutamic acid, aspartic acid, histidine, or arginine; monosaccharides, disaccharides, and other carbohydrates including cellulose or its derivatives, glucose, mannose, trehalose, or dextrins; chelating agents such as EDTA; sugar alcohols such as mannitol or sorbitol; counter-ions such as sodium; non-ionic surfactants such as polysorbates, poloxamers, or polyethylene glycol (PEG); and/or neutral salts, e.g., NaCl, KCl, MgCl₂, CaCl₂, and others. Glycerin or glycerol (1,2,3-propanetriol) is commercially available for pharmaceutical use. It may be diluted in sterile water for injection, or sodium chloride injection, or other pharmaceutically acceptable aqueous injection fluid, and used in concentrations of 0.1 to 100% (v/v), or 1.0 to 50%, or about 20%. DMSO is an aprotic solvent with a remarkable ability to enhance penetration of many locally applied drugs. DMSO may be diluted in sterile water for injection, or sodium chloride injection, or other pharmaceutically acceptable aqueous injection fluid, and used in concentrations of 0.1 to 100% (v/v). The carrier vehicle may also include Ringer's solution, a buffered solution, and dextrose solution, particularly when an intravenous solution is prepared.

Any of the carriers for the lytic polypeptide(s) may be manufactured by conventional means. However, in certain embodiments, any mouthwash or similar type products do not contain alcohol to prevent denaturing of the polypeptide/enzyme. Similarly, when the lytic polypeptide(s) is being placed in a cough drop, gum, candy or lozenge during the manufacturing process, such placement should be made prior to the hardening of the lozenge or candy but after the cough drop or candy has cooled somewhat, to avoid heat denaturation of the enzyme.

A lytic polypeptide(s) may be added to these substances in a liquid form or in a lyophilized state, whereupon it will be solubilized when it meets body fluids such as saliva. The polypeptide(s)/enzyme may also be in a micelle or liposome.

The effective dosage rates or amounts of an altered or unaltered lytic enzyme/polypeptide(s) to treat the infection will depend in part on whether the lytic enzyme/polypeptide(s) will be used therapeutically or prophylactically, the duration of exposure of the recipient to the infectious bacteria, the size and weight of the individual, etc. The duration for use of the composition containing the enzyme/polypeptide(s) also depends on whether the use is for prophylactic purposes, wherein the use may be hourly, daily or weekly, for a short time period, or whether the use will be for therapeutic purposes wherein a more intensive regimen of the use of the composition may be needed, such that usage may last for hours, days or weeks, and/or on a daily basis, or at timed intervals during the day. Any dosage form employed should provide for a minimum number of units or micrograms (μg) for a minimum amount of time. The concentration of the active units or ug of enzyme believed to provide for an effective amount or dosage of enzyme may be in the range of about 100 units/ml (10 μg/m1) to about 500,000 units/ml (100 ug/ml) of fluid in the wet or damp environment of the nasal and oral passages, and possibly in the range of about 100 units/ml (10 ug/ml) to about 50,000 units/ml (50 ug/ml). More specifically, time exposure to the active enzyme/polypeptide(s) units may influence the desired concentration of active enzyme units per ml. Carriers that are classified as “long” or “slow” release carriers (such as, for example, certain nasal sprays or lozenges) could possess or provide a lower concentration of active (enzyme) units per ml, but over a longer period of time, whereas a “short” or “fast” release carrier (such as, for example, a gargle) could possess or provide a high concentration of active (enzyme) units per ml, but over a shorter period of time. The amount of active units per ml and the duration of time of exposure depend on the nature of infection, whether treatment is to be prophylactic or therapeutic, and other variables. There are situations where it may be necessary to have a much higher unit/ml dosage or a lower unit/ml dosage.

The lytic enzyme/polypeptide(s) can be in an environment having a pH which allows for activity of the lytic enzyme/polypeptide(s). For example if a human individual has been exposed to another human with a bacterial upper respiratory disorder, the lytic enzyme/polypeptide(s) will reside in the mucosal lining and prevent any colonization of the infecting bacteria. Prior to, or at the time the altered lytic enzyme is put in the carrier system or oral delivery mode, in embodiments, the enzyme may be in a stabilizing buffer environment for maintaining a pH range between about 4.0 and about 9.0, or between about 5.5 and about 7.5.

A stabilizing buffer may allow for the optimum activity of the lysin enzyme/polypeptide(s). The buffer may contain a reducing reagent, such as dithiothreitol. The stabilizing buffer may also be or include a metal chelating reagent, such as ethylenediaminetetracetic acid disodium salt, or it may also contain a phosphate or citrate-phosphate buffer, or any other buffer. The DNA coding of these phages and other phages may be altered to allow a recombinant enzyme to attack one cell wall at more than two locations, to allow the recombinant enzyme to cleave the cell wall of more than one species of bacteria, to allow the recombinant enzyme to attack other bacteria, or any combinations thereof. The type and number of alterations to a recombinant bacteriophage produced enzyme are incalculable.

A mild surfactant can be included in a therapeutic or pharmaceutical composition in an amount effective to potentiate the therapeutic effect of the lytic enzyme/polypeptide(s) may be used in a composition. Suitable mild surfactants include, inter alia, esters of polyoxyethylene sorbitan and fatty acids (Tween series), octylphenoxy polyethoxy ethanol (Triton-X series), n-Octyl-.beta.-D-glucopyranoside, n-Octyl-.beta.-D-thioglucopyranoside, n-Decyl-.beta.-D-glucopyranoside, n-Dodecyl-.beta.-D-glucopyranoside, and biologically occurring surfactants, e.g., fatty acids, glycerides, monoglycerides, deoxycholate and esters of deoxycholate.

Preservatives may also be used in this disclosure and may comprise about 0.05% to 0.5% by weight of the total composition. The use of preservatives assures that if the product is microbially contaminated, the formulation will prevent or diminish microorganism growth. Some preservatives useful in this disclosure include methylparaben, propylparaben, butylparaben, chloroxylenol, sodium benzoate, DMDM Hydantoin, 3-Iodo-2-Propylbutyl carbamate, potassium sorbate, chlorhexidine digluconate, or a combination thereof

Pharmaceuticals for use in embodiments of the disclosure include also include anti-inflammatory agents, antiviral agents, local anesthetic agents, corticosteroids, destructive therapy agents, antifungals, and antiandrogens. In embodiments, active pharmaceuticals that may be used include antimicrobial agents, especially those having anti-inflammatory properties such as dapsone, erythromycin, minocycline, tetracycline, clindamycin, and other antimicrobials. Weight percentages for the antimicrobials are generally 0.5% to 10%.

Local anesthetics include tetracaine, tetracaine hydrochloride, lidocaine, lidocaine hydrochloride, dyclonine, dyclonine hydrochloride, dimethisoquin hydrochloride, dibucaine, dibucaine hydrochloride, butambenpicrate, and pramoxine hydrochloride. A representative concentration for local anesthetics is about 0.025% to 5% by weight of the total composition. Anesthetics such as benzocaine may also be used at, for example, a concentration of about 2% to 25% by weight.

Corticosteroids that may be used include betamethasone dipropionate, fluocinolone actinide, betamethasone valerate, triamcinolone actinide, clobetasol propionate, desoximetasone, diflorasone diacetate, amcinonide, flurandrenolide, hydrocortisone valerate, hydrocortisone butyrate, and desonide are recommended at concentrations of about 0.01% to 1.0% by weight. Illustrative concentrations for corticosteroids such as hydrocortisone or methylprednisolone acetate are from about 0.2% to about 5.0% by weight.

Additionally, the therapeutic composition may further comprise other enzymes, such as the enzyme lysostaphin for the treatment of any Staphylococcus aureus bacteria present along with the susceptible gram-positive bacteria. Mucolytic peptides, such as lysostaphin, have been suggested to be efficacious in the treatment of S. aureus infections of humans (Schaffner et al., Yale J. Biol. & Med., 39:230 (1967). A recombinant mucolytic bactericidal protein, such as r-lysostaphin, can potentially circumvent problems associated with current antibiotic therapy because of its targeted specificity, low toxicity and possible reduction of biologically active residues.

Methods of application of the therapeutic composition comprising a lytic enzyme/polypeptide(s) include, but are not limited to direct, indirect, carrier and special means or any combination of means. Direct application of the lytic enzyme/polypeptide(s) may be by any suitable means to directly bring the polypeptide in contact with the site of infection or bacterial colonization, such as to the nasal area (for example nasal sprays), dermal or skin applications (for example topical ointments or formulations), suppositories, tampon applications, etc. Nasal applications include for instance nasal sprays, nasal drops, nasal ointments, nasal washes, nasal injections, nasal packings, bronchial sprays and inhalers, or indirectly through use of throat lozenges, mouthwashes or gargles, or through the use of ointments applied to the nasal nares, or the face or any combination of these and similar methods of application. The forms in which the lytic enzyme may be administered include but are not limited to lozenges, troches, candies, injectants, chewing gums, tablets, powders, sprays, liquids, ointments, and aerosols.

When the natural and/or altered lytic enzyme(s)/polypeptide(s) is introduced directly by use of sprays, drops, ointments, washes, injections, packing and inhalers, the enzyme may be in a liquid or gel environment, with the liquid acting as the carrier. A dry anhydrous version of the altered enzyme may be administered by the inhaler and bronchial spray, although a liquid form of delivery can be used.

Compositions for treating topical infections or contaminations comprise an effective amount of at least one lytic enzyme of Table 1, and as described elsewhere in this disclosure. In embodiments, a carrier for delivering at least one lytic enzyme to the infected or contaminated skin, coat, or external surface of a companion animal or livestock. The mode of application for the lytic enzyme includes a number of different types and combinations of carriers which include, but are not limited to an aqueous liquid, an alcohol base liquid, a water soluble gel, a lotion, an ointment, a nonaqueous liquid base, a mineral oil base, a blend of mineral oil and petrolatum, lanolin, liposomes, protein carriers such as serum albumin or gelatin, powdered cellulose carmel, and combinations thereof. A mode of delivery of the carrier containing the therapeutic agent includes, but is not limited to a smear, spray, a time-release patch, a liquid absorbed wipe, and combinations thereof. The lytic enzyme may be applied to a bandage either directly or in one of the other carriers. The bandages may be sold damp or dry, wherein the enzyme is in a lyophilized form on the bandage. This method of application is effective for the treatment of infected skin. The carriers of topical compositions may comprise semi-solid and gel-like vehicles that include a polymer thickener, water, preservatives, active surfactants or emulsifiers, antioxidants, sun screens, and a solvent or mixed solvent system. U.S. Pat. No. 5,863,560 (Osborne) discusses a number of different carrier combinations which can aid in the exposure of the skin to a medicament. Polymer thickeners that may be used include those known to one skilled in the art, such as hydrophilic and hydroalcoholic gelling agents frequently used in the cosmetic and pharmaceutical industries. CARBOPOL is one of numerous cross-linked acrylic acid polymers that are given the general adopted name carbomer. These polymers dissolve in water and form a clear or slightly hazy gel upon neutralization with a caustic material such as sodium hydroxide, potassium hydroxide, triethanolamine, or other amine bases. KLUCEL is a cellulose polymer that is dispersed in water and forms a uniform gel upon complete hydration. Other suitable gelling polymers include hydroxyethylcellulose, cellulose gum, MVE/MA decadiene crosspolymer, PVM/MA copolymer, or a combination thereof.

Compositions comprising lytic enzymes, or their peptide fragments can be directed to the mucosal lining, where, in residence, they kill colonizing disease bacteria. The mucosal lining, as disclosed and described herein, includes, for example, the upper and lower respiratory tract, eye, buccal cavity, nose, rectum, vagina, periodontal pocket, intestines and colon. Due to natural eliminating or cleansing mechanisms of mucosal tissues, conventional dosage forms are not retained at the application site for any significant length of time.

It may be advantageous to have materials which exhibit adhesion to mucosal tissues, to be administered with one or more phage enzymes and other complementary agents over a period of time. Materials having controlled release capability can be used, and the use of sustained release mucoadhesives has received a significant degree of attention. J. R. Robinson (U.S. Pat. No. 4,615,697, incorporated herein by reference) provides a review of the various controlled release polymeric compositions used in mucosal drug delivery. The patent describes a controlled release treatment composition which includes a bioadhesive and an effective amount of a treating agent. Other approaches involving mucoadhesives which are the combination of hydrophilic and hydrophobic materials, are known and are included in the disclosure. The composition includes a freeze-dried polymer mixture formed of the copolymer poly(methyl vinyl ether/maleic anhydride) and gelatin, dispersed in an ointment base, such as mineral oil containing dispersed polyethylene. U.S. Pat. No. 5,413,792 (incorporated herein by reference) discloses paste-like preparations comprising (A) a paste-like base comprising a polyorganosiloxane and a water soluble polymeric material which may be present in a ratio by weight from 3:6 to 6:3, and (B) an active ingredient. U.S. Pat. No. 5,554,380 claims a solid or semisolid bioadherent orally ingestible drug delivery system containing a water-in-oil system having at least two phases. One phase comprises from about 25% to about 75% by volume of an internal hydrophilic phase and the other phase comprises from about 23% to about 75% by volume of an external hydrophobic phase, wherein the external hydrophobic phase is comprised of three components: (a) an emulsifier, (b) a glyceride ester, and (c) a wax material. U.S. Pat. No. 5,942,243 describes some representative release materials useful for administering antibacterial agents, which are incorporated by reference.

Therapeutic or pharmaceutical compositions can also contain polymeric mucoadhesives including a graft copolymer comprising a hydrophilic main chain and hydrophobic graft chains for controlled release of biologically active agents.

The compositions of this application may optionally contain other polymeric materials, such as poly(acrylic acid), poly,-(vinyl pyrrolidone), and sodium carboxymethyl cellulose plasticizers, and other pharmaceutically acceptable excipients in amounts that do not cause deleterious effect upon mucoadhesivity of the composition.

A lytic enzyme/polypeptide(s) of the disclosure may also be administered by any pharmaceutically applicable or acceptable means including topically, orally or parenterally. For example, the lytic enzyme/polypeptide(s) can be administered intramuscularly, intrathecally, subdermally, subcutaneously, or intravenously to treat infections by gram-positive bacteria. In cases where parenteral injection is the chosen mode of administration, an isotonic formulation is may be used. Generally, additives for isotonicity can include sodium chloride, dextrose, mannitol, sorbitol and lactose. In some cases, isotonic solutions such as phosphate buffered saline may be used. Stabilizers include gelatin and albumin. A vasoconstriction agent can be added to the formulation. The pharmaceutical preparations according to this disclosure may be provided sterile and pyrogen free.

For any compound, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. The exact dosage is chosen by the individual physician in view of the patient to be treated. Dosage and administration are adjusted to provide sufficient levels of the active moiety or to maintain the desired effect. Additional factors which may be taken into account include the severity of the disease state, age, weight and gender of the patient; diet, desired duration of treatment, method of administration, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

The effective dosage rates or amounts of the lytic enzyme/polypeptide(s) to be administered parenterally, and the duration of treatment will depend in part on the seriousness of the infection, the weight of the patient, particularly human, the duration of exposure of the recipient to the infectious bacteria, and a variety of a number of other variables. The composition may be administered anywhere from once to several times a day, and may be administered for a short or long term period. The usage may last for days or weeks. Any dosage form employed should provide for a minimum number of units for a minimum amount of time. The concentration of the active units of enzymes believed to provide for an effective amount or dosage of enzymes may be selected as appropriate. The amount of active units per ml and the duration of time of exposure depend on the nature of infection, and the amount of contact the carrier allows the lytic enzyme(s)/polypeptide(s) to have.

### Methods and Assays

The bacterial killing capability, and indeed the significantly broad range of bacterial killing, exhibited by the lysin polypeptide(s) of the disclosure provides for various methods based on the antibacterial effectiveness of the polypeptide(s) of the disclosure. Thus, the present disclosure contemplates antibacterial methods, including methods for killing of gram-positive or Gram-negative bacteria, for reducing a population of gram-positive or Gram-negative bacteria, for treating or alleviating a bacterial infection, for treating a human subject exposed to a pathogenic bacteria, and for treating a human subject at risk for such exposure. The susceptible bacteria are demonstrated herein to include the bacteria from which the phage enzyme(s) of the disclosure are originally derived, Clostridium difficile, as well as various other Clostridium bacterial strains. Methods of treating various conditions are also provided, including methods of prophylactic treatment of Clostridium infections, treatment of Clostridium infections, reducing Clostridium population or carriage, treating lower respiratory infection, treating ear infection, treating ottis media, treating endocarditis, and treating or preventing other local or systemic infections or conditions.

The lysin(s) of the present disclosure demonstrate remarkable capability to kill and effectiveness against bacteria Pseudomonas aeruginosa. The disclosure thus contemplates treatment, decolonization, and/or decontamination of Gram-negative bacteria, cultures or infections or in instances wherein, for example, Klebsiella pneumoniae bacteria are suspected or present. In particular, the disclosure contemplates treatment, decolonization, and/or decontamination of bacteria, cultures or infections or in instances wherein Klebsiella pneumoniae bacteria is suspected, present, or may be present. The same approach applies to other Gram-negative bacteria, including but not limited to Pseudomonas aeruginosa.

Embodiments of this disclosure may also be used to treat gastrointestinal disorders, particularly in a human. For the treatment of a gastrointestinal disorder, such as for colitis, or diarrhea, there should be a continuous intravenous flow of therapeutic agent into the blood stream or oral administration. The concentration of the enzymes for the treatment of colitis and/or diarrhea is dependent upon the bacterial count in the subject.

Also provided is a method for treating Klebsiella pneumoniae infection, carriage or populations comprises treating the infection with a therapeutic agent comprising an effective amount of at least one lytic enzyme(s)/polypeptide(s) of the disclosure, particularly at least one Klebsiella pneumoniae lysins of Table 1. More specifically, lytic enzyme/polypeptide capable of lysing the cell wall of Klebsiella pneumoniae bacterial strains is produced from genetic material from a bacteriophage specific for Klebsiella pneumoniae. In the methods of the disclosure, the lysin polypeptide(s) of the present disclosure, including Klebsiella pneumoniae lysins of Table 1, are useful and capable in prophylactic and treatment methods directed against gram-negative bacteria, particularly Klebsiella pneumoniae infections or bacterial colonization.

The disclosure includes methods of treating or alleviating Klebsiella, including Klebsiella pneumoniae, related infections or conditions, including antibiotic-resistant Klebsiella pneumoniae, particularly including wherein the bacteria or a human subject infected by or exposed to the particular bacteria, or suspected of being exposed or at risk, is contacted with or administered an amount of isolated lysin polypeptide(s) of the disclosure effective to kill the particular bacteria. Thus, one or more of Klebsiella pneumoniae lysins as described herein and within Table 1, including truncations or variants thereof, including such polypeptides as provided herein, in Table 1, is contacted or administered so as to be effective to kill the relevant bacteria or otherwise alleviate or treat the bacterial infection.

The term agent' means any molecule, including polypeptides, antibodies, polynucleotides, chemical compounds and small molecules. In particular the term agent includes compounds such as test compounds, added additional compound(s), or lysin enzyme compounds.

The term ‘agonist’ refers to a ligand that stimulates the receptor the ligand binds to in the broadest sense.

The term ‘assay’ means any process used to measure a specific property of a compound. A screening ‘assay’ means a process used to characterize or select compounds based upon their activity from a collection of compounds.

The term ‘preventing’ or ‘prevention’ refers to a reduction in risk of acquiring or developing a disease or disorder (i.e., causing at least one of the clinical symptoms of the disease not to develop) in a subject that may be exposed to a disease-causing agent, or predisposed to the disease in advance of disease onset.

The term “prophylaxis” is related to and encompassed in the term “prevention”, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

“Effective amount” means an amount of a polypeptide described herein that will elicit the biological or medical response of a subject that is being sought by a medical doctor or other clinician. In particular, with regard to Gram-negative bacterial infections and growth of Gram-negative bacteria, the term “effective amount” is intended to include an amount of the polypeptide that will bring about a biologically meaningful decrease in the amount of or extent of infection of Gram-negative bacteria, including having a bacteriocidal and/or bacteriostatic effect. The phrase “therapeutically effective amount” is used herein to mean an amount sufficient to prevent, reduce by at least about 30 percent, or by at least 50 percent, or by at least 90 percent, a clinically significant change in the growth or amount of infectious bacteria, or other feature of pathology such as for example, elevated fever or white cell count as may attend its presence and activity.

The term “treating” or “treatment” of any disease or infection refers, in one embodiment, to ameliorating the disease or infection (i.e., arresting the disease or growth of the infectious bacteria or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or infection, either physically, (e.g., stabilization of a discernible symptom), physiologically, (e.g., stabilization of a physical parameter), or both. In a further embodiment, “treating” or “treatment” relates to slowing the progression of a disease or reducing an infection.

The phrase “pharmaceutically acceptable” refers to molecular entities and compositions that are physiologically tolerable and do not typically produce an allergic or similar untoward reaction, such as gastric upset, dizziness and the like, when administered to a human.

It is noted that in the context of treatment methods which are carried out in vivo or medical and clinical treatment methods in accordance with the present application and claims, the term subject, patient or individual is intended to refer to a human.

The terms “Gram-negative bacteria”, “Gram-negative” and any variants not specifically listed, may be used herein interchangeably, and as used throughout the present application and claims refer to Gram-negative bacteria which are known and/or can be identified by the presence of certain cell wall and/or cell membrane characteristics and/or by staining with Gram stain. Gram-negative bacteria are known and can readily be identified by those skilled in the art.

The term “bacteriocidal” refers to capable of killing bacterial cells.

The term “bacteriostatic” refers to capable of inhibiting bacterial growth, including inhibiting growing bacterial cells.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to prevent, or to reduce by at least about 30 percent, or at least 50 percent, or at least 90 percent, a clinically significant change in the S phase activity of a target cellular mass, or other feature of pathology such as for example, elevated blood pressure, fever or white cell count as may attend its presence and activity.

One method for treating systemic or bacterial infections parenterally treating the infection with a therapeutic agent comprising an effective amount of one or more lysin polypeptide(s) of the disclosure, including truncations or variants thereof, including such polypeptides as provided herein in Table 1 and an appropriate carrier. A number of other different methods may be used to introduce the lytic enzyme(s)/polypeptide(s). These methods include introducing the lytic enzyme(s)/polypeptide(s) orally, rectally, intravenously, intramuscularly, subcutaneously, intrathecally, and subdermally. One skilled in the art, including medical personnel, will be capable of evaluating and recognizing the most appropriate mode or means of administration, given the nature and extent of the bacterial condition and the strain or type of bacteria involved or suspected.

Infections may be also be treated by injecting into the infected tissue of the human patient a therapeutic agent comprising the appropriate lytic enzyme(s)/polypeptide(s) and a carrier for the enzyme. The carrier may be comprised of distilled water, a saline solution, a buffered solution, albumin, a serum, or any combinations thereof. More specifically, solutions for infusion or injection may be prepared in a conventional manner, e.g. with the addition of preservatives such as p-hydroxybenzoates or stabilizers such as alkali metal salts of ethylene-diamine tetraacetic acid, which may then be transferred into fusion vessels, injection vials or ampules. Alternatively, the compound for injection may be lyophilized either with or without the other ingredients and be solubilized in a buffered solution or distilled water, as appropriate, at the time of use. Non-aqueous vehicles such as fixed oils, liposomes, and ethyl oleate are also useful herein. Other phage associated lytic enzymes, along with a holin protein, may be included in the composition.

Various methods of treatment are provided for using a lytic enzyme/polypeptide(s), such as those of Table 1 and as otherwise described herein , as a prophylactic treatment for eliminating or reducing the carriage of susceptible bacteria, preventing those humans who have been exposed to others who have the symptoms of an infection from getting sick, or as a therapeutic treatment for those who have already become ill from the infection. Thus, the polypeptides of the disclosure may be used to eliminate, characterize, or identify the relevant and susceptible bacteria.

Thus, a diagnostic method of the present disclosure may comprise examining a cellular sample or medium for the purpose of determining whether it contains susceptible bacteria, or whether the bacteria in the sample or medium are susceptible by means of an assay including an effective amount of one or more lysin polypeptide(s). A fluid, food, medical device, composition or other such sample which will come in contact with a subject or patient may be examined for susceptible bacteria or may be eliminated of relevant bacteria. In one such aspect a fluid, food, medical device, composition or other such sample may be sterilized or otherwise treated to eliminate or remove any potential relevant bacteria by incubation with or exposure to one or more lytic polypeptide(s) of the disclosure.

The procedures and their application are all familiar to those skilled in the art in view of the present disclosure, and accordingly may be utilized within the scope of the present disclosure. In one embodiment, the lytic polypeptide(s) of the disclosure complex(es) with or otherwise binds or associates with relevant or susceptible bacteria in a sample and one member of the complex is labeled with a detectable label. The fact that a complex has formed and, if desired, the amount thereof, can be determined by known methods applicable to the detection of labels. The labels most commonly employed for these studies are radioactive elements, enzymes, chemicals which fluoresce when exposed to ultraviolet light, and others.

The disclosure may be better understood by reference to the following non-limiting Examples, which are provided as exemplary of the disclosure. The following examples are presented in order to more fully illustrate the embodiments of the disclosure and should in no way be construed, however, as limiting the broad scope of the disclosure.

### EXAMPLES

### Example 1

### Bacterial Strains and Growth Conditions

E. coli strains DH5α and BL21 (DE3) were obtained from Thermo Fisher Scientific (Waltham, Mass., USA). Klebsiella species 1_1_55 (catalog number: HM-44) and K. pneumoniae strain BIDMC-11 (catalog number: NR-41927) were obtained from BEI Resources (Manassas, Va., USA). The K. pneumoniae strains PCI 602 (ATCC#10031) and K6 (ATCC#700603) were obtained from ATCC (Manassas, Va., USA). All bacterial cultures were grown in Lysogeny broth (LB) at 37° C., shaking at 200 rpm.

Cloning of Candidate Klebsiella Pneumoniae and Enterobacter Lysins in to a 6xHis-Tag pET Expression System

The lysin genes were ordered from Genewiz (South Plainfield, N.J., USA) and amplified by PCR using a High-Fidelity Phusion polymerase (New England Biolabs, Ipswich, Mass., USA). An exception is PlyKp105. Lysin PlyKp105 was amplified from the genome of Klebsiella pneumoniae NR-41923 using primers 493-F (“cccgtcgacatggctaacctgaaaacgaaactc”) (SEQ ID NO:59) and 493-R (“cccgcggccgctcattcatctatcccccaacatg”) (SEQ ID NO:60). Lysin amplicons were purified with a QlAquick PCR Purification Kit (Qiagen GmbH, Hilden, Germany) and ligated in to a pET vector linking inserts to an N-terminal 6xHis-tag, creating pET^{PlyKp01-PlyKp86}. The twelve pET^{PlyKp01-PlyKp86} plasmids were mixed with RbCl-competent DH5α cells, incubated on ice for lhr and submerged in a 42° C. water bath for 1 min. Heat-shocked cells were incubated in LB for 1 hr before inoculation on LB agar supplemented with 100 μg/ml ampicillin. Colonies carrying pET^{PlyKp01-PlyKp86} were identified by PCR and harvested of their plasmids with a QIAprep Spin Miniprep Kit (Qiagen GmbH). The plasmids were sequenced by Genewiz and then used to transform E. coli BL21 (DE3) by heat-shock as described. In some embodiments, lysins were cloned into a pBAD24-based plasmid that was engineered to contain SalI and NotI restriction sites. All cloning procedures to create these plasmids were similar for those described for the pET vector described above.

### Example 2. Recombinant Expression and Purification of Candidate Lysins

Overnight cultures of BL21 (DE3) with pET^{PlyKp01-PlyKp86} were diluted 1:100 in 400 ml LB supplemented with 100 μg/ml ampicillin. Lysin expression was induced with 10 μM IPTG at log phase (OD₆₀₀=0.4-0.6) for 4 hrs, then moved to 4° C. at 70 rpm overnight. Induced cultures were centrifuged at 5,000 rpm for 15 min and the pellets were re-suspended in purification buffer (0.5 M NaCl, 10% glycerol and 20 mM Tris at pH 7.9). Re-suspended cells were lysed with an EmulsiFLex-C5 homogenizer (Avestin Inc., Ottawa, Ontario, Canada) and debris was pelleted and removed by centrifugation at 12,000 rpm for 15 min, twice. The lysates were filtered through 0.2 μm Nalgene™ filters (Thermo Fisher Scientific) before addition to columns loaded with HisPur™ Ni-NTA resin (Thermo Fisher Scientific), calibrated with purification buffer. The columns were washed six times with 5 column volumes (CVs) of purification buffer supplemented with 0-30 mM imidazole, and lysins were eluted with 25 ml 150 mM imidazole. The elution buffer was supplemented with 10 mM EDTA and 1 mM DTT, and the 6xHis-tag was cleaved by overnight incubation with Human Rhinovirus 3C Protease (produced by our lab), at 4° C. and shaking at 70 rpm. The lysin buffer was exchanged by overnight dialysis in a Spectra/Por® 3Dialysis Membrane (Spectrum Laboratories Inc., Rancho Dominguez, Calif., USA) in 3.51 PBS. The protein concentration was increased by reducing the sample volume to 1-2 ml by centrifugation in a Amicon Ultra 10 kDa tube (Merck Millipore Ltd, Cork, Ireland) at 4,000 rpm, and then measured by a NanoDrop-1000 Spectrophotometer (Thermo Fisher Scientific). The sample purity was determined by loading the lysins on an SDS-PAGE and staining the gel with Coomassie blue. Table 2 shows a summary of the total protein yield and an estimation of purity of 12 K. pneumoniae lysins. In some embodiments lysins cloned into a pBAD24-based vector were used as a means to assess lysin activity. Induction of lysin was performed using 0.2% arabinose, and procedures were carried out as described above up to the production of crude lysate. Crude lysate was used to assess peptidoglycan hydrolysis activity on a soft agar overlay.

TABLE 2

| Lysin | Total yield (mg) | Purity (%) |
|---|---|---|
| PlyKp01 | 0.6 | 25 |
| PlyKp06 | 3.7 | 50 |
| PlyKp09 | 1.1 | 40 |
| PlyKp10 | 1.6 | 50 |
| PlyKp13 | 1.2 | 40 |
| PlyKp16 | 1.1 | 30 |
| PlyKp17 | 2.7 | 50 |
| PlyKp57 | 0.3 | <10 |
| PlyKp61 | 0.1 | <10 |
| PlyKp68 | 0.9 | 50 |
| PlyKp75 | 1.1 | 40 |
| PlyKp86 | 0.9 | 40 |

### Example 3. Screening for Lytic Activity on Peptidoglycan

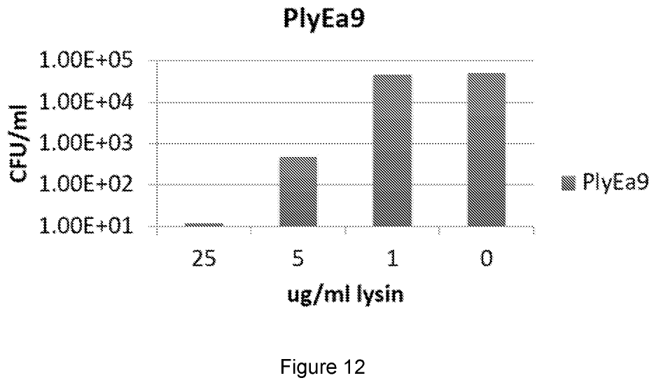

To create peptidoglycan agar plates, 0.8% agarose and 0.1% freeze-dried Micrococcus luteus were dissolved in 30 mM HEPES at pH 7.4. The mixture was autoclaved until completely dissolved, then poured in to Petri dishes and allowed to solidify. 10μ1 of purified lysins were added to the peptidoglycan agar, and plates were incubated at 37° C. for 1 hr. Clearing zones around lysins were seen with all 12 Klebsiella PlyF307 homologues, indicating lytic activity. Additional lysins were screened (FIG. 12).

### Example 4. Lytic Killing Assays of Klebsiella Species

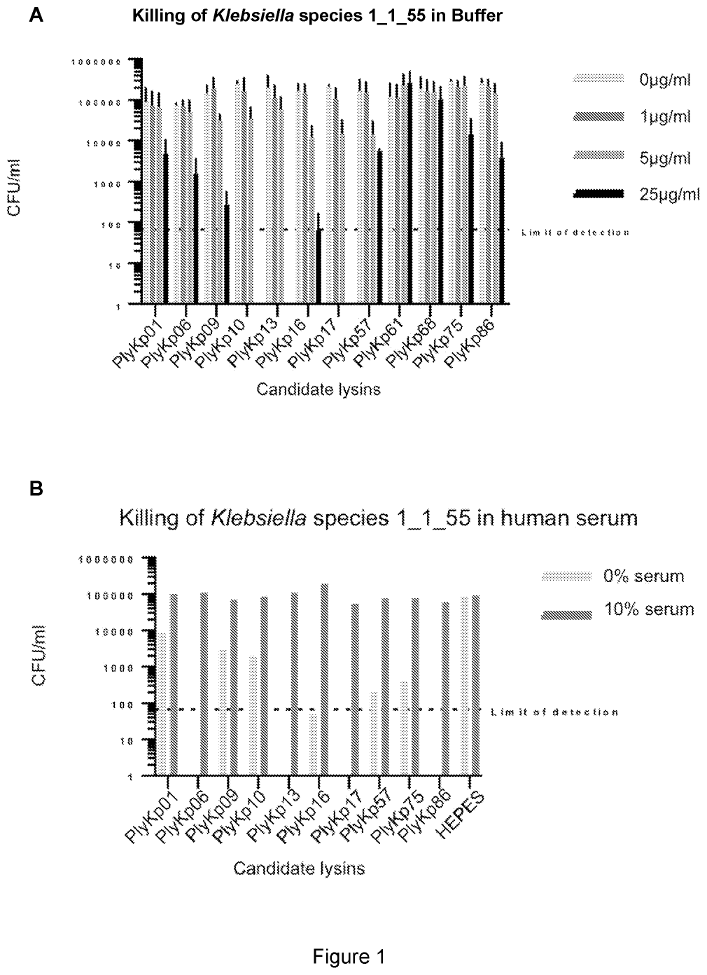

To reduce the risk for lab personnel, a less virulent Klebsiella species (1_1_55) closely related to K. pneumoniae was used in the candidate screenings of killing activity. K. pneumoniae strains PCI 602, K6 and BIDMC-11 were used to further investigate the activity of PlyKp17 (with 1_1_55 as positive control). In all such killing conditions, ˜10⁵ cells/ml were incubated with lysins for 1 hr at 37° C., shaking at 200 rpm in a 96-welled u-bottomed plate. Overnight cultures were diluted 1:50 and grown to log-phase (OD₆₀₀=0.4-0.6≈10⁶ cells/ml). Bacteria were centrifuged for 10 min at 4,000 rpm and the pellets were washed twice with 30 mM HEPES at pH 7.4. In all HEPES assays, bacteria were incubated with lysins at 0 μg/ml, 1 μg/ml, 5 μg/ml and 25 μg/ml. For the preliminary screening of killing efficiency in human serum, bacteria in HEPES supplemented with 10% serum (Sigma-Aldrich, St. Louis, Mo., USA) were incubated with the highest possible volume of lysin (50 μl). After incubation, 15 μl of HEPES cultures and 20 μl of serum cultures were serially diluted 1:1, 1:10, 1:100, 1:1000, streaked in single lines on LB agar plates and incubated overnight at 37° C. In the morning, colonies were counted and used to estimate CFU/ml. FIG. 1 shows at 25 μg/ml, PlyKp10, PlyKp13 and PlyKp17 decreased 1_1_55 CFU/ml below the limit of detection (<67 CFU/ml), and all but PlyKp61 and PlyKp68 reduced CFU/ml to some extent (n=2) (FIG. 1A). Next, we screened if the active lysins retained killing activity in human serum. For this, 1_1_55 in 10% serum were incubated with the maximum possible volume of purified lysins. No reduction of CFU/ml was observed for any lysin (n=1) (FIG. 1B).

Killing of different Klebsiella pneumoniae strains by PlyKp17

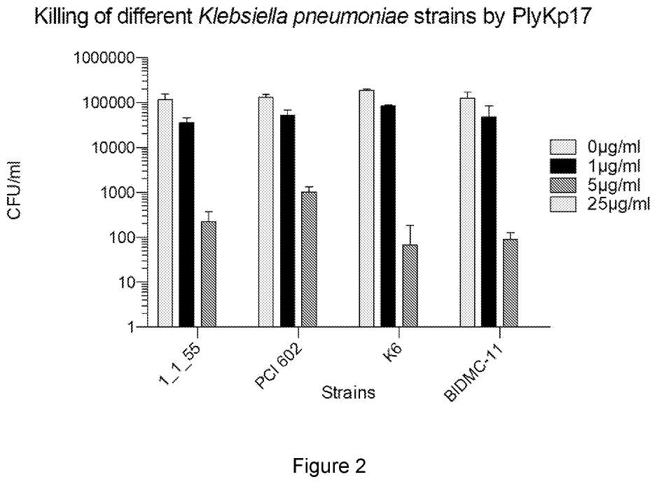

PlyKp17 was deemed to be the most promising lysin candidate of the Klebsiella PlyF307 homologues, considering both the purification yield and results from activity screenings. While the preliminary killing screenings were performed on Klebsiella species 1_1_55, we now wanted to investigate the activity of PlyKp17 on different K. pneumoniae strains. FIG. 2 shows the results from PlyKp17 incubated with three clinical strains, of which one was antibiotic sensitive, one was producing extended spectrum β-lactamases (ESBLs) and one was carbapenem-resistant. PlyKp17 proved to be highly active in HEPES buffer at pH 7.4, significantly reducing CFU/ml of all strains at 5 μg/ml reduction) and further decreasing them below the limit of detection (<67 CFU/ml, a 5-log reduction) at 25 μg/ml (n=3).

### Example 5. Testing the PlyKp17 Activity on Micrococcus Luteus in Human Serum

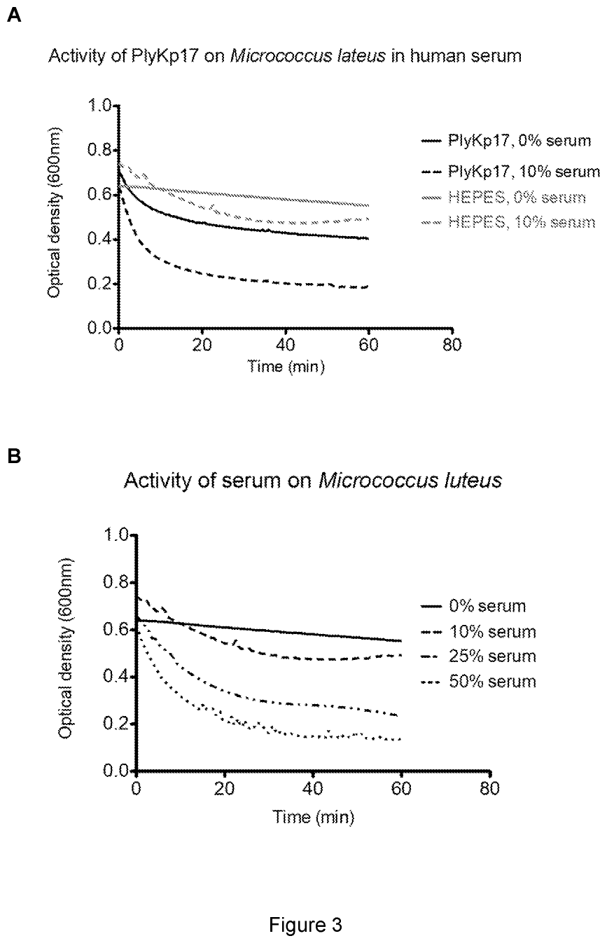

The highest possible volume (50 μl) of PlyKp17 was mixed with human serum and freeze-dried M. luteus dissolved in 30 mM HEPES, to yield a final serum concentration of 0%, 10%, 25% or 50% and OD₆₀₀≈0.7. Immediately after the addition of bacteria, the OD₆₀₀ was measured once every 30 s for lhr by a SpectraMax M5 Microplate Reader (Molecular Devices, San Jose, Calif., USA). M. luteus is a Gram-positive with a very thick, exposed cell wall and lytic activity can be measured as reductions in M. luteus OD₆₀₀ over time. FIG. 3 shows results that at 10% serum, PLYKP17 has an additive effect with serum components to hydrolyse the M. luteus peptidoglycan more efficiently than with only serum or lysin alone (FIG. 3A). The serum itself drastically reduced OD₆₀₀ at percentages above 10%, making it difficult to estimate the activity of the PlyKp17 at those concentrations (FIG. 3B).

### Example 6. Recombinant Expression and Two-Step Affinity Chromatography Purification of PlyKp17 and PlyKp17-RI18

Using the common cloning methods previously described, PlyKp17 was cloned into two new pET vectors: one linking it to a N-terminal GST/6xHis-tag and one to both an N-terminal GST/6xHis-tag and a C-terminal RI18 (a membrane-disrupting AMP). In both cases the lysin is separated from the purification tags by a cleavable 3C site. Overnight cultures of BL21 (DE3) with pET^{PlyKp17} or pET^{PlyKp17-RI18} were diluted 1:100 in 800 ml LB supplemented with 100 μg/ml ampicillin. Protein expression was induced with 0.2 mM IPTG at OD₆₀₀≈1.2 for 4 hrs, then moved to 4° C. at 70 rpm overnight. Induced cultures were centrifuged at 5,000 rpm for 15 min and the pellets were re-suspended in purification buffer (0.5 M NaCl, 10% glycerol and 20 mM Tris at pH 7.9). Re-suspended cells were lysed with an EmulsiFLex-C5 homogenizer and debris was pelleted and removed by centrifugation at 15,000 rpm for 30 min. The lysates were filtered through 0.2 μm Nalgene™ filters before addition to columns loaded with HisPur™ Ni-NTA resin calibrated with purification buffer. The columns were washed six times with 5 CVs of purification buffer supplemented with 0-20 mM imidazole, and proteins were eluted with 25 ml 200 mM imidazole. The eluted protein solutions were added to a Glutathione Sepharose column (Sigma-Aldrich) calibrated with purification buffer. The column was washed once with 5 CVs purification buffer and once with 5 CVs wash buffer (150 mM NaCl, 150mM Tris-HCl, 10% glycerol). The proteins were eluted with 50 ml wash buffer supplemented with 10 mM reduced L-Glutathione. The elution buffer was supplemented with 1 mM EDTA and 1 mM DTT, and the GST-6xHis-tag was cleaved by overnight incubation with Human Rhinovirus 3C Protease, at 4° C. and shaking at 100 rpm. The lysin buffer was exchanged by overnight dialysis in a Spectra/Por® 3Dialysis Membrane in 3.5 l PBS. The protein concentrations were increased by reducing the sample volume to 2 ml by centrifugation in Amicon Ultra 3 kDa tube at 4,000 rpm, and then measured by a NanoDrop-1000 Spectrophotometer. The purities were determined by loading the concentrated samples on an SDS-PAGE and staining the gel with Coomassie blue.

### Example 7. Killing of Klebsiella Pneumoniae in Human Serum by PlyKp17-RI18

To investigate if the PlyKp17-RI18 fusion improved antimicrobial activity in serum, K. pneumoniae was incubated with 100 μg/ml PlyKp17-RI18for 1 hr in up to 50% human serum (n=3).

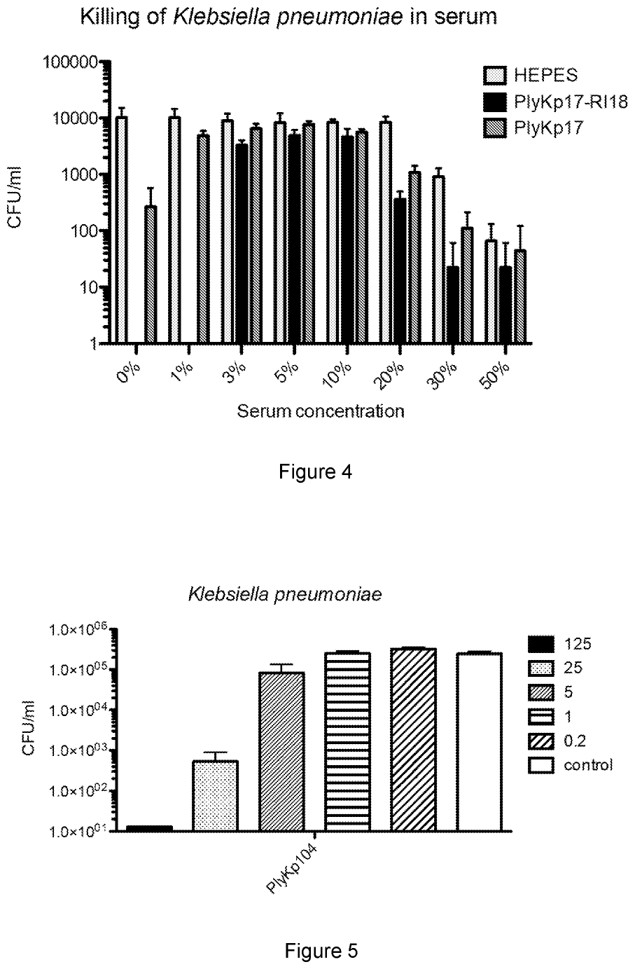

An overnight culture of PCI 602 was diluted 1:50 and grown to log-phase (OD₆₀₀=0.4-0.6≈10⁶ cells/ml). Bacteria were centrifuged for 10 min at 4,000 rpm and pellets were washed twice with 30 mM HEPES at pH 7.4. Roughly 10⁴ cells/ml in 0-50% human serum were incubated with 100 μg/ml of PlyKp17-RI18 for 1 hr at 37° C., shaking at 200 rpm in a 96-welled u-bottomed plate. PlyKp17 purified by two-step affinity chromatography was used as positive control for 0% serum and negative control for higher serum concentrations, and HEPES was used as general negative control. After incubation, 15 μl of cultures were serially diluted 1:1, 1:10, streaked in single lines on LB agar plates and incubated overnight at 37° C. In the morning, colonies were counted and used to estimate CFU/ml. FIG. 4 shows the preliminary results that at 0% and 1% serum, it decreased the CFU/ml below the limit of detection (67 CFU/ml) but at higher concentrations no substantial reduction was observed.

### Example 8. Additional Klebsiella Lysins

Additional Klebsiella lysins were investigated. PlyKp104, from in silico analysis utilizing the reference lysin PlyPa101, and PlyKp105, amplified from a prophage in the genome of Klebsiella pneumoniae strain NR-41923 prophage genome.

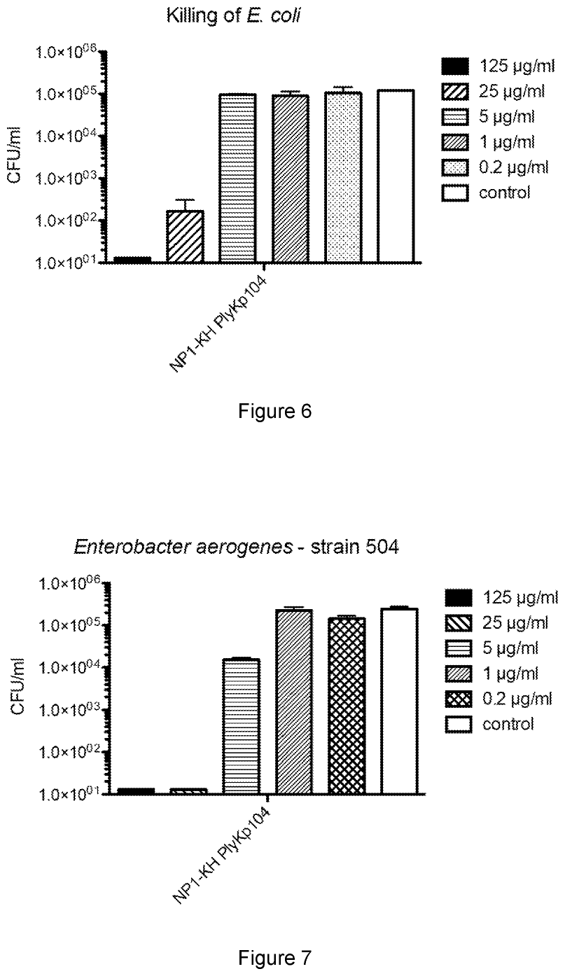

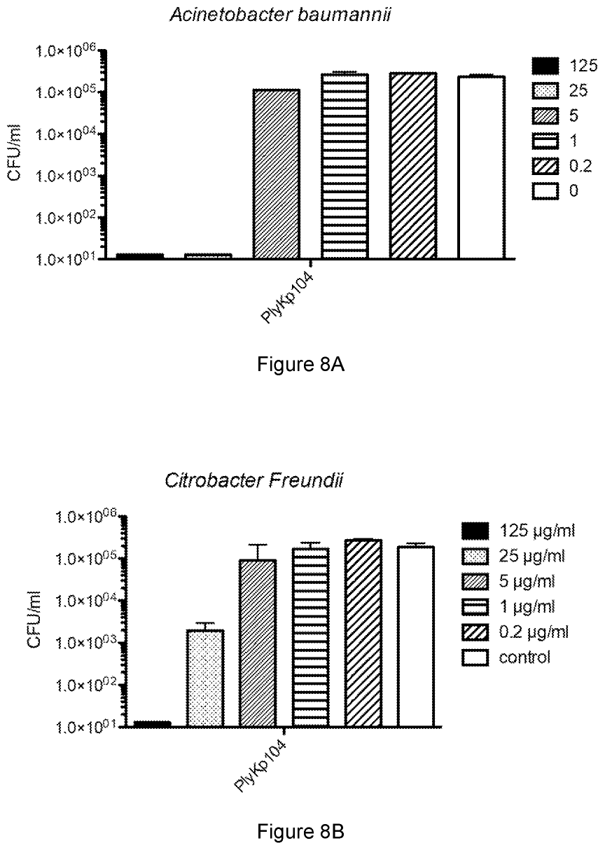

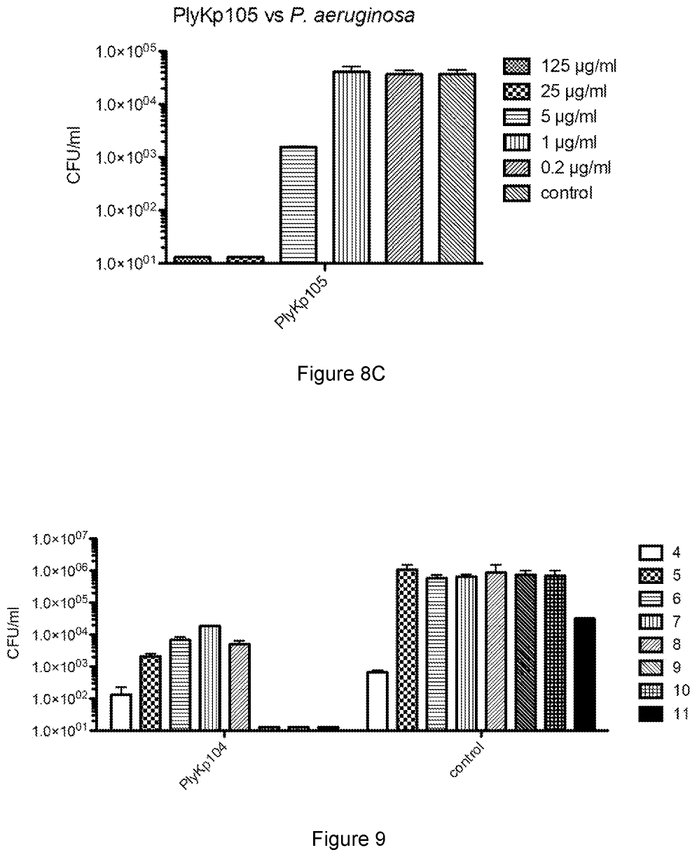

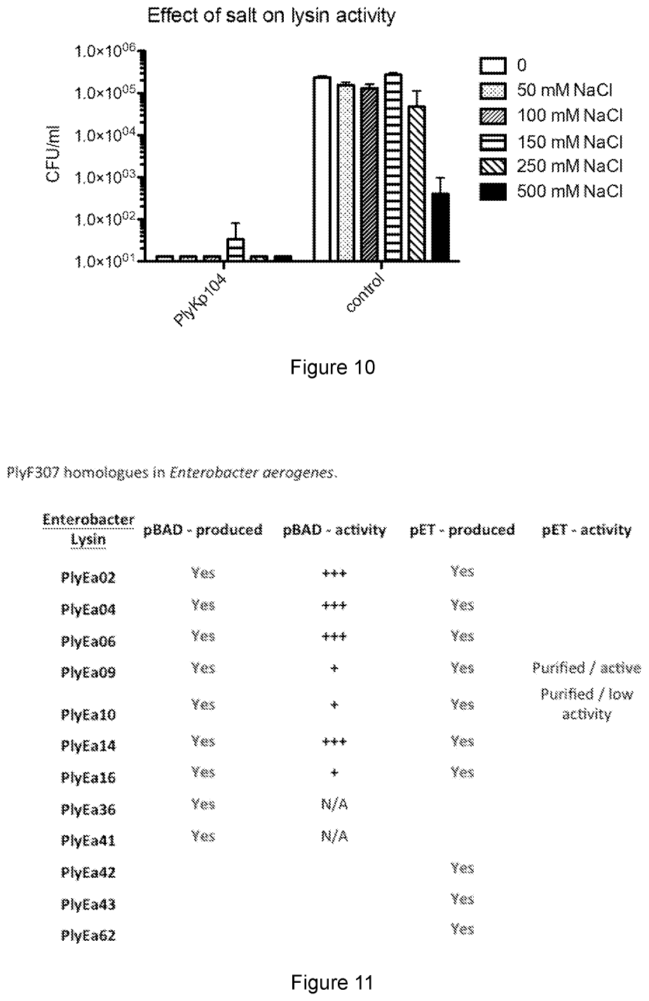

PlyKP104 demonstrated robust killing activity of several Gram-negative species including Klebsiella pneumoniae (FIG. 5), Escherichia coli (FIG. 6), Enterobacter aerogenes (FIG. 7), and Acinetobacter baumannii (FIG. 8A), Citrobacter freundii (FIG. 8B). PlyKp105 was demonstrated to be catalytically active against P. aeruginosa (FIG. 8C). PlyKP104 demonstrated robust killing activity of Pseudomonas aeruginosa under a wide range of pH conditions (FIG. 9) and salt concentrations (FIG. 10).

### Example 9. Enterobacter Lysins

Enterobacter PlyF307 homologues were identified, purified, and analyzed as described above for the Klebsiella enzymes. Analogous methods were used to find, produce plasmids, express, purify and test the lysins. Many Enterobacter lysins were initially produced as pBAD24-based plasmids as described above. These constructs were used to screen for lytic activity by an overlay assay following induction of protein expression with 0.2% arabinose, and permeabilization of the cells with chloroform vapor. The summary of the plasmids produced, as well as lysis results by an overlay assay are shown in FIG. 11. Killing assay of Enterobacter aerogenes by pure PlyEa09 is presented in FIG. 12.

### Example 11. Antimicrobial Peptide (AMP) Fusions to Lysins

We have found that fusion of AMP to a lysin in the N or C terminus can improve its activity, and specifically activity in serum.

A non-exclusive list of AMPs that can be fused at the N or C terminus of lysins include: LALF, LL-37, RI-18, WLBU, RP-1, Pexiganan. Further, AMPs or a portion of the peptide could be used. In some instances enzymes were annotated in their formal name (i.e. PlyKp01 for Klebsiella enzymes or PlyEa02 for Enterobacter enzymes), and in other instance these same enzymes have been referred to by shorthand names (i.e. KL01 for Klebsiella lysins and EL02 for Enterobacter lysins). These different names are interchangeable names for the same molecules. The same approach refers to lysins that are specific for other Gram-negative bacteria described herein, such as Pseudomonas aeruginosa.

TABLE 1

> PlyKp01 | LOCUS WP_032191494
Memsnnginmlkgfegcrlaayqdsvgvwtigygwtqpvng
vpvgkgmtitqdtadsllrsglvqyekgvtglvkvtinqnq
fdalvdfaynlgvkalegstllkklnagdyagaaaefpkwn
kaggkvlpglvkrreaertlfla
(SEQ ID NO: 1)
> PlyKp06 | LOCUS WP_063963664
Mqtsekgislikefegcklnayqdsvgvwtigygwtqpvdg
Kpiragmtikqetaerllktglvsyesdvsrlvkvgltqgq
Fdalvsftynlgarslststllrklnagdyagaadeflrwn
Kaggkilngltrrreaeralfls
(SEQ ID NO: 2)
> PlyKp09 | LOCUS WP_042714022
Manqpqhtgdagvaliksfeglrlekyrdavgkwtigyghl
Ilpnenfprpiteaeadallrkdlqtsergvhrlvtvdldq
Dqfdalvsftfnlgagnlqsstllkllnqgeytqaadqflr
Wnkaggrvlpgltrrreaeralflqag
(SEQ ID NO: 3)
> PlyKp10 | LOCUS WP_048329977
Mqtspegialikgfegcrltaypdpgtggvpwtigygwtlp
Idgkpvrpgmtidqvtadrllktglvsyesdvlkivkvkln
Qnqfdalvsfaynvgsralststllkklnagdikgaadefl
Rwnkaggkvlngltrrreaeralfls
(SEQ ID NO: 4)
> PlyKp13 | LOCUS WP_019725080
Mqisnngialikrfegcrltaypdpgtggdpwtigygwtg
Kvdgkpirpgmkideatadrllrtgwsfdqavskmlkvtv
Tqnqydalvslaynigtralststlmkklnagdvkgaade
Flrwnrsggkvmagltnrrkaerevfls
(SEQ ID NO: 5)
> PlyKp16 | LOCUS WP_068987105
Mqisnngialikrfegcrltaypdpgtgggpwtigygwtg
Kvdgkpikpgmkiddatadrllrtgvvsfdqavskmlkvs
Vtqnqydalvslaynigtralststlmkklnagdvkgaad
Aflswnrsggkvmagltnrrkaerevfls
(SEQ ID NO: 6)
> PlyKp17 | LOCUS WP_044067377
Mqisdngialikgfegcrltaypdpgtggdpwtigfgwtg
Kvdgkpikpgmkiddatadrllrtgvvsfdlavskmlkvs
Vtqnqydalvslaynigtralststlmkklnagdvkgaad
Eflrwnksggkamsgltnrrkaerevflsktrgsyelsh
(SEQ ID NO: 7)
> PlyKp57 | LOCUS WP_048333081
Mnptlmkligaiaggsgaiviasvmlgnadglegrryyay
Qdvvgvwtvcdghtgtdirrghrytdrecdnllkadlrkv
Asaidplikvsipdptraalysftynvgsgafasstllkk
Lnagdvpgackelqrwtyaggkqwkglisrreierevclw
gqk
(SEQ ID NO: 8)
> PlyKp61 | LOCUS SAT14280
Mvmspklrnsvlaavgggaiaiasalitgptgndglegvr
Ykpyqdvvgvwtvcyghtgkdimlgktytesecrallnkd
Lnivarqinpyiqkpipetmrgalysfaynvgagnlqtst
Llrkinqgdqkgacdqlrrwtyakgkqwkglvtrreiere
vclwgqk
(SEQ ID NO: 9)
> PlyKp68 | LOCUS WP_048264621
Mrissngvvrlkgeegerlsayldsrgiptigvghtgtvd
Gkpwigmvisqnkstelllqdiqwvekainssvktpltqn
Qydalcslvfnigatafygstvlkrvnqkdytaaadaflm
Wkkagkdqeillprrrreralfls
(SEQ ID NO: 10)
> PlyKp75 | LOCUS WP_024622713
Mnptlmkligaiaggsgaiaiasvmlgnadglegrryyay
Qdwgvwtvcdghtgtdirrghrytdrecdsllkadlrkva
Saidpiikvripdptraalysftynvgsgafasstllkkl
Nagdvpgackelqrwtyaggkqwkglitrreierevcewg
qk (SEQ ID NO: 11)
> PlyKp86 | LOCUS WP_057216474
Mnptlmkligaiaggsgaiaiasvmlgnadglegrryyay
Qdwgvwtvcdghtgtdirrghrytdrecdnllkadlrkva
saidpiikvrlpaptraalysftynvgsgafasstllkkl
nagdvpgackelqrwmyaggkqwkglitrreierevcewg
qk
(SEQ ID NO: 12)
> PlyEa02 | LOCUS WP_063159646
Mqtsekgialikefegckltayqdsvgvwtigygwthpvd
Gkpiragmtikqetaerllktglvsyecdvsrlvkvgltq
gqfdal
vsftynlgarslststllrklnagdyagaadeflrwnkag
gkvlngltrrreaeralfls
(SEQ ID NO: 13)
> PlyEa04 | LOCUS WP_058675961
Mqisdegialikgfegcrltaypdpgtggapwtigygwtl
Pvdgkpvrpgmtidqatadrllkiglvgyendvlkivkvk
Ltqgqfdalvsfaynigsralststllkklnagdikdaad
Eflrwnkaggkvlngltrrreaeralfls
(SEQ ID NO: 14)
> PlyEa06 | LOCUS WP_045381882
Mqvsdngivflkneegekltgypdsrgiptigvghtgkvn
Gvpvsvgmkitseqssellkddlswvedsianyvksplnq
Nqydalcsfifnigapafegstmlkllnksdyvgasgefp
Kwkragndpdillprrmreqalfls
(SEQ ID NO: 15)
> PlyEa09 | LOCUS WP_059444542
Mqissngitklkreegerlkaypdsrgiptigvghtgnvd
Gkpvtlgmtitsdkssellkadlrwvedaisslvrvpltq
Nqydalcslifnigksafagstvlrqlnlknyqaaadafl
mwkkagkdteillprrqreralfls
(SEQ ID NO: 16)
> PlyEa10 | LOCUS WP_047076801
Mnptlmkligaiaggsgaiaiasvmlgnadglegrryyay
Qdvvgvwtvcdghtgsdirrghrysdkecdnllksdlrkv
Anaidplikvripdptraalysftynvgsgafasstllkk
Lnagdvpgackelqrwtyaggkqwkglitrreielevcew
Gqk
(SEQ ID NO: 17)
> PlyEal4 | LOCUS WP_042895492
Mnqplrkyvlsavgggaiaiasalitgptgndglegvryq
Pyqdwgvwtvcyghtgkdimlgntytksecdalldkdlnt
Varqinpyikkpipetmrgalysfaynvgagsfqtstllr
Kinqgdskgaceqlrvwiyagkkvwkglvtrreierevcl
wgqk
(SEQ ID NO: 18)
> PlyEal6 | LOCUS WP_063447397
Mnpsivkrclvgavlaiaatlpgfqslhtsveglkliad
Yegcrlqpyqcsagvwtdgigntsgvvpgktiterqaaq
Glitnvlrveraldkcvaqpmpqkvydawsfafnvgtgn
Acsstlvkllnqrrwadachqlprwvyvkgvfnqgldnr
raremawclkga
(SEQ ID NO: 19)
> PlyEa36 | LOCUS KZQ44728
Mamspalrnsivaalgtgaigiatvmvsgksglegrehy
Pykdivgivtvcdgytgsdivwgkyysdkecdaltrkdm
Triaaqvnphikvpttetqraaiysfaynvgstaainst
Llkklnskdysgacselkrwvyaggkkwkglmnrrdvey
evctwsqk
(SEQ ID NO: 20)
> PlyEa41 | LOCUS WP_063411204
mssivkrcsvaavlalaallpdfrllhtspdglaliadl
egcrlapyqcsagvwtsgightagwpkrditereaaanl
vadvlnterrlavcvpvtmpqpvydalvsfsfnvgtgaa
crstlvsyikrhqwwqacdqlsrwvyvngerstglenrr
qrerayclkgvk
(SEQ ID NO: 21)
> PlyEa42 | LOCUS WP_049056838
Mtnkvkfsaamlallaagatapelfdqfmsekegnalva
Vvdpggvwslchgvifidgkrvvkgmtatesqcrkvnai
Erdkalswvdminvpltepqkvgiasfcpynigpgkcfp
Stfykrinagdrkgaceairwwikdggkdcrirsnncyg
qvtrrdqesaltcwgidq
(SEQ ID NO. 22)
> PlyEa43 | LOCUS WP_058650108
msnkakfsaamlvllaagasapvlfdqfigeregnslta
vidpggvwsicrgvtridgrpwkgmnltqsqcdhynaie
rdkalawvqknvhvpltepqkvgiasfcpynigpgkcfp
stfyrklnagdrkgacaeirrwifdggrdcrltkgqang
cygqvdrrdqesaltcwglye
(SEQ ID NO. 23)
> PlyEa62 | LOCUS WP_059304620
Maslktklsaamlgliaagasaptlmdqfldekegnslt
Ayrdgsqgiwticrgatridgkpvtqgmkltqakcdevn
Dierdkalawvdrnirvpltppqkvgiasfcpynigpgk
Cfpstfyqrinagdrkgaceairwwikdggkdcrirsnn
cygqvtrrdqesaltcwgidq
(SEQ ID NO: 24)
>PlyKp104
MAWGAKVSKEFKLKVIEVCERLEINPDYLMSCMAFETGE
TFSPNVRNPNGSATGLIQFMSNTARSLGTTTNELADMTS
VEQMDYVEKYFKPYAGKIKTIEDVYMVIFCPRAVGKPDS
YILYDEGRSYNDNKGLDLNKDNAITKYEAGFKVREKLKL
GMKEGYRG
(SEQ ID NO: 25)
>PlyKp105 (internal name KLB-493-1)
MANLKTKLSSAMLALIAAGASAPVLMDQFLNEKEGKSLT
SYRDGAGIWTICRGVTQVDGRPVTQGMKLTQAKCDQVNA
VERNKALAWVDQNVRVPLTPPQKVGIASFCPYNIGPGKC
FPSTFYRKLNAGDRKGACAEIRRWIFDGGKDCRVRSNNC
YGQVSRRDQESALACWGIDE
(SEQ ID NO: 26)
>PlyKp17/LALF (lysin + antimicrobial
peptide)
MQISDNGIALUCGFEGCRLTAYPDPGTGGDPWTIGFGWT
GKVDGKPIKPGMKIDDATADRLLRTGVVSFDLAVSKMLK
VSVTQNQYDALVSLAYNIGTRALSTSTLMKKLNAGDVKG
AADEFLRWNKSGGKAMSGLTNRRKAEREVFLSKTRGSYE
LSHGTGGGSGGGSGGGDHECHYRIKPTFRRLKWKYKGKF
WCPS
(SEQ ID NO: 27)
>PlyKp17/LL-37 (lysin + antimicrobial
peptide)
MQISDNGIALIKGFEGCRLTAYPDPGTGGDPWTIGFGWT
GKVDGKPIKPGMKIDDATADRLLRTGVVSFDLAVSKMLK
VSVTQNQYDALVSLAYNIGTRALSTSTLMKKLNAGDVKG
AADEFLRWNKSGGKAMSGLTNRRKAEREVFLSKTRGSYE
LSHGTGGGSGGGSGGGLLGDFFRKSKEKIGKEFKRIVQR
IKDFLRNLVPRTES
(SEQ ID NO: 28)
>PlyKp17/RI-18 (lysin + antimicrobial
peptide)
MQISDNGIALIKGFEGCRLTAYPDPGTGGDPWTIGFGWT
GKVDGKPIKPGMKIDDATADRLLRTGVVSFDLAVSKMLK
VSVTQNQYDALVSLAYNIGTRALSTSTLMKKLNAGDVKG
AADEFLRWNKSGGKAMSGLTNRRKAEREVFLSKTRGSYE
LSHGTGGGSGGGSGGGRKKTRKRLKKIGKVLKWI
(SEQ ID NO: 29)
>PlyPa101
MKLAWGKKVDQAFRDKVFAICDGFKWNRETHASWLMSCM
AFESGETFSPSVRNAAGSGATGLIQFMPRTAQGLGTSTA
ELAAMSAVDQLDYVQKYFRPYASRIGTLSDMYMAILMPK
FVGQPEDSVLFLDPKISYRQNAGLDANRDGKITKAEAAS
KVRAKFDKGMLDRFALEL
(SEQ ID NO: 63)
>PlyPa103
MAWSAKVSQAFCDRVIWIAASLGMPADGADWLMACIAWE
TGETFSPSVRNGAGSGATGLIQFMPATARGLGTTTDELA
RMTPEQQLDYVYRYFLPYRGRLKSLADTYMAILWPAGIG
RALDWALWDSTSRPTTYRQNAGLDINRDGVITKAEAAAK
VQAKLDRGLQPQFRRAAA
(SEQ ID NO: 64)
>PlyPa102
MKITKDVLITGTGCTTDRAIKWLDDVQAAMDKFHIESPR
AIAAYLANIGVESGGLVSLVENLNYSAQGLANTWPRRYA
VDPRVRPYVPNALANRLARNPVAIANNVYADRMGNGCEQ
DGDGWKYRGRGLIQLTGKSNYSLFAEDSGMDVLEKPELL
ETPAGASMSSAWFFWRNRCIPMAESNNFSMVVKTINGAA
PNDANHGQLRINRYLKTIAAINQGS
(SEQ ID NO: 65)
> PlyPa91
MKGKVIGGSAAAVIALAAAALVKPWEGYSPTPYIDMVGV
ATHCYGDTSRADKAVYTEQECAEKLNSRLGSYLTGISQC
IKVPLREREWAAVLSWTYNVGVGAACRSTLVGRINAGQP
AASWCPELDRWVYAGGKRVQGLVNRRAAERRMCEGRS
(SEQ ID NO: 66)
>PlyPa03
MRTSQRGIDLIKGFEGLRLSAYQDSVGVWTIGYGTTRGV
TRYMTITVEQAERMLSNDLRRFEPELDRLVKAPLNQNQW
DALMSFVYNLGAANLASSTLLKLLNKGDYQGAADQFPRW
VNAGGKRLEGLVKRRAAERVLFLEPLS
(SEQ ID NO: 67)

Nucleotide sequences:

In some cases the nucleotide sequence has been optimized for expression in E. coli.

> PlyKp01 | LOCUS WP_032191494
(SEQ ID NO: 30)
gtggagatgagcaataacggcatcaacatgctgaaaggttttga
agggtgcaggctggccgcttatcaggattctgtaggcgtctgga
cgatcggttatggatggactcaacccgtcaacggcgtgccggtt
ggcaagggcatgaccattacgcaggacactgccgatagcctgtt
gcgtagcggtctggtgcaatatgaaaaaggcgttacggggctcg
ttaaagtcaccatcaatcaaaatcagttcgatgcgctggttgat
tttgcctacaacctgggcgtaaaggcgctggaaggatccacgct
gctgaaaaagctgaatgctggcgattacgccggggctgcggctg
agtttccaaaatggaataaagcaggtggcaaggtgttgccgggg
ctggttaagcgtcgggaagccgagcgtacgttatttctggcctg
a
> PlyKp06 | LOCUS WP_063963664
(SEQ ID NO: 31)
atgcaaaccagcgaaaagggtatttccctgatcaaagagttcga
aggctgcaagcttaacgcctaccaggacagcgtcggtgtatgga
cgattggctatggctggactcagcctgtcgacggcaaaccaatc
cgcgccgggatgacgattaagcaggagacagcagagcgcctgct
gaagaccggactggtcagctacgaaagcgatgtgtcccgcctgg
taaaagttggcctgactcaggggcaattcgatgccctggtatcg
ttcacgtacaacctcggcgcccggtcactgtcgacatctaccct
gctgcgaaaactcaacgcaggtgattacgctggcgctgccgatg
agttcctgcgctggaataaagctggtggcaagatcctgaatggt
ctgacccgtcggcgtgaggcggagcgcgctctgttcctgtcgtg
a
> PlyKp09 | LOCUS WP_042714022
(SEQ ID NO: 32)
atggcgaatcaaccgcaacacaccggcgatgctggcgtcgcatt
aatcaaatcttttgaagggctacggctggagaagtatcgcgatg
ccgtcggcaagtggaccattggctacgggcacctgatcctgccg
aacgagaactttccgcgcccgattaccgaagcggaggctgacgc
gctgctgcgcaaggatttgcagacgagcgagcgcggcgtgcacc
ggctggtgacggtcgatctcgaccaggatcagttcgacgcgctg
gtgtcgtttaccttcaacctcggcgccgggaatttgcagagctc
gacgctgctcaagttgttaaatcaaggcgaatatacgcaggccg
ccgaccagtttctgcgctggaacaaagcgggcggcagagtgctg
cccggcctgacacggcggcgtgaagcggagcgggcgctgttttt
gcaggcgggttag
> PlyKp10 | LOCUS WP_048329977
(SEQ ID NO: 33)
atgcaaaccagtcctgaaggaattgcactgataaaagggtttga
aggctgccggctgaccgcataccccgatccgggaactggtggtg
tgccgtggacaattggctatggctggaccctccccatcgacggt
aagccggtaaggccgggaatgactattgaccaggtaacagcgga
tcgtctgcttaaaaccgggctggtgagctacgagagcgatgtgc
tgaagatcgttaaagtgaagctgaatcagaatcaatttgatgcc
ctggtatcgttcgcctacaacgtcggctcccgcgcattatcaac
ttcaactctgctgaaaaagctcaatgctggcgacatcaaaggcg
ctgctgatgagtttctgcgctggaataaagctggcggcaaagtc
ctgaatgggctgacccgccgacgtgaggcggagcgcgctctgtt
cctgtcgtga
> PlyKp13 | LOCUS WP_019725080
(SEQ ID NO: 34)
atgcaaatcagtaataacggtatcgcgctgattaagcgatttga
gggttgtcggttaaccgcatatcccgacccgggcacaggtggtg
atccctggacgattggctacggctggacgggaaaagtagacggg
aagcctatcaggcccggaatgaagattgacgaagcaacggcgga
tcgtctgctgcgcactggcgtagtgagctttgatcaggcggtaa
gcaagatgctcaaagttaccgttacccagaaccagtacgacgcg
cttgtgtcgctggcctacaacatcggtactcgagcgttatccac
atcaacgctgatgaagaagctgaatgcaggtgatgtgaaaggcg
cggctgatgagttccttcgctggaaccggtcaggcggcaaggta
atggctggcctcactaatcgccgcaaggcagagcgagaagtctt
tttatcgtga
> PlyKp16 | LOCUS WP_068987105
(SEQ ID NO: 35)
atgcaaatcagtaataacggtattgcgctgattaagcgatttga
gggttgcaggttaactgcatatcccgacccgggcaccggcggtg
gtccctggacgattggctacggctggacggggaaagtagacgga
aagcctatcaagcccggaatgaagattgacgacgcaacggcgga
tcgcctgctgcgcactggcgtggtgagctttgaccaggcggtaa
gcaagatgctcaaggtctccgttacccagaaccagtatgacgcg
cttgtgtcgctggcctacaacatcggtacgcgagcgttatctac
atcaacgctgatgaagaagctgaatgcaggtgatgtgaaaggtg
ccgctgacgcattcttgagctggaaccgttcaggcggcaaggta
atggctggcctcaccaatcgtcgcaaggcagagcgggaagtctt
tttatcgtga
> PlyKp17 | LOCUS WP_044067377
(SEQ ID NO: 36)
atgcagataagcgataacggcatcgcactgattaaggggtttga
aggatgtcgattaaccgcatacccggacccgggcaccggcggtg
atccctggacgattggtttcggctggacggggaaagtagacggc
aagcctatcaagccgggaatgaagattgacgatgcgacagcgga
tcgcctgctgcgcactggcgtggtgagctttgacctggcggtaa
gcaagatgctcaaagtttccgtcacccagaatcagtacgacgcg
cttgtgtcgctggcctataacatcggtacgcgagcgctatccac
ctcaacgctgatgaaaaagctgaatgcaggtgatgtgaaaggcg
cagctgatgagttccttcgctggaataaatcaggcgggaaagca
atgtctgggctaaccaatcgccgcaaggcagagcgagaagtatt
tttatcgaaaacacggggaagttatgaactatctcattaa
> PlyKp57 | LOCUS WP_048333081
(SEQ ID NO: 37)
atgaacccgacgctgaggaataagctgattggtgcgatcgccgg
cggttcgggcgcgatagtcattgcttccgtcatgcttggtaatg
Ctgacggcctggaaggaaggcgttattacgcctatcaggatgtt
gtcggcgtctggactgtttgtgatggtcacactggcaccgatat
tcgccgcggccaccgctacaccgacagagaatgcgacaacctgc
tgaaggctgatctgcggaaggtggcaagcgccattgacccgctt
atcaaagtcagcattcctgaccccacccgcgccgcgctttactc
attcacctacaacgttggctctggagctttcgccagttccacgc
tgctgaagaaactgaatgctggagatgtgccgggcgcgtgcaag
gaactgcagcgctggacatacgctggcgggaagcagtggaaagg
ccttatctcaaggcgcgagattgagcgcgaagtttgtctgtggg
ggcagaaatga
> PlyKp61 | LOCUS SAT14280
(SEQ ID NO: 38)
atggtaatgtcaccaaagctcaggaatagcgttcttgctgccgt
tggtggtggtgctattgccattgcgtcggctctcatcaccgggc
caaccggcaatgatggtctggagggagtgaggtataagccgtat
caggatgttgtaggcgtctggacagtctgctatggccacactgg
caaagatatcatgctcggtaaaacctacaccgagtcagagtgtc
gcgcgctgctcaacaaagacctgaacatcgtcgcacgccagatc
aacccgtacatccagaagccgatccccgaaacaatgcgtggggc
tctgtactcgtttgcttataacgtaggcgccggaaacttacaga
cctccactctgctgcgcaaaatcaaccagggcgaccagaaaggt
gcatgcgaccagttgcgccgctggacttatgccaaaggaaagca
gtggaaaggcctggtaactcgccgcgagattgagcgcgaagttt
gtctttgggggcagaaatga
> PlyKp68 | LOCUS WP_048264621
(SEQ ID NO: 39)
atgcgaatcagcagtaatggcgttgtccggctcaaaggcgaaga
aggcgagcgcctcagtgcttatctggatagtcgcggcatcccaa
ccataggcgttggccacacaggaacagtcgacggcaagccagtg
gtgatcggtatggttatcagccagaacaaatcgactgagctgct
gctgcaggatatccagtgggtagagaaggcgatcaacagctcggt
gaaaaccccgcttacgcagaaccagtacgatgcgctgtgcagcct
ggtatttaacatcggggctacagcattctacggttctacggtcct
gaagcgagtgaaccagaaagactacaccgccgctgctgatgcgtt
cctgatgtggaagaaagccggcaaagaccaggaaattctactacc
ccggaggcggcgcgagcgtgcgctgttcctgtcgtga
> PlyKp75 | LOCUS WP_024622713
(SEQ ID NO: 40)
atgaacccgacgctgaggaataagctcattggtgcgattgccggc
ggttcgggtgcgatcgcgattgcttctgtcatgcttggtaatgct
gacggcctggaaggaaggcgttattacgcctatcaggatgttgtc
ggcgtctggactgtttgtgatggtcacactggcaccgatattcgc
cgcggccatcgttataccgacagggaatgcgacagcctgctgaaa
gccgatctgcggaaggtggcaagcgccattgatccgctcatcaaa
gtccgcattcctgatcctacccgcgccgcgctttactcattcacc
tacaacgttggctctggcgctttcgccagctccacgttgttgaag
aaactgaatgctggagatgtgccgggcgcgtgcaaggaactgcag
cgctggacgtatgccggtggcaagcagtggaaggggctgatcacc
aggcgcgagattgagcgtgaagtctgcgagtggggccagaaatga
>PlyKp86 | LOCUS WP_057216474
(SEQ ID NO: 41)
atgaacccgacgctgaggaataagttgattggtgcgatcgccggc
ggttctggtgcgatcgcaattgcttctgtcatgcttggaaatgca
gacggcctggaaggaaggcgttattacgcctatcaggatgttgtc
ggcgtctggactgtttgtgatgggcacactggcaccgatattcgc
cgcggccaccgttacaccgaccgagaatgcgacaacctgctgaag
gcagatctgcggaaggtggcaagcgccattgatccgctcatcaaa
gtccgccttcctgctcctacccgcgccgcgctttactcattcact
tataacgttggctctggtgccttcgccagctccacgctactgaag
aaactgaatgctggagacgtccctggcgcgtgcaaggaactgcag
cgctggatgtatgccggtggcaagcagtggaagggcctgatcacc
aggcgcgagattgagcgtgaagtctgcgagtggggccagaaatga
> PlyEa02 | LOCUS WP_063159646
(SEQ ID NO: 42)
atgcaaaccagcgaaaagggcattgccctgatcaaagagttcgaa
ggctgcaaactcaccgcctaccaggacagcgtcggcgtctggacg
atcggctatggctggactcatcctgtcgacggaaaaccaatccgc
gccgggatgacgattaagcaggaaacggcagaacgcctgctgaaa
actggactggtcagctacgaatgcgacgtgtctcgcctggttaag
gtggggctgactcaagggcagttcgatgctctggtgtcgttcacg
tataacctcggagcccgttcactgtcgacatcgactcttctgcga
aaactcaacgccggtgattacgctggcgcagccgatgagttcctg
cgctggaataaagctggcggtaaagtcctgaatgggctcacccgt
cgtcgggaggcagagcgggctctgttcctgtcatga
> PlyEa04 | LOCUS WP_058675961
(SEQ ID NO: 43)
atgcaaatcagtgatgaaggcattgcgcttattaaaggtttcgaa
gggtgccgattgacagcatatcccgaccctggcaccggtggcgca
ccatggaccataggttacggctggacattgccagttgatggcaag
ccggtacgtccgggtatgacgatcgatcaggctacagctgaccgc
ctgcttaaaatcggtctggtgggctacgaaaacgacgttctgaaa
attgtgaaggtgaagctaacccaagggcagtttgatgccctggtg
tcgtttgcctacaacatcggctcccgcgcactctcaacctccact
ctgctgaagaaacttaatgccggcgatatcaaagacgctgcagat
gagttcctgcgttggaataaagcaggtggcaaggtcctgaatggg
ttgacccgtcggcgtgaggcggagcgcgctctgttcctgtcgtga
> PlyEa06 | LOCUS WP_045381882
(SEQ ID NO: 44)
atgcaagtaagtgataacggtattgtttttttaaagaatgaagaa
ggcgaaaagttaacgggttacccggactcacgcggcattccaaca
atcggcgtgggccacaccggaaaagttaacggtgtgccggtaagt
gtcgggatgaaaataacatcagagcagtcgtcagaactgcttaaa
gatgatttaagctgggttgaagacagcattgcaaattatgttaaa
tcgccactgaatcagaatcagtatgacgcattgtgcagttttatc
ttcaatatcggcgcaccggcgtttgaaggttcaacaatgctcaag
ctgttaaacaagtcggattatgtcggcgcatccggtgaattcccg
aaatggaagcgagccggtaatgacccggatattttgctgccgcga
cgcatgcgcgaacaggctttatttttatcatga
>PlyEa09 | LOCUS WP_059444542
(SEQ ID NO: 45)
atgcaaatcagcagtaacggaatcaccaaactcaaacgcgaagaa
ggcgagaggcttaaggcttacccagatagccgtggaatcccgaca
atcggcgtgggccatacaggcaatgttgatggaaagcctgtaaca
cttggaatgacaatcacatcagataagtcatctgagcttctgaaa
gctgacttgcgatgggtggaagatgcaatcagcagcctggttcgc
gttccactgactcaaaaccagtatgatgcgctttgcagtttgata
ttcaacattggtaaatctgcgtttgcaggctccactgttctgcgc
caactaaaccttaagaattaccaggcagcggctgatgcattcctg
atgtggaagaaagcaggtaaagatactgaaatcctacttccacgg
aggcagagagaaagggctctgttcctgtcatga
> PlyEa10 | LOCUS WP_047076801
(SEQ ID NO: 46)
atgaacccgacgctcaggaataaactgattggcgccatcgccgga
ggttccggcgcgatcgcaattgcctctgtcatgcttggtaacgct
gatgggctggaagggcggcgctattacgcttatcaggatgttgtt
ggcgtctggactgtttgtgatggacataccggttcagatattcgc
cgcggtcaccgctactccgacaaagagtgcgataacctgctgaag
tcagacctgcgaaaggttgctaacgccatcgacccgctgattaag
gttcgcatccctgatcctacccgtgccgctctttactccttcact
tataacgttggctctggtgccttcgccagttccacgctactgaag
aaattgaatgctggagacgtgccgggtgcgtgcaaagaactgcag
cgctggacgtatgccggtggcaagcaatggaagggcctaattacc
cgacgcgagattgagctcgaagtctgtgagtggggccagaaatga
> PlyEa14 | LOCUS WP_042895492
(SEQ ID NO: 47)
atgaatcaacccttgcgaaaatatgtattgtctgcggtcggtggt
ggtgcaattgccatagcctctgcgcttatcactggccctacgggt
aacgatggccttgagggtgtgcgatatcagccttaccaggatgta
gttggcgtctggactgtctgctatggacacactggcaaagacatt
atgctggggaatacttacacgaaatcagagtgtgatgctcttctg
gataaagacctcaacaccgtcgctcgtcagattaacccgtacatc
aaaaagccaatccctgaaacgatgcgtggggcgctgtactcattt
gcctataacgttggtgctggcagctttcagacttcaacgctgctg
cgcaaaattaaccagggggattcgaaaggtgcctgtgagcagtta
cgcgtctggatttacgcggggaaaaaggtctggaagggattggta
actcgccgtgaaattgagcgcgaggtgtgtttgtggggccaaaaa
tga
> PlyEa16 | LOCUS WP_063447397
(SEQ ID NO: 48)
atgaatccttcaatcgttaagcgctgccttgtcggggcggtgct
ggctattgctgccacgctgcccggtttccagtcgcttcatacct
ccgttgaggggctgaaactgattgccgattacgaggggtgccgc
ctgcagccttatcagtgcagcgcgggcgtgtggaccgacgggat
cggcaatacgtccggtgtggtgccgggcaaaaccatcacggaac
ggcaggcggcgcagggacttatcactaacgtactgcgcgtggag
cgggcgctggataaatgtgtggcgcagccgatgccgcaaaaagt
ctatgacgcggtggtgtcgtttgctttcaacgtgggcaccggca
acgcctgcagctccacgctggttaagttgctgaaccagcggcgc
tgggcagatgcctgccatcagctgccgcgctgggtatatgtcaa
aggtgtgtttaatcaggggctggacaatcgccgcgcgcgggaaa
tggcctggtgcttaaaaggagcataa
> PlyEa36 | LOCUS KZQ44728
(SEQ ID NO: 49)
atggcaatgtcaccggcgctcagaaatagcattgttgcagccct
cggtaccggtgctattggtatcgcgaccgtcatggtttctggaa
agtcaggcctggagggtagagagcattacccatacaaagatatt
gttggcattgtcaccgtttgtgatgggtatacaggaagcgatat
tgtctggggtaaatattactcagacaaagaatgtgatgcgttga
cgcgtaaagatatgacgcgaattgctgcacaagttaatccgcat
atcaaagtgccgaccactgaaacacagcgagctgcaatatatag
cttcgcttacaacgtcggatccacagcagccatcaactcaaccc
tgttgaagaaactcaactctaaagattactccggggcatgctca
gagcttaagagatgggtatatgcaggtggaaagaaatggaaagg
cctgatgaaccgacgcgacgttgagtacgaggtttgcacctgga
gccagaaatga
> PlyEa41 | LOCUS WP_063411204
(SEQ ID NO: 50)
gtgagctcaatcgttaaacgttgcagtgtggccgcagtgctggc
actggcggcattgttgcctgactttcgtctgctgcatacctcgc
ctgatggtctggcattgattgctgaccttgaagggtgccgcctg
gcaccttaccagtgcagtgcgggcgtgtggacgtcaggcatcgg
ccacactgccggggtggtaccaaaacgcgatatcaccgagcgcg
aagcggcggcaaatctggtcgccgacgtgctgaataccgagcgc
cgtctcgcggtctgcgtgccggtcaccatgccgcagcctgttta
cgacgcgctggtcagtttctcttttaacgtcggcaccggcgcgg
cttgtcgctcgacgctggtctcttacatcaagcgtcatcagtgg
tggcaggcatgcgaccaacttagccgctgggtgtacgtcaacgg
ggagcgtagcaccggacttgaaaatcgacgtcagcgagagcgtg
cttattgcctgaagggggtgaaatga
> PlyEa42 | LOCUS WP_049056838
(SEQ ID NO: 51)
atgacgaacaaagtaaagtttagcgctgccatgctggcgcttct
cgctgccggagcaacagcaccagaattgtttgaccagttcatga
gtgagaaagaaggtaatgcgctggtggctgtcgttgatcctggc
ggcgtctggtcgttatgtcatggcgttatctttatcgatggcaa
gcgtgtcgtaaaaggtatgacggcgactgagtctcaatgtcgaa
aagtgaatgcaatcgagcgtgataaggcgctgtcgtgggttgac
cgcaatatcaatgttcccctgaccgagccgcaaaaagtcggtat
tgcgtcattctgcccatacaacatcggcccaggtaaatgctttc
cttcgacgttttataagcgcatcaatgcaggtgaccgtaaaggg
gcatgcgaagcaatccgctggtggattaaggacggtgggaagga
ttgccgcatacgctctaataactgctacgggcaggtaactcgcc
gggatcaggaaagtgcgctgacgtgctgggggattgaccagtga
> PlyEa43 | LOCUS WP_058650108
(SEQ ID NO: 52)
atgagcaacaaagctaaattcagcgccgctatgctggtgcttct
ggccgccggtgcgtcagcgccggtgctgttcgatcagtttattg
gtgaacgcgagggtaactcgctaacggcggttatcgatcccggt
ggggtttggtcaatatgccggggggtaacacgcatcgatggccg
cccggtagtgaaggggatgaacttaacgcagagccagtgtgacc
attacaacgcaatcgaacgcgacaaggcgctggcgtgggtacaa
aagaatgttcacgttccactaactgagccgcagaaagtcggcat
tgccagcttttgcccgtacaacatcgggccggggaagtgttttc
cttcgacgttttatcgcaagctaaatgccggcgaccgcaaaggg
gcatgcgcggagatccggcgctggatattcgacggcggcaggga
ttgccggttaacgaaagggcaggccaacggctgttacgggcagg
ttgaccgacgcgatcaggaaagtgcgctgacgtgctgggggctt
tacgaatga
> PlyEa62 | LOCUS WP_059304620
(SEQ ID NO: 53)
atggcatccctgaaaacgaaactcagcgcagccatgctgggatt
aatagctgctggtgcatccgccccaaccttgatggatcagttcc
tggatgagaaagaaggtaacagccttaccgcttatcgcgatggt
agccaggggatctggactatttgcagaggcgccacgcgaattga
tggtaaacccgtcacgcagggaatgaagttgacccaggccaaat
gcgacgaggtgaatgatatcgaacgtgataaggcactggcgtgg
gttgatcggaatatccgcgtaccgttgacgcctccgcagaaagt
cggcattgcttcattctgtccgtacaacatcggccccggtaaat
gcttcccgtctacgttctaccagcgcatcaacgccggcgaccgt
aaaggcgcatgtgaagcgattcgctggtggattaaggacggtgg
gaaggattgccgcatacgctctaataactgctacgggcaggtaa
ctcgccgggatcaggaaagtgcgctgacgtgctgggggattgac
cagtga
>PlyKp104
(SEQ ID NO: 54)
atggcatggggtgccaaggttagtaaagagttcaagttaaaggt
gattgaggtgtgcgaacgccttgaaattaaccctgactacttga
tgagctgcatggcttttgaaacgggcgagacgttctcaccaaat
gtccgcaatccgaatgggtccgccactggcttgatccagtttat
gtccaacacagctcgcagtctgggtactacgacaaatgagttag
cagacatgacctctgttgagcaaatggactacgtggagaagtac
tttaagccgtatgctgggaaaatcaagacgattgaggatgtata
catggtgattttttgccctcgtgccgttggaaaacctgactcgt
atattctttacgacgaaggtcgtagttacaacgacaataaaggg
ttggaccttaataaggacaatgctattactaaatacgaggctgg
attcaaggtgcgtgagaaactgaagttaggtatgaaagagggtt
accgtggttaa
>PlyKp105 (internal name KLB-493-1)
(SEQ ID NO: 55)
atggctaacctgaaaacgaaactcagttcggccatgctggcgct
tatcgctgctggcgcttcagctcccgttcttatggaccagttcc
tgaatgagaaagagggcaaaagcctcacgtcataccgcgatggc
gccggcatatggacgatatgtcgtggagttacccaggtagatgg
aagacctgtaacccagggaatgaagttaacccaggccaaatgcg
atcaggttaatgccgtcgagcgcaataaggcgctggcatgggta
gatcagaatgtgcgtgttcctctgacaccccctcaaaaggtcgg
gattgccagtttctgcccctataacatcgggcccggtaaatgct
ttccttccaccttctaccgcaagctgaatgccggtgaccggaaa
ggcgcctgcgctgaaattcgccggtggatttttgatggcggaaa
agattgccgcgtgcgttcgaacaattgttacggccaggtctctc
gtcgtgatcaggaaagcgcactggcatgttgggggatagatgaa
>PlyKp17/LALF (lysin + antimicrobial peptide)
(SEQ ID NO: 56)
atgcagataagcgataacggcatcgcactgattaaggggtttga
aggatgtcgattaaccgcatacccggacccgggcaccggcggtg
atccctggacgattggtttcggctggacggggaaagtagacggc
aagcctatcaagccgggaatgaagattgacgatgcgacagcgga
tcgcctgctgcgcactggcgtggtgagctttgacctggcggtaa
gcaagatgctcaaagtttccgtcacccagaatcagtacgacgcg
cttgtgtcgctggcctataacatcggtacgcgagcgctatccac
ctcaacgctgatgaaaaagctgaatgcaggtgatgtgaaaggcg
cagctgatgagttccttcgctggaataaatcaggcgggaaagca
atgtctgggctaaccaatcgccgcaaggcagagcgagaagtatt
tttatcgaaaacacggggaagttatgaactatctcatggtaccg
gaggtggatcaggtggaggttctggaggaggtgaccatgagtgt
cactatcgtatcaaaccgacatttcgccgtctgaaatggaagta
taaaggtaaattttggtgccccagttaa
>PlyKp17/LL-37 (lysin + antimicrobial peptide)
(SEQ ID NO: 57)
atgcagataagcgataacggcatcgcactgattaaggggtttga
aggatgtcgattaaccgcatacccggacccgggcaccggcggtg
atccctggacgattggtttcggctggacggggaaagtagacggc
aagcctatcaagccgggaatgaagattgacgatgcgacagcgga
tcgcctgctgcgcactggcgtggtgagctttgacctggcggtaa
gcaagatgctcaaagtttccgtcacccagaatcagtacgacgcg
cttgtgtcgctggcctataacatcggtacgcgagcgctatccac
ctcaacgctgatgaaaaagctgaatgcaggtgatgtgaaaggcg
cagctgatgagttccttcgctggaataaatcaggcgggaaagca
atgtctgggctaaccaatcgccgcaaggcagagcgagaagtatt
tttatcgaaaacacggggaagttatgaactatctcatggtaccg
gaggtggatcaggtggaggttctggaggaggtttgcttggagac
ttttttcgcaaatccaaggagaaaattggcaaggaattcaagcg
tattgtacagcgcatcaaggactttctgcgcaacttggtcccgc
gtacagaaagt
>PlyKp117/RI-18 (lysin + antimicrobial peptide)
(SEQ ID NO: 58)
atgcagataagcgataacggcatcgcactgattaaggggtttga
aggatgtcgattaaccgcatacccggacccgggcaccggcggtg
atccctggacgattggtttcggctggacggggaaagtagacggc
aagcctatcaagccgggaatgaagattgacgatgcgacagcgga
tcgcctgctgcgcactggcgtggtgagctttgacctggcggtaa
gcaagatgctcaaagtttccgtcacccagaatcagtacgacgcg
cttgtgtcgctggcctataacatcggtacgcgagcgctatccac
ctcaacgctgatgaaaaagctgaatgcaggtgatgtgaaaggcg
cagctgatgagttccttcgctggaataaatcaggcgggaaagca
atgtctgggctaaccaatcgccgcaaggcagagcgagaagtatt
tttatcgaaaacacggggaagttatgaactatctcatggtaccg
gaggtggatcaggtggaggttctggaggaggtcgcaagaagact
cgtaagcgcctgaagaaaatcgggaaggtgttaaaatggatt

### Example 10. Transglycosylase Lysins to Kill Klebsiella Pneumoniae and Pseudomonas Aeruginosa

The bacterial cell wall peptidoglycan (PG) is composed of glycan chains of alternating N-acetylglucosamine (GlcNAc) and N-acetylmuramic acid (MurNAc) that are cross-linked through peptides connected to the lactyl moiety of MurNAc. This heteropolymer produces a mesh-like sacculus that surrounds the bacterial cell imparting strength, support, and shape, as well as resistance to internal cytoplasmic pressures. Maintaining the integrity of the PG sacculus is vital to cell viability and its importance is reflected by the number of different classes of antibiotics that target PG biosynthesis, including the glycopeptide vancomycin and the β-lactams. However, the PG sacculus is not a static structure, but is continually expanding and turning over. A class of enzymes responsible for cleaving PG to accommodate these requirements are termed lytic transglycosylases (LT). This class of lytic enzymes lyse the PG with the same substrate specificity as lysozymes, i.e., the β-1,4 glycosydic bond between MurNAc and GlcNAc. However, unlike lysozymes, the LTs are not hydrolases but instead cleave PG with concomitant formation of an intramolecular 1,6-anhydromuramoyl reaction product.

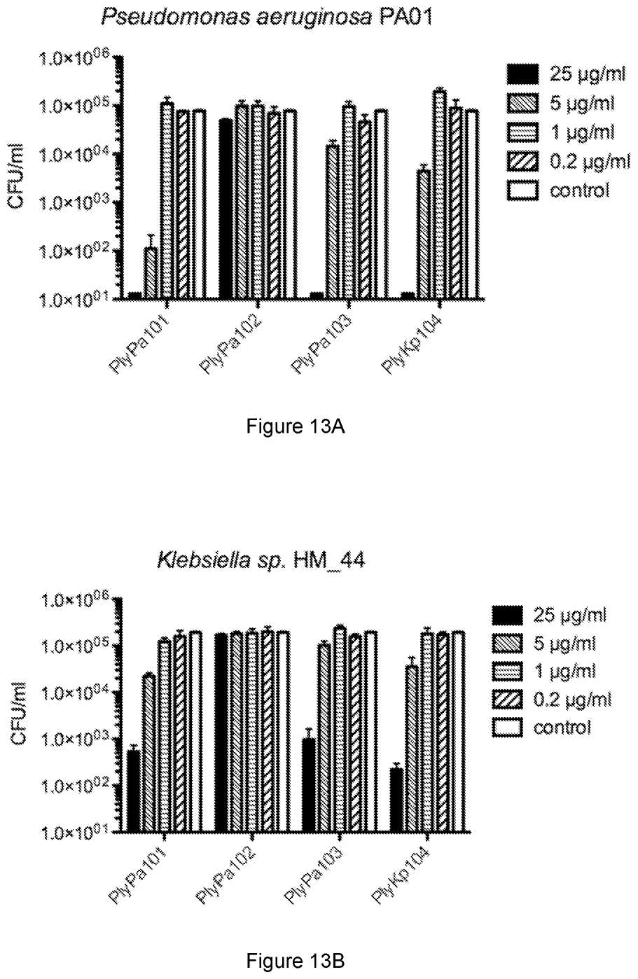

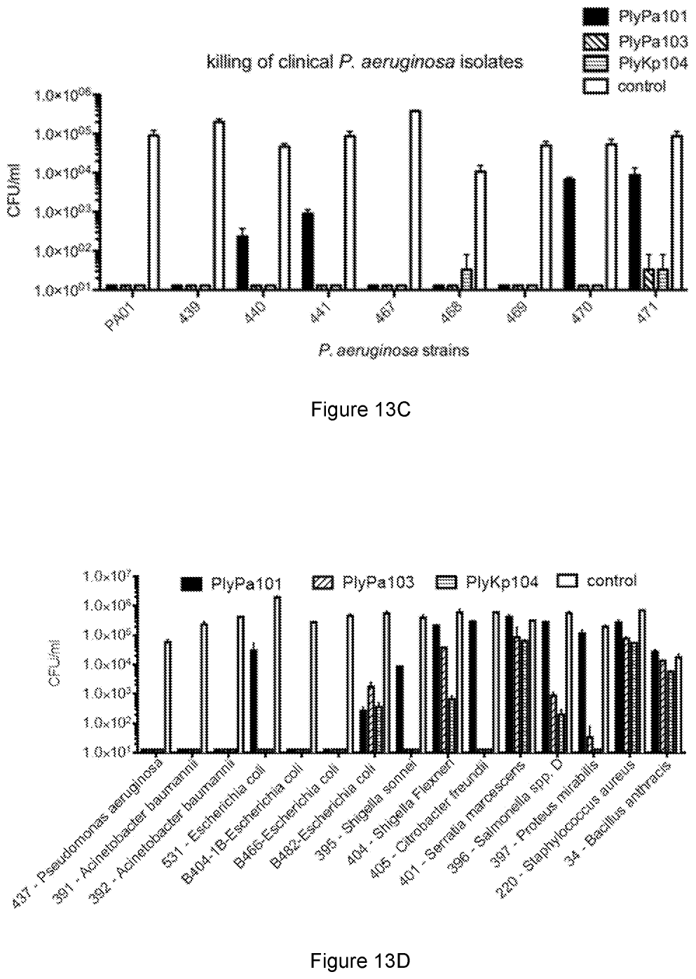

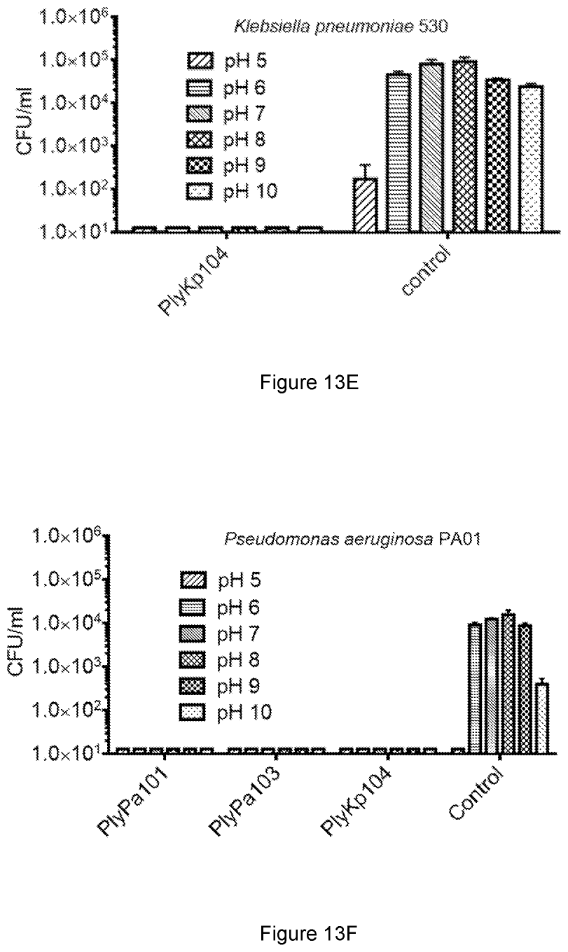

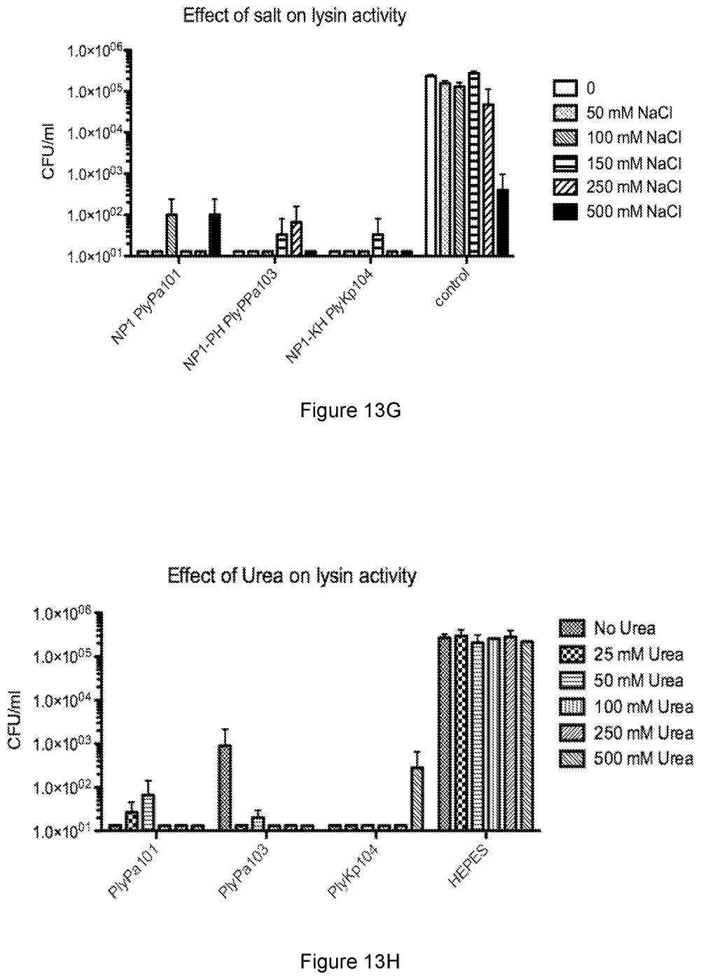

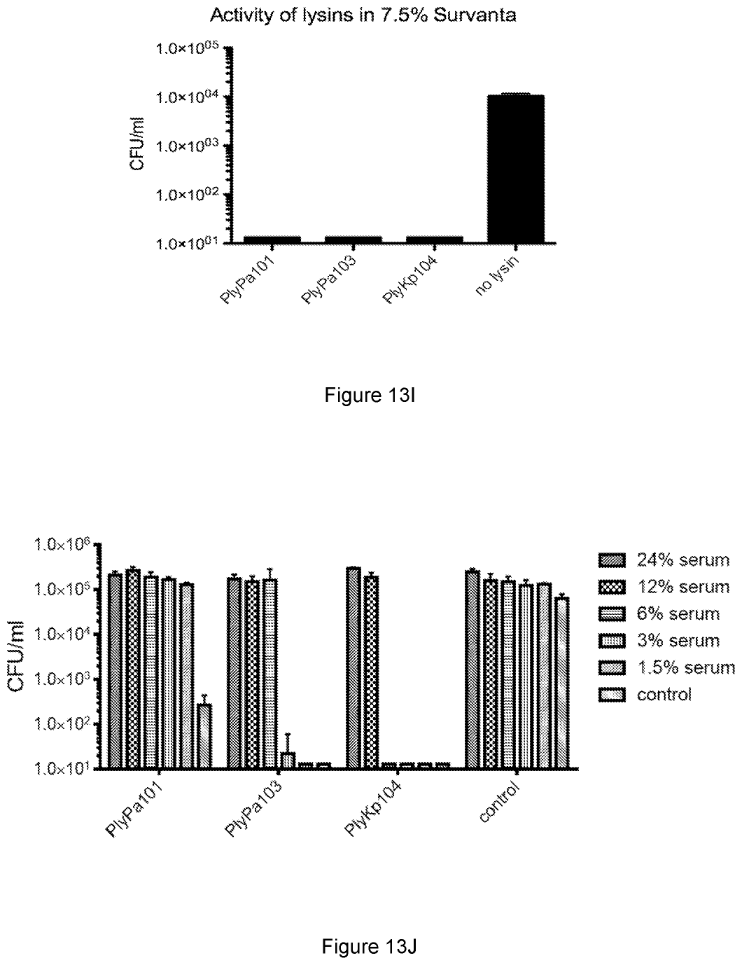

Phage lysins PlyKp104 is a Klebsiella pneumoniae enzyme and PlyPa101, PlyPa102, and PlyPa103 are Pseudomonas aeruginosa lytic transglycosylase enzymes. This class of lytic enzymes were tested for their activity on Klebsiella and Pseudomonas strains. FIG. 13 demonstrates the bactericidal activity of transglycosylase lysins against P. aeruginosa PA01 (FIG. 13A) and Klebsiella sp. HM_44 (FIG. 13B). At 25 μg/m1 PlyPa101, PlyPa103 and the Klebsiella enzyme PlyKp104 were active against Pseudomonas strain PA01 showing >5-log kill. The same three enzymes exhibited a 2-log kill of the Klebsiella strain HM_44. The remainder of FIG. 13C-J is as explained in the description of the figures above.

### Example 12 Lysins to Control Topical and Mucosal Bacterial Infections

### Methods

Ethics statement

Samples from human subjects were obtained in accordance with protocol VFI-0790, approved by Rockefeller University Institutional Review Board, and all subjects gave an informed consent. Mouse work was performed in accordance with protocol number 14691H, approved by the Rockefeller University's Institutional Animal Care and Use Committee. All experiments were conducted at The Rockefeller University's animal housing facility, an AAALAC-accredited research facility, with all efforts made to minimize suffering.

### Bacterial Strains and Growth Conditions

Table S1 describes bacterial strains used in this study and their source. Gram-negative bacteria were cultured in lysogeny broth (LB, EMD Millipore), and Gram-positive bacteria were grown in Mueller Hinton broth (Difco) at 37° C., with shaking at 200 rpm.

### Gene Synthesis and Cloning

To facilitate preliminary screening of Pseudomonas lysins, pAR553, a derivate of pBAD24 containing a new MCS (EcoRI—SalI—NotI—KpnI—XbaI—PstI), was constructed by aligning primers 629_5_pBAD_MCS (5′-attcgtcgacggggcggccgcggtacctctagactgcag) (SEQ ID NO:61), and 630_3_pBAD_MCS (5′-gtctagaggtaccgcggccgccccgtcgacg) (SEQ ID NO:62), and inserting the resulting double-stranded DNA into the EcoRI and PstI sites of pBAD24. Pseudomonas lysins were identified in the NCBI database through BLAST search using the Acinetobacter lysin PlyF307 as query, yielding over 100 hits. All hits were aligned using the Lasergene MegAlign Pro software, with the MUSCLE algorithm. A candidate was selected from each group (see Table S2 for protein identifiers). Nucleotide sequences for selected lysins were designed with an upstream SalI and a downstream NotI restriction sites, and were synthesized by Genewiz. Creation of plasmids for the initial screen was done by inserting the lysin sequence into the SalI and NotI sites of pAR553. Creation of a 3C-cleavable hexahistidine-tagged versions of the lysins was done by inserting the lysin sequence into the SalI and NotI sites of a modified pET21 vector.

### Purification of Phage Lysins

An overnight culture of E. coli BL21 containing a lysin cloned into a modified pET21a vector was diluted 1:100 into 1 L of LB medium containing ampicillin, and placed in an environmental shaker. Upon reaching OD₆₀₀ 0.5, the expression of the lysin was induced with 0.2 mM IPTG for 4 h at 37° C., and the cells were then shaken overnight at 4° C. The cells were harvested and resuspended in 40 ml MCAC buffer (30 mM Tris pH 7.4, 0.5 M NaCl, 10% glycerol, 1 mM DTT), and homogenized using an Emulsiflex-C5 homogenizer (Avestin, Ottawa, Ontario, Canada). Cell debris was removed by centrifugation, and the supernatant was filtered through a 0.22-μm filter (Millipore). The cleared lysate was loaded on a NiNTA column equilibrated with MCAC buffer, followed by washes with MCAC containing 20 mM imidazole and elution with MCAC containing 150 mM imidazole. The eluted fraction was supplemented with 10×3C buffer for a final concentration 150 mM NaCl, 50 mM tris pH 7.6, 10 mM EDTA, 1 mM DTT, and 50 μl of 3C protease were added per 1 mg of purified protein. The mix was incubated overnight at 4° C., placed in a dialysis bag with a 3 kDa cutoff, and dialyzed for 24 h against PBS with 3 buffer changes. The protein was then concentrated using an Amicon ultrafiltration device, fitted with a 3-kDa molecular weight cutoff membrane, and the final concentration was determined using a ND-1000 spectrophotometer (Nanodrop), according to absorbance at 280 nm.

### Overlay Assays

To prepare P. aeruginosa overlay agarose, strain PA01 was grown overnight in 6 L of LB medium, harvested, and suspended in 3 L PBS. The cells were aliquoted into bottles containing agarose to a final concentration of 0.7%, autoclaved, and stored at 4° C. until use.

E. coli strains containing a lysin gene in pAR553 (pBAD24-based) were streaked on LB+ampicillin 15 cm glass plates containing 0.2% arabinose (to induce protein expression) overnight at 37° C. The plates were exposed to chloroform vapor for 5 minutes to permeabilize the cells. Then, soft agar containing autoclaved (to destabilize the outer membrane) P. aeruginosa cells at 50° C. was poured over the plates, covering the cells. The plates were incubated at 37° C. and examined for the presence of clearing zones following 1, 2, 5, and 16 hours.

To test activity of the lysins in crude lysate, E. coli strains containing the gene in pAR553 were diluted 1:100 from an overnight culture into 400 ml LB+ampicillin and grown at 37° C. with shaking at 200 RPM. Once the cultures reached OD₆₀₀ 0.5, arabinose was added to a final concentration of 0.2% to induce expression of the lysin. The cells were incubated for 4 h at 37° C., and placed at 4° C. with gentle agitation overnight. Cells were harvested, suspended in 40 ml PBS, and homogenized. Cell debris was removed by centrifugation, and the supernatant was filtered through a 0.22-μm filter (Millipore). Varying amounts of the cleared lysate was applied to a 15 cm plate containing autoclaved P. aeruginosa agarose. Observations for the presence of clearing zones were done following 1, 2, 5, and 16 hours.

### Bactericidal Assays

An overnight culture of the test bacteria was diluted 1:50 into fresh LB medium and grown to OD₆₀₀ 0.5. The cells were harvested, washed, and suspended in 30 mM HEPES buffer pH 7.4 to a final concentration of about 10⁶ cells/ml (unless otherwise noted). In a U-bottomed 96-well plate, each lysin was diluted to the desired final concentration in 50 μl 30 mM HEPES buffer, and then 50 μl of the test bacteria were added to each well. The plate was incubated for 1 h at 37° C. with shaking at 200 RPM. The content of each well was then serially diluted 10-fold and streaked on LB plates to quantify viable bacteria. Mueller Hinton agar plates were used in experiments with Gram-positive bacteria.

In time kill curves, following incubation, assay contents were diluted 1:1 in 5% BBL Beef Extract (BD) to stop the reaction, and were immediately diluted and plated. Assays evaluating the effect of pH were done by adding 25 μl of 100 mM of the following buffers to wells of a 96-well plate (final concentration 25 mM): pH 5.0—acetate buffer; pH 6.0—MES buffer; pH 7.0 and 8.0—HEPES buffer; pH 9.0—CHES buffer; pH 10.0—CAPS buffer. Bacteria and lysins were diluted in deionized water rather than buffer as not to affect the final pH of the reactions. Assays evaluating the effect of salt, and urea were carried out in 30 mM HEPES Buffer pH 7.4, 100 μg/ml lysins. Evaluation of the effect of serum was done in 30 mM HEPES Buffer pH 7.4, 100 μg/ml lysins, using serially diluted pooled human serum from male subjects, AB blood type (Sigma). Experiments in Survanta (beractant, Abbvie) were carried out in 30 mM HEPES Buffer pH 7.4, and 100 μg/ml of lysins. Kill assays were done in duplicates or triplicates, and repeated at least twice.

### Biofilm Assays

An overnight culture of P. aeruginosa PA01 was diluted 1:1000 in TSB containing 0.2% glucose. The diluted bacteria were added to an MBEC Biofilm Inoculator 96-well plate (Innovotech #9111) at 100 μl/well and placed in a plastic bag with a wet paper towel to maintain humidity. Biofilm was grown at 37° C. for 24 h at 65 RPM. The 96-peg lid, which contained established biofilm, was removed and washed twice using 96-well plates with 200 μl/well PBS. The washed biofilm was then transferred to a 96-well plate containing 200 μl/well of the lysins or controls and placed in a 37° C. shaker at 65 RPM for 2 h. The biofilms were then washed with PBS as described above and transferred to a 96-well plate containing 200 μl/well PBS for recovery by water bath sonication for 30 min. Quantification of surviving cells was done by serial dilutions and plating.

### Cytotoxicity Assay

To evaluate the cytotoxicity of PlyPa03 and PlyPa91, hemolysis of human Red Blood Cells (RBC), was measured. Human blood was obtained from healthy volunteers at the Rockefeller University Hospital, and collected in a conical tube containing EDTA. RBC were harvested by a low speed centrifugation, 800×g for 10 minutes. Cells were washed three times with PBS, and resuspended in 10% of the volume of PBS. In a 96-well microtiter plate, 100 μl of the human RBC suspension was mixed 1:1 with PlyPa03 or PlyPa91 to yield final concentrations ranging from 1 to 200 μg/ml. PBS and 1% Triton X-100 were used as negative and positive controls, respectively. The 96-well microtiter plate was then incubated for 4 hours at 37° C., 5% CO₂. The intact RBCs were sedimented by low speed centrifugation and 100 μl of the supernatant was transferred into a new microtiter plate. The absorbance was measured at OD₄₀₅ₙₘ using a SpectraMax M5 (Molecular Devices) microplate reader to quantify release of hemoglobin.

### Mouse Skin Infection Model

The skin infection model was based on Pastagia et al. Female CD1 mice, 6-8 weeks old (Charles River Laboratories, Wilmington, Mass.), were anesthetized by an IP injection of ketamine (1.2 mg/animal) and xylazine (0.25 mg/animal). The back of the mice was shaven with an electric razor and treated with depilatory cream to remove the remaining hair. Then, an area of 2-cm2 was tape-stripped 15-20 times using autoclave tape (using a fresh piece of tape each time); two experimental areas were prepared for each mouse, and these were treated in a similar manner (treatment or control) to prevent cross contamination. The tape stripped areas were then sanitized using alcohol wipes, allowed to dry for a few minutes, and then inoculated with 10 μl of log-phase P. aeruginosa PA01 at a concentration of 5×10⁶/ml. Infection was allowed to establish for 20 hours, and the mice were then treated with two sequential 25 μl doses of lysin in CAPS buffered saline pH 6.0 or buffer control. Three hours following treatment, mice were euthanized, and the wound area was excised. Each skin sample was homogenized in 500 μl PBS using a stomacher 80 Biomaster machine (Seward Ltd., United Kingdom). The homogenate was serially diluted and plated on LB plates supplemented with 100 μg/ml ampicillin as a selective agent to prevent growth of normal skin flora (P. aeruginosa is resistant to ampicillin), in order to calculate the P. aeruginosa CFUs in the skin sample.

### Mouse Lung Infection Model

Female C57BL/6 mice, 9-10 weeks old (Charles River Laboratories, Wilmington, Mass.), were anesthetized using isoflurane. Lung infection was established by intranasal instillation of 2×50 μl of 10⁸ CFU/ml log-phase P. aeruginosa PA01. To determine the bacterial load in the lungs before treatment, 2 animals were euthanized 3 h after challenge and the lungs were divided into the top half and bottom half, homogenized in 500 μl PBS, and CFU counts determined. The mean count in both the upper and lower halves was around 10⁶ CFU/ml. The mice were treated at three and six hours post infection with 50 μl of 1.8 mg/ml PlyPa91 or PBS by two intranasal instillations, or by one intranasal and one intratracheal instillation. All treatments were performed on isoflurane anesthetized mice. A 100 μl pipette and tips were used for intranasal application. Intratracheal instillation was performed using a known technique. A 22-gauge catheter was inserted into the mouse trachea using a mouse fiberoptic endotracheal intubation kit from Kent Scientific Corp. Then 50 μl of treatment liquid was added into the bottom of the catheter and injected into the lungs with 200 μl of air from an attached 1 cc syringe. Survival of the mice was monitored daily for 10 days.

### Statistical Analysis

Two-tailed student's t-test was used to evaluate statistical significance in bactericidal assays, biofilm assays, and murine skin models. Data from the murine lung infection model were statistically analyzed using Kaplan-Meier survival curves with standard errors, 95% confidence intervals, and significance levels (log rank/Mantel-Cox test) calculated using the Prism 7 computer program (GraphPad Software, La Jolla, Calif.).

### Results Obtained Using the Foregoing Materials and Methods

Identification of P. aeruginosa phage lysins based on homology search

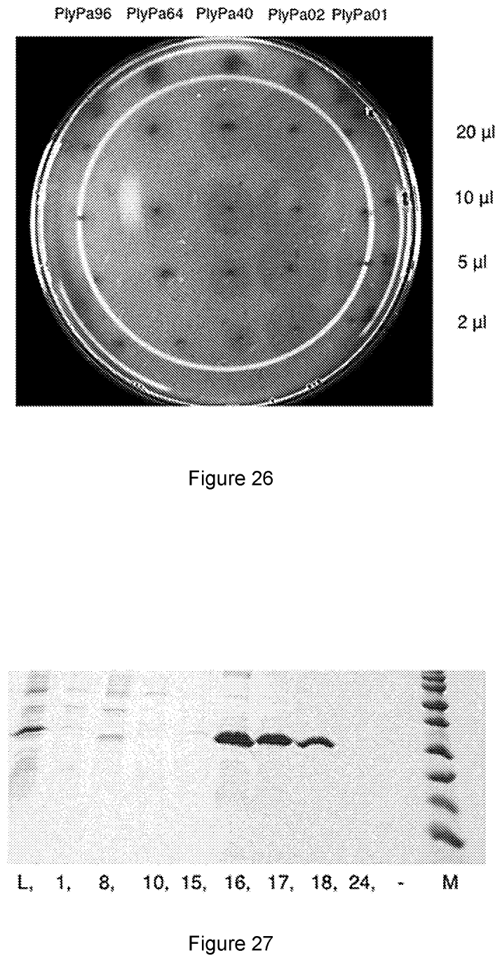

To identify phage lysins with bacteriolytic activity against P. aeruginosa we searched for genes with homology to the Acinetobacter baumanii phage lysin PlyF307 within P. aeruginosa genomes available in the NCBI database, resulting in over 100 hits. We then selected 11 lysin sequences representing all major groups and produced synthetic DNA for each lysin for subsequent protein expression. To screen for catalytically active lysins, these 11 candidates (PlyPa01, PlyPa02, PlyPa40, PlyPa49, PlyPa58, PlyPa64, PlyPa78, PlyPa80, PlyPa91, PlyPa92, PlyPa96) were inserted into pAR533, a pBAD24-based plasmid with an altered multi-cloning site. In one approach, strains containing the expression plasmid were grown on plates containing arabinose to promote expression of the protein. Lysins were released from the streaked cells by exposure to chloroform vapor, and catalytic activity was evaluated by overlaying the plate with soft agar containing autoclaved (to disrupt the outer membrane) P. aeruginosa, and examining the formation of clearing zones around the streaked cells (FIG. 25). In a different approach, an induced lysate of the different strains was applied to a plate containing soft agar with autoclaved P. aeruginosa, and the degree of lysis was evaluated (for a representative image see FIG. 26). A summary of the results obtained is presented in Table 3. The results of the two methods were consistent, with one exception (activity for PlyPa58 was only observed using the crude lysate method). Lysins demonstrating peptidoglycan hydrolase activity in both screening assays (PlyPa01, PlyPa02, PlyPa40, PlyPa49, PlyPa64, PlyPa91, PlyPa96) were characterized further.

TABLE S1
Strains used in this example

| Organism | Source |
|---|---|
| A. baumannii, ATCC 17978 | ATCC |
| A. baumannii, ATCC BAA-1791 | ATCC |
| B. anthracis, Δ Stem | (22) |
| C. freundii, ATCC 8090 | ATCC |
| E. aerogenes, NR-48555 (CRE) | BEI |
| E. Cloacae, NR-50391 | BEI |
| E. Cloacae, NR-50392 | BEI |
| E. Cloacae, NR-50393 | BEI |
| E. coli, DH5α | Invitrogen |
| E. coli, AR531 | NYU Hospital (UTI) |
| K. pneumoniae, ATCC700603 | ATCC |
| K. pneumoniae, ATCC10031 | ATCC |
| K. pneumoniae, ATCC700603 | ATCC |
| K. pneumoniae, NR-15410 (bla_{KPC}) | BEI |
| K. pneumoniae, NR-15411 (bla_{KPC}) | BEI |
| K. pneumoniae, NR-41923 | BEI (Urine) |
| K. pneumoniae, NR-44349 | BEI (Sepsis) |
| P. aeruginosa, PA01 | ATCC |
| P. aeruginosa, AR443 | Cornell Hospital |
| P. aeruginosa, AR444 | Cornell Hospital |
| P. aeruginosa, AR461 | NYU Hospital (LRT) |
| P. aeruginosa, AR463 | NYU Hospital (LRT) |
| P. aeruginosa, AR465 | NYU Hospital (LRT) |
| P. aeruginosa, AR468 | NYU Hospital (wound) |
| P. aeruginosa, AR469 | NYU Hospital (wound) |
| P. aeruginosa, AR470 | NYU Hospital (stool) |
| P. aeruginosa, AR471 | NYU Hospital (UTI) |
| P. aeruginosa, AR472 | NYU Hospital (UTI) |
| P. aeruginosa, AR474 | NYU Hospital (UTI) |
| P. mirabilis, AR397 | Hunter College Collection |
| Salmonella spp. Serogroup D AR396 | Hunter College Collection |
| S. marcescens, AR401 | Hunter College Collection |
| S. flexneri, ATCC 12022 | ATCC |
| S. sonnei, ATCC 25931 | ATCC |
| S. aureus, Newman | (56) |

TABLE S2

| Lysin | Protein identifier |
|---|---|
| PlyPa01 | WP_058157505 |
| PlyPa02 | WP_073667504 |
| PlyPa03 | WP_070344501 |
| PlyPa09 | WP_042930029 |
| PlyPa19 | WP_034013816 |
| PlyPa21 | WP_042853300 |
| PlyPa29 | WP_058158945 |
| PlyPa40 | WP_058171189 |
| PlyPa49 | WP_058355500 |
| PlyPa58 | WP_058182687 |
| PlyPa64 | WP_033973815 |
| PlyPa78 | WP_034067975 |
| PlyPa80 | WP_057386760 |
| PlyPa91 | CRR10611 |
| PlyPa92 | WP_052160556 |
| PlyPa96 | WP_019681133 |

TABLE 3

| Lysin | Colony overlay | Induced lysate |
|---|---|---|
| PlyPa01 | + | + |
| PlyPa02 | + | + |
| PlyPa40 | + | + |
| PlyPa49 | + | + |
| PlyPa58 | − | + |
| PlyPa64 | + | + |
| PlyPa78 | − | − |
| PlyPa80 | − | − |
| PlyPa91 | + | + |
| PlyPa92 | − | − |
| PlyPa96 | + | + |

### Evaluation of Lysin Killing Activity Against P. Aeruginosa And Other Gram-Negative Organisms

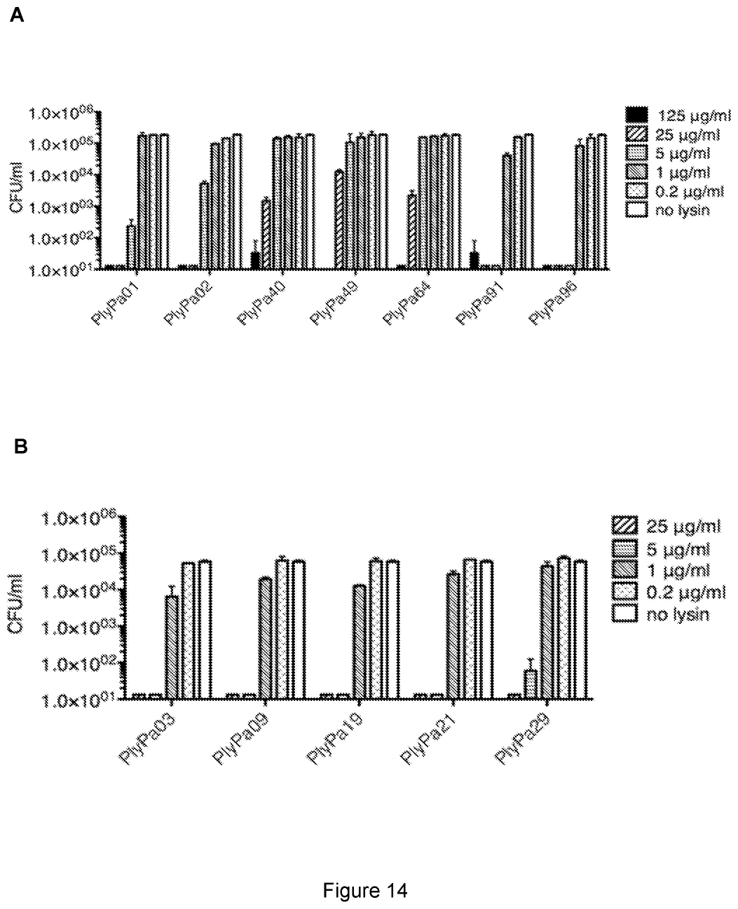

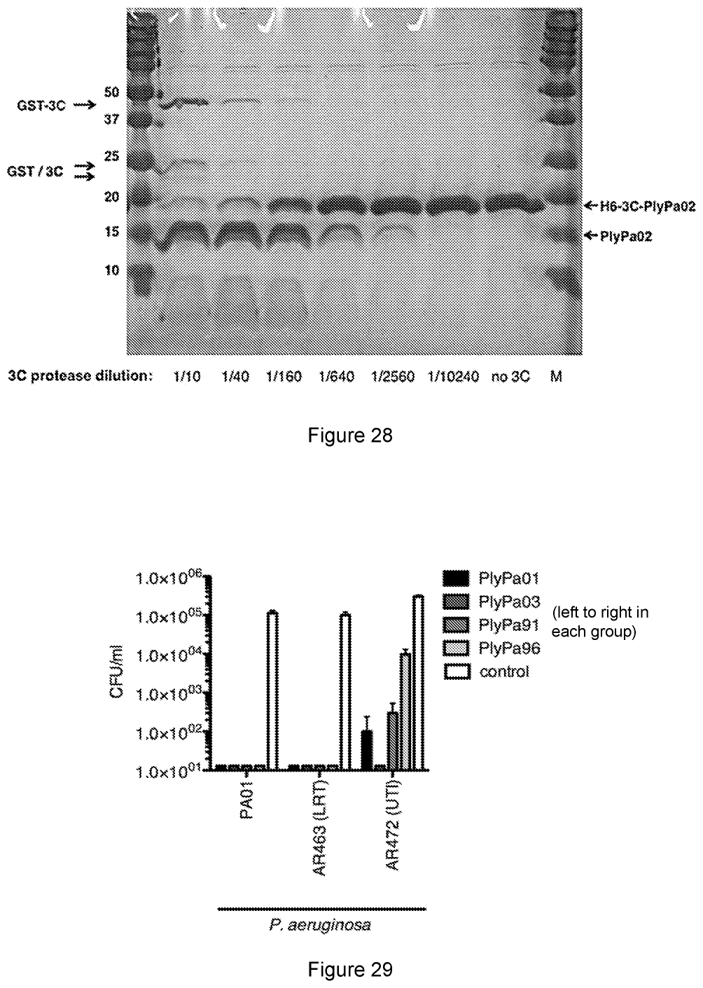

To evaluate the killing activity of the lysins against live P. aeruginosa, we produced 3C-cleavable hexahistidine tag fusion proteins for those lysins that demonstrated catalytic activity against autoclaved Pseudomonas. These lysins were purified by metal ion affinity chromatography (A representative purification is presented in FIG. 27), and the hexahistidine tag was cleaved by 3C protease (an example is presented in FIG. 28). In this manner, the final purified and cleaved product contained only 4 additional N-terminal amino acids (Gly-Pro-Val-Asp) compared to the native molecule. We evaluated the ability of purified and 3C-cleaved lysins to kill log-phase P. aeruginosa strain PA01 (FIG. 14A). Log-phase PA01 cells were incubated with different lysin concentrations at 37° C. for 1 hour. All lysins demonstrated killing activity to some extent, however PlyPa01, PlyPa02, PlyPa91, and PlyPa96 had better activity compared to the others.

We analyzed a large group of lysins with close homology to PlyPa02 for lysins with improved killing activity, thus producing lysins PlyPa03, PlyPa09, PlyPa19, PlyPa21, PlyPa29 in a modified pET21-based plasmid. The lysins were purified, 3C-cleaved, and their killing activity against P. aeruginosa strain PA01 was determined as described above (FIG. 14B). These results demonstrated a substantial killing activity for all lysins, with a slight advantage for PlyPa03.

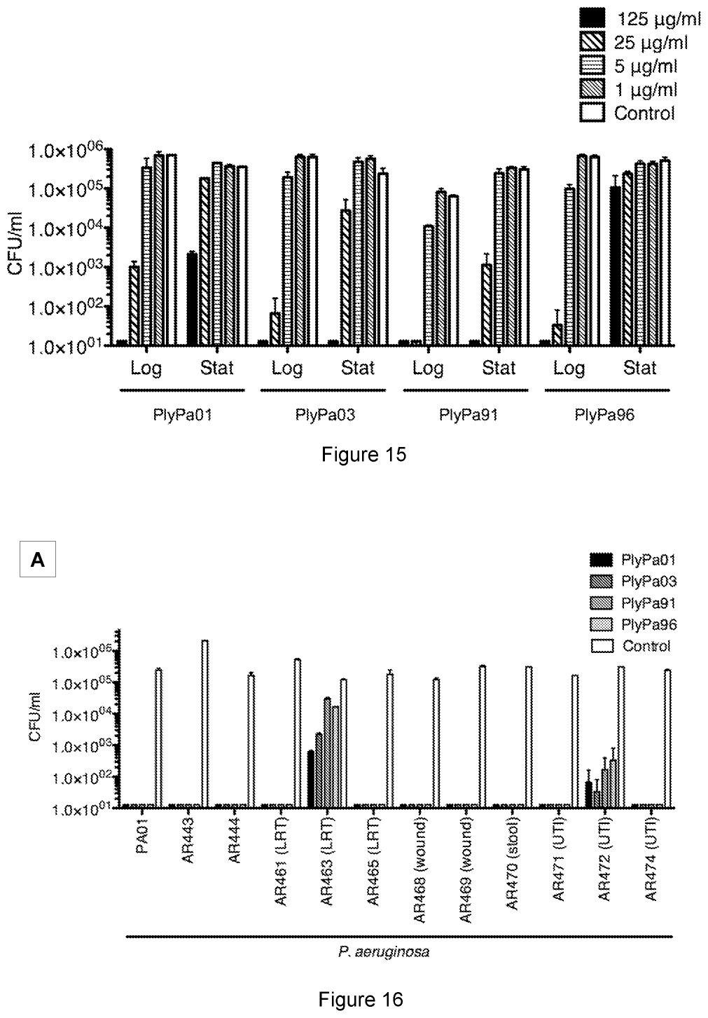

We next compared the activity of PlyPa01, PlyPa03, PlyPa91, and PlyPa96 against log-phase and stationary (grown overnight) P. aeruginosa cells (FIG. 15). In all cases, stationary bacteria were less susceptible to killing compared to log-phase cells. However, while the activity of PlyPa01 and PlyPa96 was markedly reduced when used against stationary bacteria, PlyPa03 and PlyPa91 retained substantial killing activity against these cells.

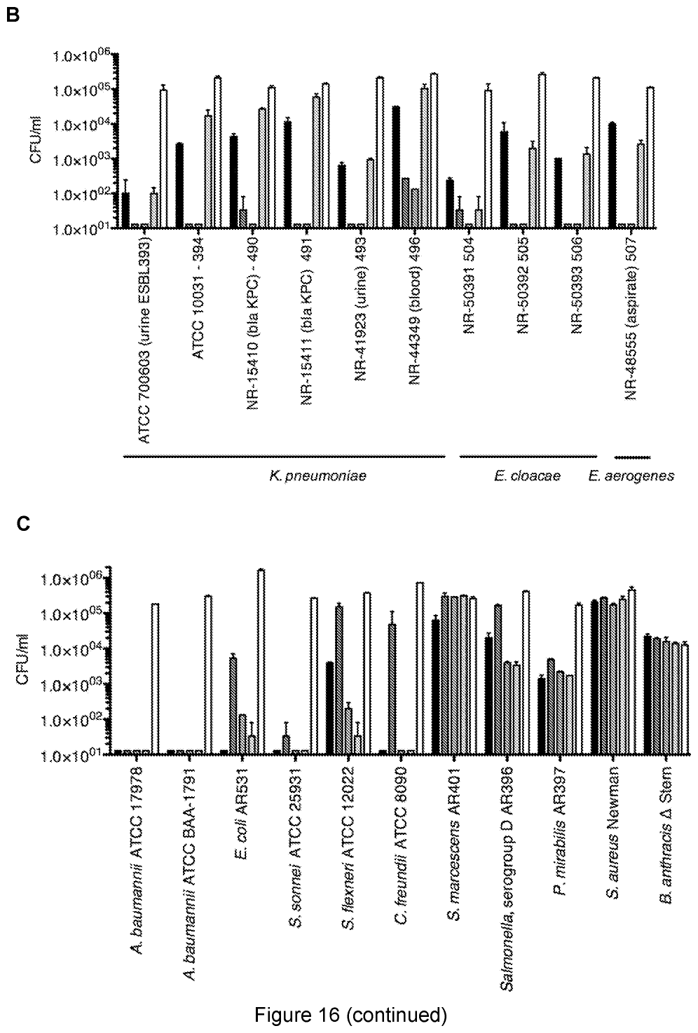

We next tested the killing activity of PlyPa01, PlyPa03, PlyPa91, and PlyPa96 at 100 μg/ml against recent clinical isolates of P. aeruginosa (FIG. 16A). Following 1 h incubation, all four enzymes reduced the colony count of most strains to below detection level. For AR463, a lower respiratory tract isolate, and AR472, a urinary tract infection isolate, the reduction in viable bacteria ranged between 1-4 logs. Both strains were completely eradicated by PlyPa03 at 250 μg/m1 concentration, and other lysins led to results ranging between complete eradication to substantial drop in viability at this concentration (FIG. 29).

We then tested the lysins against other Gram-negative pathogens. PlyPa03 and PlyPa91 had good killing activity against most Klebsiella and Enterobacter strains tested, resulting in 5-log kill in most cases, while PlyPa01 and PlyPa96 displayed only weak to moderate killing activity (FIG. 16B). PlyPa03 displayed relatively weak activity against E. coli, Shigella flexneri, and Citrobacter freundii, but PlyPa91 was active against these species, demonstrating a broader activity range (FIG. 16C). All enzymes had good activity against A. baumannii and Shigella sonnei, but only moderate to weak activity against Salmonella spp. and Proteus mirabilis. None of the enzymes had substantial activity against Serratia marcescens and the Gram-positive bacteria Staphylococcus aureus and Bacillus anthracis. These results revealed that despite the relatively broad range of the lysins tested, some level of species specificity does exist. Based on these results, we chose to proceed with PlyPa03 and PlyPa91 in further experiments.

### Characterization of PlyPa03 and PlyPa91

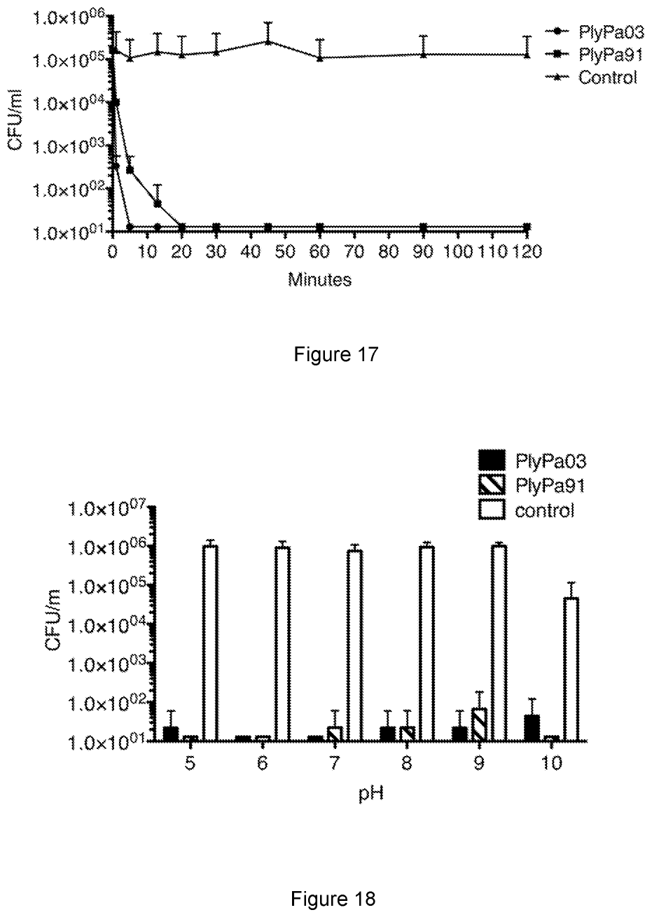

To evaluate the relative rate of P. aeruginosa killing by PlyPa03 and PlyPa91, we incubated P. aeruginosa PA01 cells with these lysins from one minute to two hours using 100 μg/ml each (FIG. 17). PlyPa03 rapidly killed P. aeruginosa, resulting in >2-log kill after one minute, and reduction to below detection level after 5 minutes. PlyPa91 had a slightly slower killing kinetics, resulting in 1-log kill after one minute, >2-log kill after 5 minutes, and reduction to below detection level after 20 minutes.

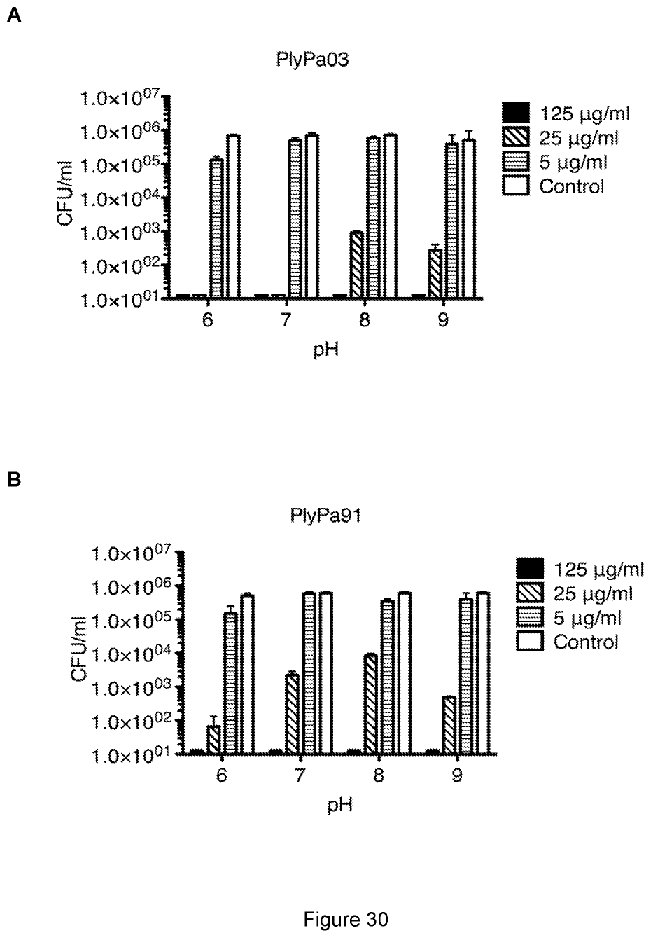

We next characterized the effect of pH, salt, and urea on the activity of PlyPa03 and PlyPa91. To determine the relative activity of the lysins in various pH conditions, log-phase P. aeruginosa cells were incubated with each of the lysins in buffer conditions ranging from pH 5.0 to 10.0 (FIG. 18). Both PlyPa03 and PlyPa91 effectively killed P. aeruginosa under all pH conditions tested. We further explored more subtle differences in activity at pH 6.0 to 9.0, by performing the experiments at various lysin concentrations (FIG. 29). Only slight differences in activity were observed among the different pH conditions, with PlyPa03 showing somewhat better activity at pH 6.0 and 7.0 compared to pH 8.0 and 9.0, and PlyPa91 showing somewhat better activity at pH 6.0 and 9.0, compared to pH 7.0 and 8.0, (FIG. 30).

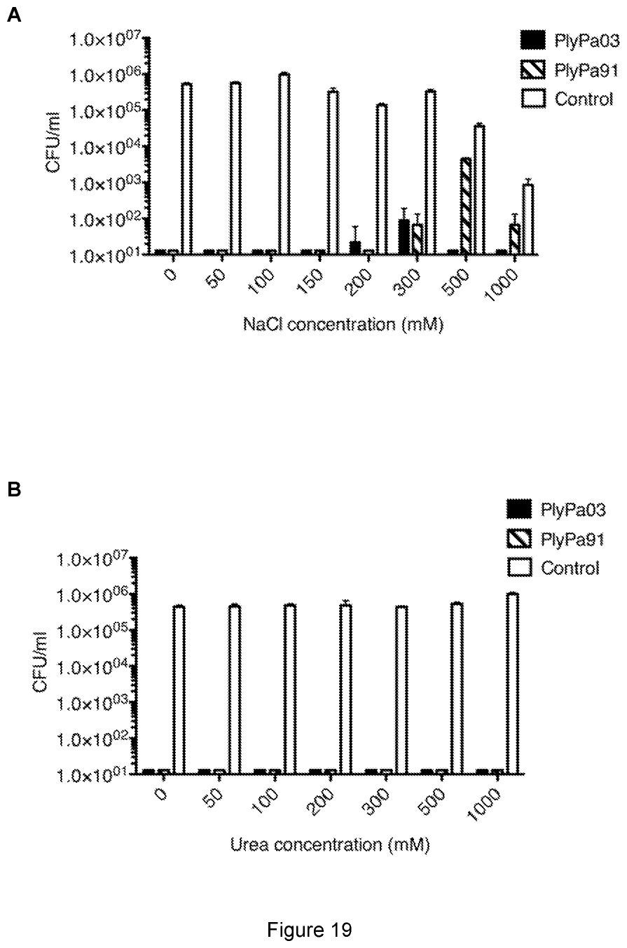

We next evaluated the effect of salt on the activity of PlyPa03 and PlyPa91 (FIG. 19A). In control samples, bacterial viability remained relatively constant up to 300 mM NaCl, but was slightly reduced at 500 mM NaCl, and substantially reduced at 1 M NaCl (preventing reliable estimation of lysin activity at this concentration). PlyPa03 remained active in NaCl concentrations as high as 500 mM, however the activity of PlyPa91 was substantially inhibited at 500 mM NaCl. We also evaluated the activity of PlyPa03 and PlyPa91 in urea. Both lysins were fully active in all urea concentrations tested up to 1 M. No reduction in bacterial viability was seen at these urea concentrations in the absence of lysins (FIG. 19B).

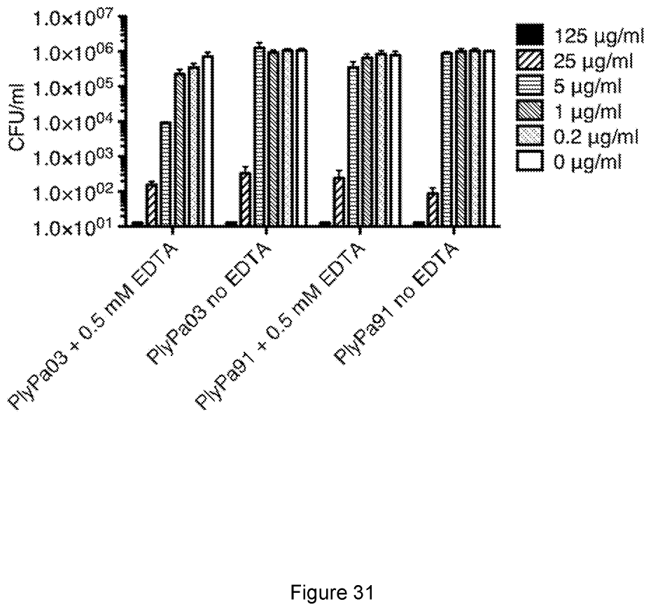

Chelation of divalent cations by EDTA destabilizes the outer membrane of Gram-negative bacteria, and can thus promote the translocation of externally applied lysins into the periplasm where they can degrade the cell wall peptidoglycan. We incubated P. aeruginosa cells with serially diluted PlyPa03 and PlyPa91 in the presence or absence of 0.5 mM EDTA, and determined the effect on killing activity (FIG. 31). Only a slight improvement in killing was observed for PlyPa03 in the presence of EDTA (at 5 μg/ml), and no improvement in killing was observed for PlyPa91. This may indicate that permeabilization of the outer membrane through chelation of divalent cations is not necessary for the activity of these lysins.

### Lysin Activity Against Pseudomonas Biofilm and in Serum and Surfactant

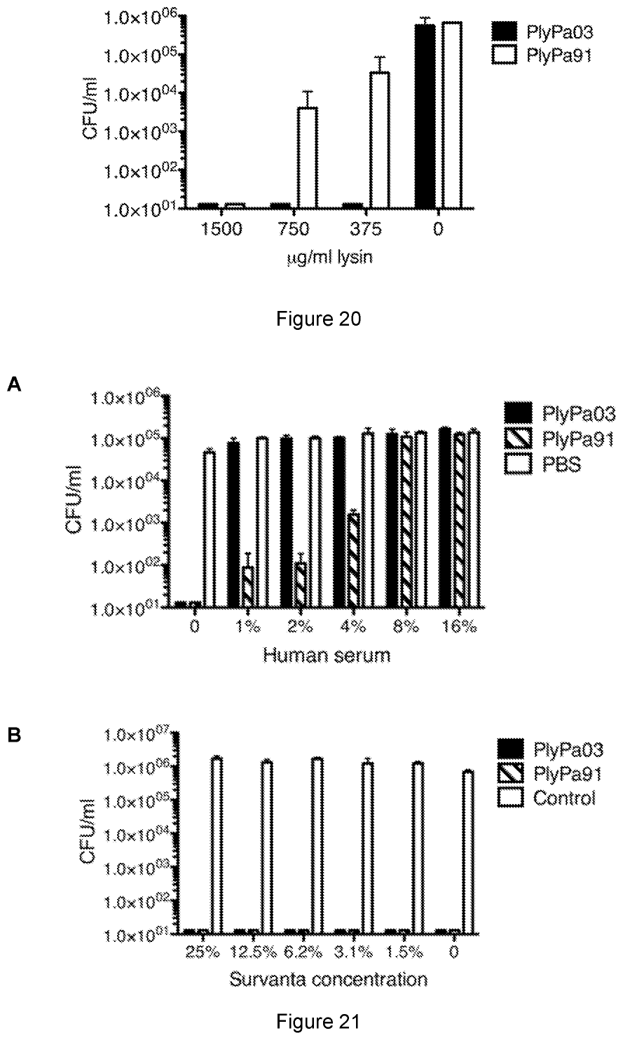

To test the effect of PlyPa03 and PlyPa91 on P. aeruginosa biofilm we used the MBEC Biofilm Inoculator 96-well plate system. Biofilms were grown for 24 h on the 96-peg lid, washed, and treated with different concentrations of PlyPa03, PlyPa91, or buffer control for 2 h at 37° C. Bacteria remaining on the pegs were dissociated by sonication and quantified by serial dilutions and plating. PlyPa03 completely eliminated P. aeruginosa biofilms at all concentrations tested, down to 0.375 mg/ml. Treatment with PlyPa91 resulted in >1-log CFU drop at 0.375 mg/ml, >2-log CFU drop at 0.75 mg/ml, and complete elimination of the biofilm at 1.5 mg/ml (FIG. 20). Thus, while both enzymes were effective in the elimination of P. aeruginosa biofilm, PlyPa03 performed substantially better.

Next we tested the activity of the lysins against P. aeruginosa in the presence of human serum (FIG. 21A). A very small amount of serum (1%) completely inhibited the killing activity of PlyPa03. On the other hand, PlyPa91 retained some activity at low serum concentrations, but it too was completely inhibited at 8% serum. As such, these lysins are not suitable for systemic use and would be better suited for topical applications. Nevertheless, PlyPa91 may be a better choice in topical environments where a certain amount of serum components may be expected.

An important potential use for lysins directed against P. aeruginosa is in the treatment of pneumonia. P. aeruginosa is among the most common causes for nosocomial pneumonia, an infection with a mortality rate as high as 30%. Lung surfactants are prominent components of the alveolar mucosa, and are critical for the maintenance of proper surface tension in the alveoli. Survanta is a concentrated mixture of bovine lung surfactants and artificial surfactants, and as such could be used to approximate the effect of lung surfactant on lysin activity. PlyPa03 and PlyPa91 were fully active against P. aeruginosa in the presence of all Survanta concentrations tested, up to 25% (FIG. 21B).

### Evaluation of Cytotoxic Effects of the Lysins

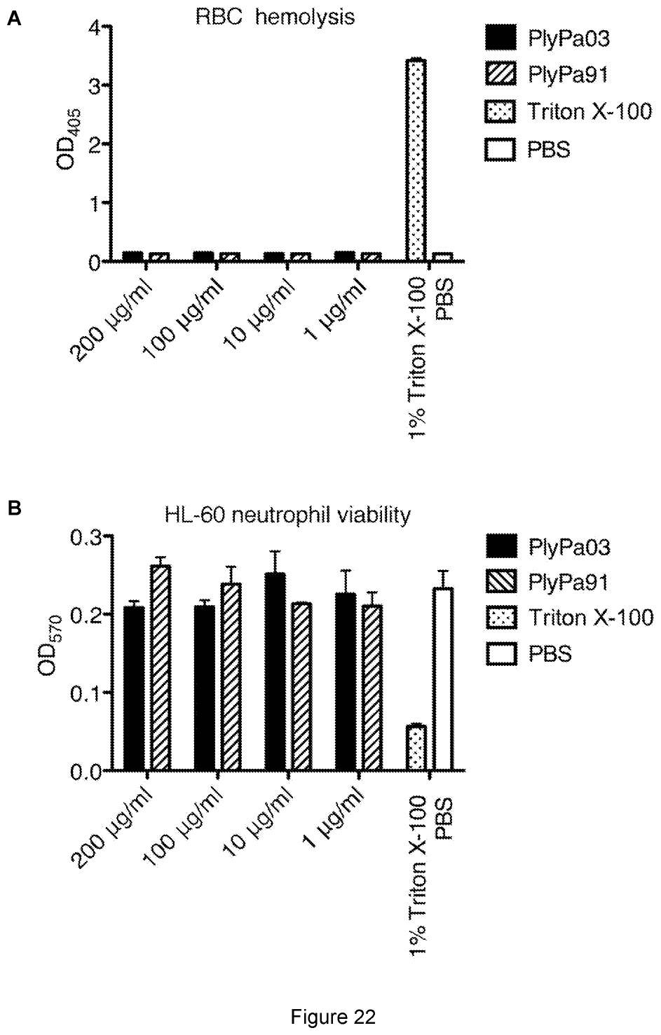

To evaluate the cytotoxicity of PlyPa03 and PlyPa91, we determined their effect on human red blood cells (RBCs). Human RBCs were incubated with PlyPa03 and PlyPa91 at concentrations ranging from 1 to 200 μg/ml for 4 hours, and release of hemoglobin was evaluated following removal of intact cells. No lysis of cells was observed at any concentration of either PlyPa03 or PlyPa91, while positive control 1% triton X-100 resulted in appreciable release of hemoglobin from the cells (FIG. 22). Thus, these lysins do not appear to have a lytic effect on RBC membranes.

### Evaluation of Lysin Efficacy in Murine Skin and Lung Models of Infection

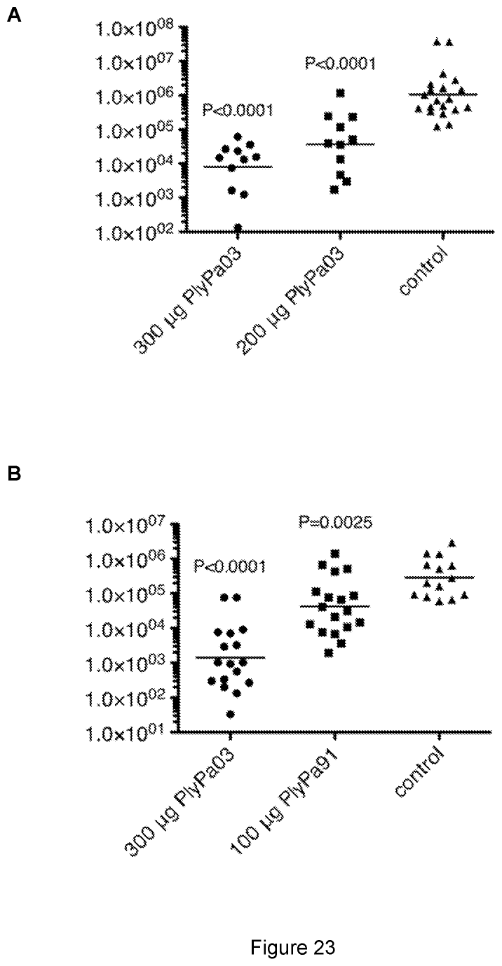

We tested PlyPa03 in a known mouse model of skin infection. Mice were shaved, depilated, and the top layers of the epidermis were removed by tape-stripping 15-20 times. P. aeruginosa cells were applied to the skin and allowed to establish infection for 20 h. The infected skin was treated with a single 200 μg or 300 μg dose of PlyPa03, or buffer control. Three hours later, the mice were euthanized, the infected skin was excised and homogenized, and the bacterial burden was evaluated by serial dilution and plating. Treatment of the infected skin with PlyPa03 resulted in a dose-dependent reduction in the P. aeruginosa, with the 300 μg dose leading to >2-log mean reduction in bacterial load (FIG. 23A). In a follow-up experiment we repeated the single 300 μg dose of PlyPa03, and included an additional group of mice treated with 100 μg PlyPa91. Results for the PlyPa03-treated group were in line with the previous experiment, resulting in >2-log mean reduction in bacterial load, while 100 μg PlyPa91 resulted in 1-log reduction in bacterial counts (FIG. 23B). Given that reduction in bacterial counts was reproducible and dose dependent, it is expected that higher doses and multiple repeat doses could lead to increased efficacy.

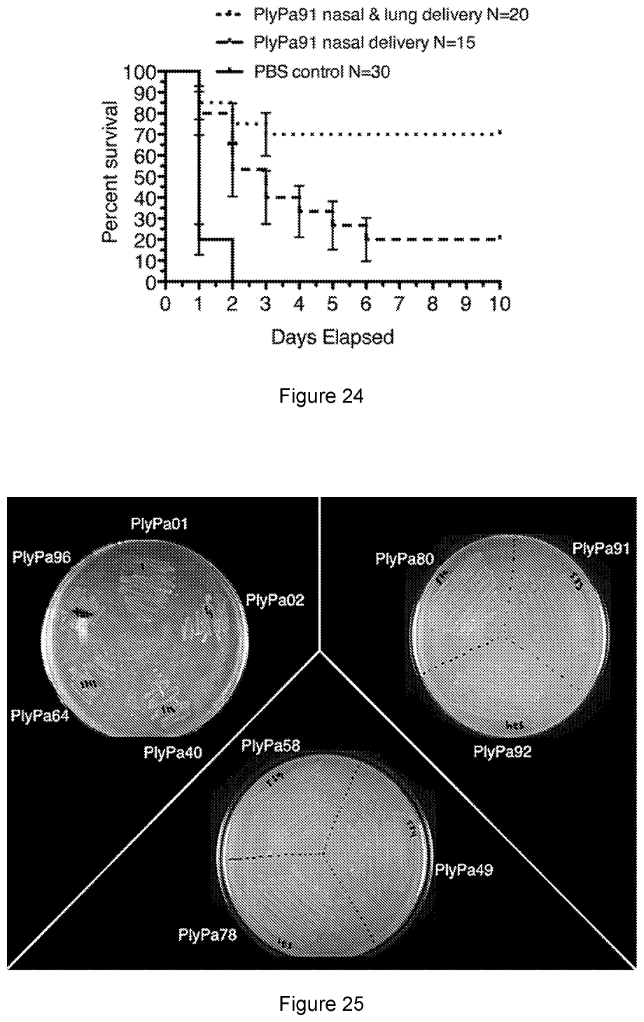

We next evaluated the efficacy of lysins in the treatment of P. aeruginosa pneumonia in a murine model infection. We chose to use PlyPa91 for these experiments given its higher resistance to serum components, some of which may be present in the lung mucosal exudate during infection. Female C57BL/6 mice were infected by intranasal application of 2×50 μl of 10⁸ CFU/ml log-phase P. aeruginosa PA01 to establish lung infection. The mice were treated at three and six hours post infection with 50 μl of 1.8 mg/ml PlyPa91 in PBS or PBS alone by either two intranasal instillations or by one intranasal and one intratracheal instillation. Survival of the mice was monitored daily for 10 days (FIG. 24). The majority of the mice in the control group died within the first 24 h, and remaining mice died by 48 h following infection. Mice treated with PlyPa91 in two intranasal instillations displayed a significant delay in death, with 20% of the mice surviving at day 10. Mice treated by a one intranasal and one intratracheal instillation displayed further reduction in death rate, with 70% of the mice surviving at day 10 (FIG. 24). Thus, PlyPa91 displayed significant protection of the mice in this model, and the route of delivery was important for treatment efficacy.

It will be apparent from the foregoing description in this Example that the disclosure provides two lysins that are highly active against P. aeruginosa. These lysins, PlyPa03 and PlyPa91, were effective against log phase and stationary bacteria, and were able to kill a wide range of Gram-negative organisms including clinical isolates of P. aeruginosa, A. baumannii, K pneumonia, and E. cloacae. Both lysins were active in a broad pH range, high urea concentrations, and in the presence of lung surfactants (Survanta).

Each of the lysins has a set of specific advantages. PlyPa03 was easier to produce in large quantities and displayed a potent killing activity, leading to a >5-log CFU reduction within 5 minutes, compared to 20 minutes for PlyPa91. Additionally, PlyPa03 was more resistant to salt, remaining active at 500 mM NaCl, while PlyPa91 was only active up to 300 mM NaCl (still well above the physiological salt concentration). PlyPa03 was also more effective against biofilms, an important trait given the role biofilms play in P. aeruginosa colonization and infection of the human host. Despite these advantages, PlyPa03 was highly sensitive to human serum, losing activity even in the presence of 1% serum, while PlyPa91 retained activity in low serum concentrations (up to 4%). Thus, while without intending to be bound by any particular theory, it is considered that neither enzyme should be used systemically, but PlyPa91 is likely better suited for use in environments where a small amount of serum components may be present. To verify this, in a mouse model of P. aeruginosa skin infection PlyPa03 demonstrated significant and dose-dependent killing of P. aeruginosa, showing potential in the treatment of topical P. aeruginosa infections. PlyPa91 was only tested at the 100 μg dose due to limitations in the amount of concentrated lysin available. This still resulted in over 1-log kill, which was in line with the PlyPa03 results. We chose PlyPa91 for use in the murine pneumonia model to test its suitability in mucosal environments based on its higher resistance to serum components. In this model, PlyPa91 protected mice from death following P. aeruginosa delivery to the lungs. The route of delivery had a significant effect on the survival of the mice. Where 70% of mice treated with a combination of intranasal and intratracheal instillations were protected, only 20% of mice treated with two intranasal instillations survived, despite a similar amount of lysin used. Thus, in a clinical setting, an effective delivery system like aerosol inhalation combined with repeated dosing, could greatly contribute to treatment efficacy, and such approaches are encompassed by the present disclosure.

While the present disclosure has been described with reference to the specific embodiments thereof, it should be understood by those skilled in the art that various changes may be made and equivalents may be substituted without departing from the true spirit and scope of the disclosure. In addition, many modifications may be made to adopt a particular situation, material, composition of matter, process, process step or steps, to the objective spirit and scope of the present disclosure. All such modifications are intended to be within the scope of the claims appended hereto.

1. A pharmaceutical composition for killing Gram-negative bacteria comprising an effective amount of at least one isolated or recombinant lysin polypeptide comprising one amino acid sequence of Table 1, or variants thereof having at least 80% identity to the least one polypeptide of Table 1, and wherein optionally a recombinant lysin polypeptide comprises an additional amino acid sequence that is a purification tag, or an antimicrobial peptide.

2. The pharmaceutical composition of claim 1, wherein the Gram-negative bacteria are selected from Klebsiella pneumonia, Enterobacter bacteria, Pseudomonas, and combinations thereof.

3. The pharmaceutical composition of claim 1, wherein the Gram-negative bacteria are the Klebsiella pneumonia, the Enterobacter, or a combination thereof.

4. The pharmaceutical composition of claim 2, wherein the Gram-negative bacteria comprise the Pseudomonas, and are optionally Pseudomonas aeruginosa.

5. The pharmaceutical composition of claim 3, wherein the at least one lysin polypeptide comprises the amino acid sequence:
(SEQ ID NO: 25)
MAWGAKVSKEFKLKVIEVCERLEINPDYLMSCMAFETGETFSPNV
RNPNGSATGLIQFMSNTARSLGTTTNELADMTSVEQMDYVEKYFK
PYAGKIKTIEDVYMVIFCPRAVGKPDSYILYDEGRSYNDNKGLDL
NKDNAITKYEAGFKVREKLKLGMKEGYRG.
(PlyKp104)

6. The pharmaceutical composition of claim 4, wherein the at least one lysin polypeptide comprises the amino acid sequence:
(SEQ ID NO: 66)
MKGKVIGGSAAAVIALAAAALVKPWEGYSPTPYIDMVGVATHCY
GDTSRADKAVYTEQECAEKLNSRLGSYLTGISQCIKVPLREREW
AAVLSWTYNVGVGAACRSTLVGRINAGQPAASWCPELDRWVYAG
GKRVQGLVNRRAAERRMCEGRS;
(P1yPa91)
or wherein the at least one lysin polypeptide comprises the amino acid sequence:
(SEQ ID NO: 64)
MAWSAKVSQAFCDRVIWIAASLGMPADGADWLMACIAWETGETF
SPSVRNGAGSGATGLIQFMPATARGLGTTTDELARMTPEQQLDY
VYRYFLPYRGRLKSLADTYMAILWPAGIGRALDWALWDSTSRPT
TYRQNAGLDINRDGVITKAEAAAKVQAKLDRGLQPQFRRAAA.
(P1yPa103)

7. A method of killing Gram-negative bacteria comprising the step of contacting the bacteria with the pharmaceutical composition of claim 1.

8. The method of claim 7, wherein the Gram-negative bacteria are present in an infection of an individual.

9. The method of claim 8, wherein the Gram-negative bacteria are selected from Klebsiella, Enterobacter bacteria, Pseudomonas, and combinations thereof.

10. The method of claim 9, wherein the Gram-negative bacteria are Klebsiella pneumonia, the Enterobacter bacteria, or a combination thereof.

11. The method of claim 10, wherein the Gram-negative bacteria are the Klebsiella pneumonia.

12. The method of claim 8, wherein the Gram-negative bacteria are the Pseudomonas.

13. The method of claim 12, wherein the Pseudomonas comprise Pseudomonas aeruginosa.

14. The method of claim 7, wherein the Gram-negative bacteria are resistant to at least one antibiotic.

15. The method of claim 7, wherein the Gram-negative bacteria are any of: in a biofilm; in an infection of the skin of the individual; in an infection of mucosa of the individual, wherein optionally the mucosa is present in the lungs of the individual; in a wound of the individual; or in contact with sera of the individual.

16-21. (canceled)

22. A recombinant DNA molecule comprising a DNA sequence that encodes a lysin polypeptide from Table 1, or a variant thereof having at least 80% identity to the lysin polypeptide of Table 1.

23. The recombinant DNA molecule of claim 22, wherein the DNA sequence encodes: PlyKp104 comprising the sequence of SEQ ID NO:25; or PlyPa91 comprising the amino acid sequence of SEQ ID NO:66, or PlyPa103 comprising the amino acid sequence of SEQ ID NO:64.

24-27. (canceled)

28. A method for reducing or controlling Gram-negative bacteria in a mammal which has an infection of the Gram-negative bacteria, the method comprising contacting the skin of the mammal, and/or or introducing into the mammal, an effective amount of the pharmaceutical composition of claim 1, such that the number of Gram-negative bacteria on or in the mammal are reduced.

29. The method of claim 28, wherein the mammal is a human.

30. The method of claim 28, wherein the wherein the pharmaceutical composition comprises at least one lysin polypeptide that comprises the amino acid sequence:
(SEQ ID NO: 25)
MAWGAKVSKEFKLKVIEVCERLEINPDYLMSCMAFETGETFSPN
VRNPNGSATGLIQFMSNTARSLGTTTNELADMTSVEQMDYVEKY
FKPYAGKIKTIEDVYMVIFCPRAVGKPDSYILYDEGRSYNDNKG
LDLNKDNAITKYEAGFKVREKLKLGMKEGYRG,
(PlyKp104)
or
(SEQ ID NO: 66)
MKGKVIGGSAAAVIALAAAALVKPWEGYSPTPYIDMVGVATHCYG
DTSRADKAVYTEQECAEKLNSRLGSYLTGISQCIKVPLREREWAA
VLSWTYNVGVGAACRSTLVGRINAGQPAASWCPELDRWVYAGGKR
VQGLVNRRAAERRMCEGRS;
(P1yPa91)
or:
(SEQ ID NO: 64)
MAWSAKVSQAFCDRVIWIAASLGMPADGADWLMACIAWETGETF
SPSVRNGAGSGATGLIQFMPATARGLGTTTDELARMTPEQQLDY
VYRYFLPYRGRLKSLADTYMAILWPAGIGRALDWALWDSTSRPT
TYRQNAGLDINRDGVITKAEAAAKVQAKLDRGLQPQFRRAAA,
(P1yPa103)
or a combination of the P1yKp104, P1yPa91, and the P1yPa103.


SEQUENCE LISTING (67 sequences)

SEQ ID NO: 1 - protein, 146 aa, Klebsiella pneumoniae bacteriophage
MEMSNNGINMLKGFEGCRLAAYQDSVGVWTIGYGWTQPVNGVPVGKGMTITQDTADSLLRSGLVQYEKGVTGLVKVTINQNQFDALVDFAYNLGVKALEGSTLLKKLNAGDYAGAAAEFPKWNKAGGKVLPGLVKRREAERTLFLA

SEQ ID NO: 2 - protein, 146 aa, Klebsiella pneumoniae bacteriophage
MQTSEKGISLIKEFEGCKLNAYQDSVGVWTIGYGWTQPVDGKPIRAGMTIKQETAERLLKTGLVSYESDVSRLVKVGLTQGQFDALVSFTYNLGARSLSTSTLLRKLNAGDYAGAADEFLRWNKAGGKILNGLTRRREAERALFLS

SEQ ID NO: 3 - protein, 150 aa, Klebsiella pneumoniae bacteriophage
MANQPQHTGDAGVALIKSFEGLRLEKYRDAVGKWTIGYGHLILPNENFPRPITEAEADALLRKDLQTSERGVHRLVTVDLDQDQFDALVSFTFNLGAGNLQSSTLLKLLNQGEYTQAADQFLRWNKAGGRVLPGLTRRREAERALFLQAG

SEQ ID NO: 4 - protein, 149 aa, Klebsiella pneumoniae bacteriophage
MQTSPEGIALIKGFEGCRLTAYPDPGTGGVPWTIGYGWTLPIDGKPVRPGMTIDQVTADRLLKTGLVSYESDVLKIVKVKLNQNQFDALVSFAYNVGSRALSTSTLLKKLNAGDIKGAADEFLRWNKAGGKVLNGLTRRREAERALFLS

SEQ ID NO: 5 - protein, 149 aa, Klebsiella pneumoniae bacteriophage
MQISNNGIALIKRFEGCRLTAYPDPGTGGDPWTIGYGWTGKVDGKPIRPGMKIDEATADRLLRTGVVSFDQAVSKMLKVTVTQNQYDALVSLAYNIGTRALSTSTLMKKLNAGDVKGAADEFLRWNRSGGKVMAGLTNRRKAEREVFLS

SEQ ID NO: 6 - protein, 149 aa, Klebsiella pneumoniae bacteriophage
MQISNNGIALIKRFEGCRLTAYPDPGTGGGPWTIGYGWTGKVDGKPIKPGMKIDDATADRLLRTGVVSFDQAVSKMLKVSVTQNQYDALVSLAYNIGTRALSTSTLMKKLNAGDVKGAADAFLSWNRSGGKVMAGLTNRRKAEREVFLS

SEQ ID NO: 7 - protein, 159 aa, Klebsiella pneumoniae bacteriophage
MQISDNGIALIKGFEGCRLTAYPDPGTGGDPWTIGFGWTGKVDGKPIKPGMKIDDATADRLLRTGVVSFDLAVSKMLKVSVTQNQYDALVSLAYNIGTRALSTSTLMKKLNAGDVKGAADEFLRWNKSGGKAMSGLTNRRKAEREVFLSKTRGSYELSH

SEQ ID NO: 8 - protein, 164 aa, Klebsiella pneumoniae bacteriophage
MNPTLRNKLIGAIAGGSGAIVIASVMLGNADGLEGRRYYAYQDVVGVWTVCDGHTGTDIRRGHRYTDRECDNLLKADLRKVASAIDPLIKVSIPDPTRAALYSFTYNVGSGAFASSTLLKKLNAGDVPGACKELQRWTYAGGKQWKGLISRREIEREVCLWGQK

SEQ ID NO: 9 - protein, 167 aa, Klebsiella pneumoniae bacteriophage
MVMSPKLRNSVLAAVGGGAIAIASALITGPTGNDGLEGVRYKPYQDVVGVWTVCYGHTGKDIMLGKTYTESECRALLNKDLNIVARQINPYIQKPIPETMRGALYSFAYNVGAGNLQTSTLLRKINQGDQKGACDQLRRWTYAKGKQWKGLVTRREIEREVCLWGQK

SEQ ID NO: 10 - protein, 145 aa, Klebsiella pneumoniae bacteriophage
MRISSNGVVRLKGEEGERLSAYLDSRGIPTIGVGHTGTVDGKPVVIGMVISQNKSTELLLQDIQWVEKAINSSVKTPLTQNQYDALCSLVFNIGATAFYGSTVLKRVNQKDYTAAADAFLMWKKAGKDQEILLPRRRRERALFLS

SEQ ID NO: 11 - protein, 164 aa, Klebsiella pneumoniae bacteriophage
MNPTLRNKLIGAIAGGSGAIAIASVMLGNADGLEGRRYYAYQDVVGVWTVCDGHTGTDIRRGHRYTDRECDSLLKADLRKVASAIDPLIKVRIPDPTRAALYSFTYNVGSGAFASSTLLKKLNAGDVPGACKELQRWTYAGGKQWKGLITRREIEREVCEWGQK

SEQ ID NO: 12 - protein, 164 aa, Klebsiella pneumoniae bacteriophage
MNPTLRNKLIGAIAGGSGAIAIASVMLGNADGLEGRRYYAYQDVVGVWTVCDGHTGTDIRRGHRYTDRECDNLLKADLRKVASAIDPLIKVRLPAPTRAALYSFTYNVGSGAFASSTLLKKLNAGDVPGACKELQRWMYAGGKQWKGLITRREIEREVCEWGQK

SEQ ID NO: 13 - protein, 146 aa, Klebsiella pneumoniae bacteriophage
MQTSEKGIALIKEFEGCKLTAYQDSVGVWTIGYGWTHPVDGKPIRAGMTIKQETAERLLKTGLVSYECDVSRLVKVGLTQGQFDALVSFTYNLGARSLSTSTLLRKLNAGDYAGAADEFLRWNKAGGKVLNGLTRRREAERALFLS

SEQ ID NO: 14 - protein, 149 aa, Klebsiella pneumoniae bacteriophage
MQISDEGIALIKGFEGCRLTAYPDPGTGGAPWTIGYGWTLPVDGKPVRPGMTIDQATADRLLKIGLVGYENDVLKIVKVKLTQGQFDALVSFAYNIGSRALSTSTLLKKLNAGDIKDAADEFLRWNKAGGKVLNGLTRRREAERALFLS

SEQ ID NO: 15 - protein, 145 aa, Klebsiella pneumoniae bacteriophage
MQVSDNGIVFLKNEEGEKLTGYPDSRGIPTIGVGHTGKVNGVPVSVGMKITSEQSSELLKDDLSWVEDSIANYVKSPLNQNQYDALCSFIFNIGAPAFEGSTMLKLLNKSDYVGASGEFPKWKRAGNDPDILLPRRMREQALFLS

SEQ ID NO: 16 - protein, 145 aa, Klebsiella pneumoniae bacteriophage
MQISSNGITKLKREEGERLKAYPDSRGIPTIGVGHTGNVDGKPVTLGMTITSDKSSELLKADLRWVEDAISSLVRVPLTQNQYDALCSLIFNIGKSAFAGSTVLRQLNLKNYQAAADAFLMWKKAGKDTEILLPRRQRERALFLS

SEQ ID NO: 17 - protein, 164 aa, Klebsiella pneumoniae bacteriophage
MNPTLRNKLIGAIAGGSGAIAIASVMLGNADGLEGRRYYAYQDVVGVWTVCDGHTGSDIRRGHRYSDKECDNLLKSDLRKVANAIDPLIKVRIPDPTRAALYSFTYNVGSGAFASSTLLKKLNAGDVPGACKELQRWTYAGGKQWKGLITRREIELEVCEWGQK

SEQ ID NO: 18 - protein, 165 aa, Klebsiella pneumoniae bacteriophage
MNQPLRKYVLSAVGGGAIAIASALITGPTGNDGLEGVRYQPYQDVVGVWTVCYGHTGKDIMLGNTYTKSECDALLDKDLNTVARQINPYIKKPIPETMRGALYSFAYNVGAGSFQTSTLLRKINQGDSKGACEQLRVWIYAGKKVWKGLVTRREIEREVCLWGQK

SEQ ID NO: 19 - protein, 169 aa, Klebsiella pneumoniae bacteriophage
MNPSIVKRCLVGAVLAIAATLPGFQSLHTSVEGLKLIADYEGCRLQPYQCSAGVWTDGIGNTSGVVPGKTITERQAAQGLITNVLRVERALDKCVAQPMPQKVYDAVVSFAFNVGTGNACSSTLVKLLNQRRWADACHQLPRWVYVKGVFNQGLDNRRAREMAWCLKGA

SEQ ID NO: 20 - protein, 164 aa, Klebsiella pneumoniae bacteriophage
MAMSPALRNSIVAALGTGAIGIATVMVSGKSGLEGREHYPYKDIVGIVTVCDGYTGSDIVWGKYYSDKECDALTRKDMTRIAAQVNPHIKVPTTETQRAAIYSFAYNVGSTAAINSTLLKKLNSKDYSGACSELKRWVYAGGKKWKGLMNRRDVEYEVCTWSQK

SEQ ID NO: 21 - protein, 169 aa, Klebsiella pneumoniae bacteriophage
MSSIVKRCSVAAVLALAALLPDFRLLHTSPDGLALIADLEGCRLAPYQCSAGVWTSGIGHTAGVVPKRDITEREAAANLVADVLNTERRLAVCVPVTMPQPVYDALVSFSFNVGTGAACRSTLVSYIKRHQWWQACDQLSRWVYVNGERSTGLENRRQRERAYCLKGVK

SEQ ID NO: 22 - protein, 175 aa, Klebsiella pneumoniae bacteriophage
MTNKVKFSAAMLALLAAGATAPELFDQFMSEKEGNALVAVVDPGGVWSLCHGVIFIDGKRVVKGMTATESQCRKVNAIERDKALSWVDRNINVPLTEPQKVGIASFCPYNIGPGKCFPSTFYKRINAGDRKGACEAIRWWIKDGGKDCRIRSNNCYGQVTRRDQESALTCWGIDQ

SEQ ID NO: 23 - protein, 178 aa, Klebsiella pneumoniae bacteriophage
MSNKAKFSAAMLVLLAAGASAPVLFDQFIGEREGNSLTAVIDPGGVWSICRGVTRIDGRPVVKGMNLTQSQCDHYNAIERDKALAWVQKNVHVPLTEPQKVGIASFCPYNIGPGKCFPSTFYRKLNAGDRKGACAEIRRWIFDGGRDCRLTKGQANGCYGQVDRRDQESALTCWGLYE

SEQ ID NO: 24 - protein, 177 aa, Klebsiella pneumoniae bacteriophage
MASLKTKLSAAMLGLIAAGASAPTLMDQFLDEKEGNSLTAYRDGSQGIWTICRGATRIDGKPVTQGMKLTQAKCDEVNDIERDKALAWVDRNIRVPLTPPQKVGIASFCPYNIGPGKCFPSTFYQRINAGDRKGACEAIRWWIKDGGKDCRIRSNNCYGQVTRRDQESALTCWGIDQ

SEQ ID NO: 25 - protein, 164 aa, Klebsiella pneumoniae bacteriophage
MAWGAKVSKEFKLKVIEVCERLEINPDYLMSCMAFETGETFSPNVRNPNGSATGLIQFMSNTARSLGTTTNELADMTSVEQMDYVEKYFKPYAGKIKTIEDVYMVIFCPRAVGKPDSYILYDEGRSYNDNKGLDLNKDNAITKYEAGFKVREKLKLGMKEGYRG

SEQ ID NO: 26 - protein, 176 aa, Klebsiella pneumoniae bacteriophage
MANLKTKLSSAMLALIAAGASAPVLMDQFLNEKEGKSLTSYRDGAGIWTICRGVTQVDGRPVTQGMKLTQAKCDQVNAVERNKALAWVDQNVRVPLTPPQKVGIASFCPYNIGPGKCFPSTFYRKLNAGDRKGACAEIRRWIFDGGKDCRVRSNNCYGQVSRRDQESALACWGIDE

SEQ ID NO: 27 - protein, 199 aa, Klebsiella pneumoniae bacteriophage
MQISDNGIALIKGFEGCRLTAYPDPGTGGDPWTIGFGWTGKVDGKPIKPGMKIDDATADRLLRTGVVSFDLAVSKMLKVSVTQNQYDALVSLAYNIGTRALSTSTLMKKLNAGDVKGAADEFLRWNKSGGKAMSGLTNRRKAEREVFLSKTRGSYELSHGTGGGSGGGSGGGDHECHYRIKPTFRRLKWKYKGKFWCPS

SEQ ID NO: 28 - protein, 209 aa, Klebsiella pneumoniae bacteriophage
MQISDNGIALIKGFEGCRLTAYPDPGTGGDPWTIGFGWTGKVDGKPIKPGMKIDDATADRLLRTGVVSFDLAVSKMLKVSVTQNQYDALVSLAYNIGTRALSTSTLMKKLNAGDVKGAADEFLRWNKSGGKAMSGLTNRRKAEREVFLSKTRGSYELSHGTGGGSGGGSGGGLLGDFFRKSKEKIGKEFKRIVQRIKDFLRNLVPRTES

SEQ ID NO: 29 - protein, 190 aa, Klebsiella pneumoniae bacteriophage
MQISDNGIALIKGFEGCRLTAYPDPGTGGDPWTIGFGWTGKVDGKPIKPGMKIDDATADRLLRTGVVSFDLAVSKMLKVSVTQNQYDALVSLAYNIGTRALSTSTLMKKLNAGDVKGAADEFLRWNKSGGKAMSGLTNRRKAEREVFLSKTRGSYELSHGTGGGSGGGSGGGRKKTRKRLKKIGKVLKWI

SEQ ID NO: 30 - DNA, 441 bp, Klebsiella pneumoniae bacteriophage
gtggagatgagcaataacggcatcaacatgctgaaaggttttgaagggtgcaggctggccgcttatcaggattctgtaggcgtctggacgatcggttatggatggactcaacccgtcaacggcgtgccggttggcaagggcatgaccattacgcaggacactgccgatagcctgttgcgtagcggtctggtgcaatatgaaaaaggcgttacggggctcgttaaagtcaccatcaatcaaaatcagttcgatgcgctggttgattttgcctacaacctgggcgtaaaggcgctggaaggatccacgctgctgaaaaagctgaatgctggcgattacgccggggctgcggctgagtttccaaaatggaataaagcaggtggcaaggtgttgccggggctggttaagcgtcgggaagccgagcgtacgttatttctggcctga

SEQ ID NO: 31 - DNA, 441 bp, Klebsiella pneumoniae bacteriophage
atgcaaaccagcgaaaagggtatttccctgatcaaagagttcgaaggctgcaagcttaacgcctaccaggacagcgtcggtgtatggacgattggctatggctggactcagcctgtcgacggcaaaccaatccgcgccgggatgacgattaagcaggagacagcagagcgcctgctgaagaccggactggtcagctacgaaagcgatgtgtcccgcctggtaaaagttggcctgactcaggggcaattcgatgccctggtatcgttcacgtacaacctcggcgcccggtcactgtcgacatctaccctgctgcgaaaactcaacgcaggtgattacgctggcgctgccgatgagttcctgcgctggaataaagctggtggcaagatcctgaatggtctgacccgtcggcgtgaggcggagcgcgctctgttcctgtcgtga

SEQ ID NO: 32 - DNA, 453 bp, Klebsiella pneumoniae bacteriophage
atggcgaatcaaccgcaacacaccggcgatgctggcgtcgcattaatcaaatcttttgaagggctacggctggagaagtatcgcgatgccgtcggcaagtggaccattggctacgggcacctgatcctgccgaacgagaactttccgcgcccgattaccgaagcggaggctgacgcgctgctgcgcaaggatttgcagacgagcgagcgcggcgtgcaccggctggtgacggtcgatctcgaccaggatcagttcgacgcgctggtgtcgtttaccttcaacctcggcgccgggaatttgcagagctcgacgctgctcaagttgttaaatcaaggcgaatatacgcaggccgccgaccagtttctgcgctggaacaaagcgggcggcagagtgctgcccggcctgacacggcggcgtgaagcggagcgggcgctgtttttgcaggcgggttag

SEQ ID NO: 33 - DNA, 450 bp, Klebsiella pneumoniae bacteriophage
atgcaaaccagtcctgaaggaattgcactgataaaagggtttgaaggctgccggctgaccgcataccccgatccgggaactggtggtgtgccgtggacaattggctatggctggaccctccccatcgacggtaagccggtaaggccgggaatgactattgaccaggtaacagcggatcgtctgcttaaaaccgggctggtgagctacgagagcgatgtgctgaagatcgttaaagtgaagctgaatcagaatcaatttgatgccctggtatcgttcgcctacaacgtcggctcccgcgcattatcaacttcaactctgctgaaaaagctcaatgctggcgacatcaaaggcgctgctgatgagtttctgcgctggaataaagctggcggcaaagtcctgaatgggctgacccgccgacgtgaggcggagcgcgctctgttcctgtcgtga

SEQ ID NO: 34 - DNA, 450 bp, Klebsiella pneumoniae bacteriophage
atgcaaatcagtaataacggtatcgcgctgattaagcgatttgagggttgtcggttaaccgcatatcccgacccgggcacaggtggtgatccctggacgattggctacggctggacgggaaaagtagacgggaagcctatcaggcccggaatgaagattgacgaagcaacggcggatcgtctgctgcgcactggcgtagtgagctttgatcaggcggtaagcaagatgctcaaagttaccgttacccagaaccagtacgacgcgcttgtgtcgctggcctacaacatcggtactcgagcgttatccacatcaacgctgatgaagaagctgaatgcaggtgatgtgaaaggcgcggctgatgagttccttcgctggaaccggtcaggcggcaaggtaatggctggcctcactaatcgccgcaaggcagagcgagaagtctttttatcgtga

SEQ ID NO: 35 - DNA, 450 bp, Klebsiella pneumoniae bacteriophage
atgcaaatcagtaataacggtattgcgctgattaagcgatttgagggttgcaggttaactgcatatcccgacccgggcaccggcggtggtccctggacgattggctacggctggacggggaaagtagacggaaagcctatcaagcccggaatgaagattgacgacgcaacggcggatcgcctgctgcgcactggcgtggtgagctttgaccaggcggtaagcaagatgctcaaggtctccgttacccagaaccagtatgacgcgcttgtgtcgctggcctacaacatcggtacgcgagcgttatctacatcaacgctgatgaagaagctgaatgcaggtgatgtgaaaggtgccgctgacgcattcttgagctggaaccgttcaggcggcaaggtaatggctggcctcaccaatcgtcgcaaggcagagcgggaagtctttttatcgtga

SEQ ID NO: 36 - DNA, 480 bp, Klebsiella pneumoniae bacteriophage
atgcagataagcgataacggcatcgcactgattaaggggtttgaaggatgtcgattaaccgcatacccggacccgggcaccggcggtgatccctggacgattggtttcggctggacggggaaagtagacggcaagcctatcaagccgggaatgaagattgacgatgcgacagcggatcgcctgctgcgcactggcgtggtgagctttgacctggcggtaagcaagatgctcaaagtttccgtcacccagaatcagtacgacgcgcttgtgtcgctggcctataacatcggtacgcgagcgctatccacctcaacgctgatgaaaaagctgaatgcaggtgatgtgaaaggcgcagctgatgagttccttcgctggaataaatcaggcgggaaagcaatgtctgggctaaccaatcgccgcaaggcagagcgagaagtatttttatcgaaaacacggggaagttatgaactatctcattaa

SEQ ID NO: 37 - DNA, 495 bp, Klebsiella pneumoniae bacteriophage
atgaacccgacgctgaggaataagctgattggtgcgatcgccggcggttcgggcgcgatagtcattgcttccgtcatgcttggtaatgctgacggcctggaaggaaggcgttattacgcctatcaggatgttgtcggcgtctggactgtttgtgatggtcacactggcaccgatattcgccgcggccaccgctacaccgacagagaatgcgacaacctgctgaaggctgatctgcggaaggtggcaagcgccattgacccgcttatcaaagtcagcattcctgaccccacccgcgccgcgctttactcattcacctacaacgttggctctggagctttcgccagttccacgctgctgaagaaactgaatgctggagatgtgccgggcgcgtgcaaggaactgcagcgctggacatacgctggcgggaagcagtggaaaggccttatctcaaggcgcgagattgagcgcgaagtttgtctgtgggggcagaaatga

SEQ ID NO: 38 - protein, 504 aa, Klebsiella pneumoniae bacteriophage
ATGGTAATGTCACCAAAGCTCAGGAATAGCGTTCTTGCTGCCGTTGGTGGTGGTGCTATTGCCATTGCGTCGGCTCTCATCACCGGGCCAACCGGCAATGATGGTCTGGAGGGAGTGAGGTATAAGCCGTATCAGGATGTTGTAGGCGTCTGGACAGTCTGCTATGGCCACACTGGCAAAGATATCATGCTCGGTAAAACCTACACCGAGTCAGAGTGTCGCGCGCTGCTCAACAAAGACCTGAACATCGTCGCACGCCAGATCAACCCGTACATCCAGAAGCCGATCCCCGAAACAATGCGTGGGGCTCTGTACTCGTTTGCTTATAACGTAGGCGCCGGAAACTTACAGACCTCCACTCTGCTGCGCAAAATCAACCAGGGCGACCAGAAAGGTGCATGCGACCAGTTGCGCCGCTGGACTTATGCCAAAGGAAAGCAGTGGAAAGGCCTGGTAACTCGCCGCGAGATTGAGCGCGAAGTTTGTCTTTGGGGGCAGAAATGA

SEQ ID NO: 39 - DNA, 438 bp, Klebsiella pneumoniae bacteriophage
atgcgaatcagcagtaatggcgttgtccggctcaaaggcgaagaaggcgagcgcctcagtgcttatctggatagtcgcggcatcccaaccataggcgttggccacacaggaacagtcgacggcaagccagtggtgatcggtatggttatcagccagaacaaatcgactgagctgctgctgcaggatatccagtgggtagagaaggcgatcaacagctcggtgaaaaccccgcttacgcagaaccagtacgatgcgctgtgcagcctggtatttaacatcggggctacagcattctacggttctacggtcctgaagcgagtgaaccagaaagactacaccgccgctgctgatgcgttcctgatgtggaagaaagccggcaaagaccaggaaattctactaccccggaggcggcgcgagcgtgcgctgttcctgtcgtga

SEQ ID NO: 40 - DNA, 495 bp, Klebsiella pneumoniae bacteriophage
atgaacccgacgctgaggaataagctcattggtgcgattgccggcggttcgggtgcgatcgcgattgcttctgtcatgcttggtaatgctgacggcctggaaggaaggcgttattacgcctatcaggatgttgtcggcgtctggactgtttgtgatggtcacactggcaccgatattcgccgcggccatcgttataccgacagggaatgcgacagcctgctgaaagccgatctgcggaaggtggcaagcgccattgatccgctcatcaaagtccgcattcctgatcctacccgcgccgcgctttactcattcacctacaacgttggctctggcgctttcgccagctccacgttgttgaagaaactgaatgctggagatgtgccgggcgcgtgcaaggaactgcagcgctggacgtatgccggtggcaagcagtggaaggggctgatcaccaggcgcgagattgagcgtgaagtctgcgagtggggccagaaatga

SEQ ID NO: 41 - DNA, 495 bp, Klebsiella pneumoniae bacteriophage
atgaacccgacgctgaggaataagttgattggtgcgatcgccggcggttctggtgcgatcgcaattgcttctgtcatgcttggaaatgcagacggcctggaaggaaggcgttattacgcctatcaggatgttgtcggcgtctggactgtttgtgatgggcacactggcaccgatattcgccgcggccaccgttacaccgaccgagaatgcgacaacctgctgaaggcagatctgcggaaggtggcaagcgccattgatccgctcatcaaagtccgccttcctgctcctacccgcgccgcgctttactcattcacttataacgttggctctggtgccttcgccagctccacgctactgaagaaactgaatgctggagacgtccctggcgcgtgcaaggaactgcagcgctggatgtatgccggtggcaagcagtggaagggcctgatcaccaggcgcgagattgagcgtgaagtctgcgagtggggccagaaatga

SEQ ID NO: 42 - DNA, 441 bp, Klebsiella pneumoniae bacteriophage
atgcaaaccagcgaaaagggcattgccctgatcaaagagttcgaaggctgcaaactcaccgcctaccaggacagcgtcggcgtctggacgatcggctatggctggactcatcctgtcgacggaaaaccaatccgcgccgggatgacgattaagcaggaaacggcagaacgcctgctgaaaactggactggtcagctacgaatgcgacgtgtctcgcctggttaaggtggggctgactcaagggcagttcgatgctctggtgtcgttcacgtataacctcggagcccgttcactgtcgacatcgactcttctgcgaaaactcaacgccggtgattacgctggcgcagccgatgagttcctgcgctggaataaagctggcggtaaagtcctgaatgggctcacccgtcgtcgggaggcagagcgggctctgttcctgtcatga

SEQ ID NO: 43 - DNA, 450 bp, Klebsiella pneumoniae bacteriophage
atgcaaatcagtgatgaaggcattgcgcttattaaaggtttcgaagggtgccgattgacagcatatcccgaccctggcaccggtggcgcaccatggaccataggttacggctggacattgccagttgatggcaagccggtacgtccgggtatgacgatcgatcaggctacagctgaccgcctgcttaaaatcggtctggtgggctacgaaaacgacgttctgaaaattgtgaaggtgaagctaacccaagggcagtttgatgccctggtgtcgtttgcctacaacatcggctcccgcgcactctcaacctccactctgctgaagaaacttaatgccggcgatatcaaagacgctgcagatgagttcctgcgttggaataaagcaggtggcaaggtcctgaatgggttgacccgtcggcgtgaggcggagcgcgctctgttcctgtcgtga

SEQ ID NO: 44 - DNA, 438 bp, Klebsiella pneumoniae bacteriophage
atgcaagtaagtgataacggtattgtttttttaaagaatgaagaaggcgaaaagttaacgggttacccggactcacgcggcattccaacaatcggcgtgggccacaccggaaaagttaacggtgtgccggtaagtgtcgggatgaaaataacatcagagcagtcgtcagaactgcttaaagatgatttaagctgggttgaagacagcattgcaaattatgttaaatcgccactgaatcagaatcagtatgacgcattgtgcagttttatcttcaatatcggcgcaccggcgtttgaaggttcaacaatgctcaagctgttaaacaagtcggattatgtcggcgcatccggtgaattcccgaaatggaagcgagccggtaatgacccggatattttgctgccgcgacgcatgcgcgaacaggctttatttttatcatga

SEQ ID NO: 45 - DNA, 438 bp, Klebsiella pneumoniae bacteriophage
atgcaaatcagcagtaacggaatcaccaaactcaaacgcgaagaaggcgagaggcttaaggcttacccagatagccgtggaatcccgacaatcggcgtgggccatacaggcaatgttgatggaaagcctgtaacacttggaatgacaatcacatcagataagtcatctgagcttctgaaagctgacttgcgatgggtggaagatgcaatcagcagcctggttcgcgttccactgactcaaaaccagtatgatgcgctttgcagtttgatattcaacattggtaaatctgcgtttgcaggctccactgttctgcgccaactaaaccttaagaattaccaggcagcggctgatgcattcctgatgtggaagaaagcaggtaaagatactgaaatcctacttccacggaggcagagagaaagggctctgttcctgtcatga

SEQ ID NO: 46 - DNA, 495 bp, Klebsiella pneumoniae bacteriophage
atgaacccgacgctcaggaataaactgattggcgccatcgccggaggttccggcgcgatcgcaattgcctctgtcatgcttggtaacgctgatgggctggaagggcggcgctattacgcttatcaggatgttgttggcgtctggactgtttgtgatggacataccggttcagatattcgccgcggtcaccgctactccgacaaagagtgcgataacctgctgaagtcagacctgcgaaaggttgctaacgccatcgacccgctgattaaggttcgcatccctgatcctacccgtgccgctctttactccttcacttataacgttggctctggtgccttcgccagttccacgctactgaagaaattgaatgctggagacgtgccgggtgcgtgcaaagaactgcagcgctggacgtatgccggtggcaagcaatggaagggcctaattacccgacgcgagattgagctcgaagtctgtgagtggggccagaaatga

SEQ ID NO: 47 - DNA, 498 bp, Klebsiella pneumoniae bacteriophage
atgaatcaacccttgcgaaaatatgtattgtctgcggtcggtggtggtgcaattgccatagcctctgcgcttatcactggccctacgggtaacgatggccttgagggtgtgcgatatcagccttaccaggatgtagttggcgtctggactgtctgctatggacacactggcaaagacattatgctggggaatacttacacgaaatcagagtgtgatgctcttctggataaagacctcaacaccgtcgctcgtcagattaacccgtacatcaaaaagccaatccctgaaacgatgcgtggggcgctgtactcatttgcctataacgttggtgctggcagctttcagacttcaacgctgctgcgcaaaattaaccagggggattcgaaaggtgcctgtgagcagttacgcgtctggatttacgcggggaaaaaggtctggaagggattggtaactcgccgtgaaattgagcgcgaggtgtgtttgtggggccaaaaatga

SEQ ID NO: 48 - DNA, 510 bp, Klebsiella pneumoniae bacteriophage
atgaatccttcaatcgttaagcgctgccttgtcggggcggtgctggctattgctgccacgctgcccggtttccagtcgcttcatacctccgttgaggggctgaaactgattgccgattacgaggggtgccgcctgcagccttatcagtgcagcgcgggcgtgtggaccgacgggatcggcaatacgtccggtgtggtgccgggcaaaaccatcacggaacggcaggcggcgcagggacttatcactaacgtactgcgcgtggagcgggcgctggataaatgtgtggcgcagccgatgccgcaaaaagtctatgacgcggtggtgtcgtttgctttcaacgtgggcaccggcaacgcctgcagctccacgctggttaagttgctgaaccagcggcgctgggcagatgcctgccatcagctgccgcgctgggtatatgtcaaaggtgtgtttaatcaggggctggacaatcgccgcgcgcgggaaatggcctggtgcttaaaaggagcataa

SEQ ID NO: 49 - DNA, 495 bp, Klebsiella pneumoniae bacteriophage
atggcaatgtcaccggcgctcagaaatagcattgttgcagccctcggtaccggtgctattggtatcgcgaccgtcatggtttctggaaagtcaggcctggagggtagagagcattacccatacaaagatattgttggcattgtcaccgtttgtgatgggtatacaggaagcgatattgtctggggtaaatattactcagacaaagaatgtgatgcgttgacgcgtaaagatatgacgcgaattgctgcacaagttaatccgcatatcaaagtgccgaccactgaaacacagcgagctgcaatatatagcttcgcttacaacgtcggatccacagcagccatcaactcaaccctgttgaagaaactcaactctaaagattactccggggcatgctcagagcttaagagatgggtatatgcaggtggaaagaaatggaaaggcctgatgaaccgacgcgacgttgagtacgaggtttgcacctggagccagaaatga

SEQ ID NO: 50 - DNA, 510 bp, Klebsiella pneumoniae bacteriophage
gtgagctcaatcgttaaacgttgcagtgtggccgcagtgctggcactggcggcattgttgcctgactttcgtctgctgcatacctcgcctgatggtctggcattgattgctgaccttgaagggtgccgcctggcaccttaccagtgcagtgcgggcgtgtggacgtcaggcatcggccacactgccggggtggtaccaaaacgcgatatcaccgagcgcgaagcggcggcaaatctggtcgccgacgtgctgaataccgagcgccgtctcgcggtctgcgtgccggtcaccatgccgcagcctgtttacgacgcgctggtcagtttctcttttaacgtcggcaccggcgcggcttgtcgctcgacgctggtctcttacatcaagcgtcatcagtggtggcaggcatgcgaccaacttagccgctgggtgtacgtcaacggggagcgtagcaccggacttgaaaatcgacgtcagcgagagcgtgcttattgcctgaagggggtgaaatga

SEQ ID NO: 51 - DNA, 528 bp, Klebsiella pneumoniae bacteriophage
atgacgaacaaagtaaagtttagcgctgccatgctggcgcttctcgctgccggagcaacagcaccagaattgtttgaccagttcatgagtgagaaagaaggtaatgcgctggtggctgtcgttgatcctggcggcgtctggtcgttatgtcatggcgttatctttatcgatggcaagcgtgtcgtaaaaggtatgacggcgactgagtctcaatgtcgaaaagtgaatgcaatcgagcgtgataaggcgctgtcgtgggttgaccgcaatatcaatgttcccctgaccgagccgcaaaaagtcggtattgcgtcattctgcccatacaacatcggcccaggtaaatgctttccttcgacgttttataagcgcatcaatgcaggtgaccgtaaaggggcatgcgaagcaatccgctggtggattaaggacggtgggaaggattgccgcatacgctctaataactgctacgggcaggtaactcgccgggatcaggaaagtgcgctgacgtgctgggggattgaccagtga

SEQ ID NO: 52 - DNA, 537 bp, Klebsiella pneumoniae bacteriophage
atgagcaacaaagctaaattcagcgccgctatgctggtgcttctggccgccggtgcgtcagcgccggtgctgttcgatcagtttattggtgaacgcgagggtaactcgctaacggcggttatcgatcccggtggggtttggtcaatatgccggggggtaacacgcatcgatggccgcccggtagtgaaggggatgaacttaacgcagagccagtgtgaccattacaacgcaatcgaacgcgacaaggcgctggcgtgggtacaaaagaatgttcacgttccactaactgagccgcagaaagtcggcattgccagcttttgcccgtacaacatcgggccggggaagtgttttccttcgacgttttatcgcaagctaaatgccggcgaccgcaaaggggcatgcgcggagatccggcgctggatattcgacggcggcagggattgccggttaacgaaagggcaggccaacggctgttacgggcaggttgaccgacgcgatcaggaaagtgcgctgacgtgctgggggctttacgaatga

SEQ ID NO: 53 - DNA, 534 bp, Klebsiella pneumoniae bacteriophage
atggcatccctgaaaacgaaactcagcgcagccatgctgggattaatagctgctggtgcatccgccccaaccttgatggatcagttcctggatgagaaagaaggtaacagccttaccgcttatcgcgatggtagccaggggatctggactatttgcagaggcgccacgcgaattgatggtaaacccgtcacgcagggaatgaagttgacccaggccaaatgcgacgaggtgaatgatatcgaacgtgataaggcactggcgtgggttgatcggaatatccgcgtaccgttgacgcctccgcagaaagtcggcattgcttcattctgtccgtacaacatcggccccggtaaatgcttcccgtctacgttctaccagcgcatcaacgccggcgaccgtaaaggcgcatgtgaagcgattcgctggtggattaaggacggtgggaaggattgccgcatacgctctaataactgctacgggcaggtaactcgccgggatcaggaaagtgcgctgacgtgctgggggattgaccagtga

SEQ ID NO: 54 - DNA, 495 bp, Klebsiella pneumoniae bacteriophage
atggcatggggtgccaaggttagtaaagagttcaagttaaaggtgattgaggtgtgcgaacgccttgaaattaaccctgactacttgatgagctgcatggcttttgaaacgggcgagacgttctcaccaaatgtccgcaatccgaatgggtccgccactggcttgatccagtttatgtccaacacagctcgcagtctgggtactacgacaaatgagttagcagacatgacctctgttgagcaaatggactacgtggagaagtactttaagccgtatgctgggaaaatcaagacgattgaggatgtatacatggtgattttttgccctcgtgccgttggaaaacctgactcgtatattctttacgacgaaggtcgtagttacaacgacaataaagggttggaccttaataaggacaatgctattactaaatacgaggctggattcaaggtgcgtgagaaactgaagttaggtatgaaagagggttaccgtggttaa

SEQ ID NO: 55 - DNA, 528 bp, Klebsiella pneumoniae bacteriophage
atggctaacctgaaaacgaaactcagttcggccatgctggcgcttatcgctgctggcgcttcagctcccgttcttatggaccagttcctgaatgagaaagagggcaaaagcctcacgtcataccgcgatggcgccggcatatggacgatatgtcgtggagttacccaggtagatggaagacctgtaacccagggaatgaagttaacccaggccaaatgcgatcaggttaatgccgtcgagcgcaataaggcgctggcatgggtagatcagaatgtgcgtgttcctctgacaccccctcaaaaggtcgggattgccagtttctgcccctataacatcgggcccggtaaatgctttccttccaccttctaccgcaagctgaatgccggtgaccggaaaggcgcctgcgctgaaattcgccggtggatttttgatggcggaaaagattgccgcgtgcgttcgaacaattgttacggccaggtctctcgtcgtgatcaggaaagcgcactggcatgttgggggatagatgaa

SEQ ID NO: 56 - DNA, 600 bp, Klebsiella pneumoniae bacteriophage
atgcagataagcgataacggcatcgcactgattaaggggtttgaaggatgtcgattaaccgcatacccggacccgggcaccggcggtgatccctggacgattggtttcggctggacggggaaagtagacggcaagcctatcaagccgggaatgaagattgacgatgcgacagcggatcgcctgctgcgcactggcgtggtgagctttgacctggcggtaagcaagatgctcaaagtttccgtcacccagaatcagtacgacgcgcttgtgtcgctggcctataacatcggtacgcgagcgctatccacctcaacgctgatgaaaaagctgaatgcaggtgatgtgaaaggcgcagctgatgagttccttcgctggaataaatcaggcgggaaagcaatgtctgggctaaccaatcgccgcaaggcagagcgagaagtatttttatcgaaaacacggggaagttatgaactatctcatggtaccggaggtggatcaggtggaggttctggaggaggtgaccatgagtgtcactatcgtatcaaaccgacatttcgccgtctgaaatggaagtataaaggtaaattttggtgccccagttaa

SEQ ID NO: 57 - DNA, 627 bp, Klebsiella pneumoniae bacteriophage
atgcagataagcgataacggcatcgcactgattaaggggtttgaaggatgtcgattaaccgcatacccggacccgggcaccggcggtgatccctggacgattggtttcggctggacggggaaagtagacggcaagcctatcaagccgggaatgaagattgacgatgcgacagcggatcgcctgctgcgcactggcgtggtgagctttgacctggcggtaagcaagatgctcaaagtttccgtcacccagaatcagtacgacgcgcttgtgtcgctggcctataacatcggtacgcgagcgctatccacctcaacgctgatgaaaaagctgaatgcaggtgatgtgaaaggcgcagctgatgagttccttcgctggaataaatcaggcgggaaagcaatgtctgggctaaccaatcgccgcaaggcagagcgagaagtatttttatcgaaaacacggggaagttatgaactatctcatggtaccggaggtggatcaggtggaggttctggaggaggtttgcttggagacttttttcgcaaatccaaggagaaaattggcaaggaattcaagcgtattgtacagcgcatcaaggactttctgcgcaacttggtcccgcgtacagaaagt

SEQ ID NO: 58 - DNA, 570 bp, Klebsiella pneumoniae bacteriophage
atgcagataagcgataacggcatcgcactgattaaggggtttgaaggatgtcgattaaccgcatacccggacccgggcaccggcggtgatccctggacgattggtttcggctggacggggaaagtagacggcaagcctatcaagccgggaatgaagattgacgatgcgacagcggatcgcctgctgcgcactggcgtggtgagctttgacctggcggtaagcaagatgctcaaagtttccgtcacccagaatcagtacgacgcgcttgtgtcgctggcctataacatcggtacgcgagcgctatccacctcaacgctgatgaaaaagctgaatgcaggtgatgtgaaaggcgcagctgatgagttccttcgctggaataaatcaggcgggaaagcaatgtctgggctaaccaatcgccgcaaggcagagcgagaagtatttttatcgaaaacacggggaagttatgaactatctcatggtaccggaggtggatcaggtggaggttctggaggaggtcgcaagaagactcgtaagcgcctgaagaaaatcgggaaggtgttaaaatggatt

SEQ ID NO: 59 - DNA, 33 bp, Artificial Sequence
  primer
cccgtcgacatggctaacctgaaaacgaaactc

SEQ ID NO: 60 - DNA, 34 bp, Artificial Sequence
  primer
cccgcggccgctcattcatctatcccccaacatg

SEQ ID NO: 61 - DNA, 40 bp, Artificial Sequence
  primer
aattcgtcgacggggcggccgcggtacctctagactgcag

SEQ ID NO: 62 - DNA, 31 bp, Artificial Sequence
  primer
gtctagaggtaccgcggccgccccgtcgacg

SEQ ID NO: 63 - protein, 174 aa, Pseudomonas aeruginosa bacteriophage
MKLAWGKKVDQAFRDKVFAICDGFKWNRETHASWLMSCMAFESGETFSPSVRNAAGSGATGLIQFMPRTAQGLGTSTAELAAMSAVDQLDYVQKYFRPYASRIGTLSDMYMAILMPKFVGQPEDSVLFLDPKISYRQNAGLDANRDGKITKAEAASKVRAKFDKGMLDRFALEL

SEQ ID NO: 64 - protein, 174 aa, Pseudomonas aeruginosa bacteriophage
MAWSAKVSQAFCDRVIWIAASLGMPADGADWLMACIAWETGETFSPSVRNGAGSGATGLIQFMPATARGLGTTTDELARMTPEQQLDYVYRYFLPYRGRLKSLADTYMAILWPAGIGRALDWALWDSTSRPTTYRQNAGLDINRDGVITKAEAAAKVQAKLDRGLQPQFRRAAA

SEQ ID NO: 65 - protein, 220 aa, Pseudomonas aeruginosa bacteriophage
MKITKDVLITGTGCTTDRAIKWLDDVQAAMDKFHIESPRAIAAYLANIGVESGGLVSLVENLNYSAQGLANTWPRRYAVDPRVRPYVPNALANRLARNPVAIANNVYADRMGNGCEQDGDGWKYRGRGLIQLTGKSNYSLFAEDSGMDVLEKPELLETPAGASMSSAWFFWRNRCIPMAESNNFSMVVKTINGAAPNDANHGQLRINRYLKTIAAINQGS

SEQ ID NO: 66 - protein, 154 aa, Pseudomonas aeruginosa bacteriophage
MKGKVIGGSAAAVIALAAAALVKPWEGYSPTPYIDMVGVATHCYGDTSRADKAVYTEQECAEKLNSRLGSYLTGISQCIKVPLREREWAAVLSWTYNVGVGAACRSTLVGRINAGQPAASWCPELDRWVYAGGKRVQGLVNRRAAERRMCEGRS

SEQ ID NO: 67 - protein, 144 aa, Pseudomonas aeruginosa bacteriophage
MRTSQRGIDLIKGFEGLRLSAYQDSVGVWTIGYGTTRGVTRYMTITVEQAERMLSNDLRRFEPELDRLVKAPLNQNQWDALMSFVYNLGAANLASSTLLKLLNKGDYQGAADQFPRWVNAGGKRLEGLVKRRAAERVLFLEPLS